(19) United States
(12) Patent Application Publication
(10) Pub. No.: US 2024/0287512 A1
(43) Pub. Date: Aug. 29, 2024

(54) LIPID CONJUGATION FOR TARGETING NEURONS OF THE CENTRAL NERVOUS SYSTEM

(71) Applicant: Dicerna Pharmaceuticals, Inc., Lexington, MA (US)

(72) Inventors: Travis Grim, Maynard, MA (US); Matthew Costales, Lexington, MA (US); Bob Dale Brown, Littleton, MA (US); Maire Jung, Lincoln, MA (US)

(21) Appl. No.: 18/290,211

(22) PCT Filed: May 11, 2022
(86) PCT No.: PCT/US22/28718

§ 371 Date: Nov. 10, 2023

Related U.S. Application Data
(60) Provisional application No. 63/276,404, filed on Nov. 5, 2021.
(60) Provisional application No. 63/187,250, filed on May 11, 2021.

(51) Int. Cl. C12N 15/113

(52) U.S. Cl. CPC C12N 15/113; C12N 2310/11; C12N 2310/315; C12N 2310/321; C12N 2310/322; C12N 2310/3515

(57) ABSTRACT

Oligonucleotide conjugates are provided herein that inhibit or reduce expression of target genes in the neurons of the central nervous system. Also provided are compositions including the same and uses thereof, particularly uses relating to treating diseases, disorders and/or conditions associated with expression of a neuronal target gene in the CNS.

### CROSS-RELATED APPLICATIONS

This application claims the benefit of U.S. Provisional Patent Application Ser. No. 63/187,250 filed May 11, 2021, and U.S. Provisional Patent Application Ser. No. 63/276,404 filed Nov. 5, 2021. The entire contents of which are incorporated herein by this reference.

### REFERENCE TO SEQUENCE LISTING

The instant application contains a Sequence Listing which has been submitted electronically in ASCII format via EFS-Web, and is hereby incorporated by reference in its entirety. Said ASCII copy, created on May 9, 2022, is named DICN_007_001WO_SeqList_ST25 and is 47,059 bytes in size.

### TECHNICAL FIELD

The disclosure relates to oligonucleotides linked to lipid moieties useful in the inhibition of target genes in neurons of the central nervous system. Specifically, the present disclosure relates to oligonucleotide-lipid conjugates, methods to prepare them, their chemical configuration, and methods to modulate (e.g., inhibit or reduce) the expression of a target gene in a neuron of the central nervous system (abbreviated “CNS” hereinafter) (e.g., tissue, or region of the CNS) using the conjugated nucleic acids and oligonucleotides according to the description provided herein. The disclosure also provides pharmaceutically acceptable compositions comprising the conjugates of the present description and methods of using said compositions in the treatment of various diseases or disorders.

### BACKGROUND OF THE DISCLOSURE

Regulation of gene expression by modified nucleic acids shows great potential as both a research tool in the laboratory and a therapeutic approach in the clinic. Several classes of oligonucleotide or nucleic acid-based therapeutics have been under the clinical investigation, including antisense oligonucleotides (ASO), short interfering RNA (siRNA), double-stranded nucleic acids (dsNA), aptamers, ribozymes, exon-skipping and splice-altering oligonucleotides, immunomodulatory oligonucleotides, mRNAs, and CRISPR. Chemical modifications in the relevant molecules to allow functionality in various tissues, organs and/or cell types play a key role in overcoming challenges of oligonucleotide therapeutics, including improving nuclease stability, RNA-binding affinity, and pharmacokinetics. Various chemical modification strategies for oligonucleotides have been developed in the past three decades including modification of the sugars, nucleobases, and phosphodiester backbone to improve and optimize performance and therapeutic efficacy (Deleavey and Darma, CHEM. BIOL. 2012, 19(8):937-54; Wan and Seth, J. MED. CHEM. 2016, 59(21):9645-67; and Egli and Manoharan, Acc. CHEM. RES. 2019, 54(4):1036-47).

Therapeutic gene silencing mediated by RNAi oligonucleotide-based therapeutics comprising siRNAs or double-stranded nucleic acids (dsNAs) offer the potential for considerable expansion of the druggable target space and the possibility for treating orphan diseases that may be therapeutically unapproachable by other drug modalities (e.g., antibodies and/or small molecules). RNAi oligonucleotide-based therapeutics that inhibit or reduce expression of specific target genes in the liver have been developed and are currently in clinical use (Sehgal et al., (2013) JOURNAL OF HEPATOLOGY 59:1354-59). Technological hurdles remain for the development and clinical use of RNAi oligonucleotides in extrahepatic cells, tissues, and organs (e.g., the central nervous system or CNS). Therapeutic gene silencing mediated by RNAi oligonucleotide-based therapeutics in the CNS is of particular interest to treat neurological diseases (Boudreau & Davidson (2010) BRAIN RESEARCH 1338:112-21). Thus, an ongoing need exists in the art for the successful development of new and effective RNAi oligonucleotides to modulate the expression of a target genes in extrahepatic cells, tissues, and/or organs (e.g., the CNS). This is complicated by the variant nature of the cell types in extrahepatic as well as concerns about circulatory patterns and cell membrane constituents such as receptor types.

### BRIEF SUMMARY OF THE DISCLOSURE

The mammalian CNS is a complex system of tissues, including cells, fluids and chemicals that interact in concert to enable a wide variety of functions, including movement, navigation, cognition, speech, vision, and emotion. Unfortunately, a variety of diseases and disorders of the CNS are known (e.g., neurological disorders) and affect or disrupt some or all of these functions. Typically, treatments for diseases and disorders of the CNS have been limited to small molecule drugs, antibodies and/or to adaptive or behavioral therapies. There exists an ongoing need to develop treatment of diseases and disorders of the CNS associated with inappropriate gene expression.

The present disclosure is based, at least in part, on the discovery of lipid-conjugated RNAi oligonucleotides that effectively reduce target gene expression in neurons of the CNS. Exemplary lipid-conjugated RNAi oligonucleotides provided herein have demonstrated reduction of target gene expression of neuron-specific mRNA in the CNS following a single administration. Further, exemplary lipid-conjugated RNAi oligonucleotides provided herein have demonstrated pharmacological activity in multiple regions throughout the CNS, including difficult to reach areas such as the hippocampus and frontal cortex. Without being bound by theory, the hydrophobic moiety (e.g., lipid) facilitates delivery and distribution of the lipid-conjugated RNAi oligonucleotides into the CNS, thereby increasing efficacy and durability of gene knockdown in neurons. Accordingly, the disclosure provides methods of treating a disease or disorder by modulating expression of a neuronal gene in the CNS using the lipid-conjugated RNAi oligonucleotides, and pharmaceutically acceptable compositions thereof, described herein. The disclosure further provides methods of using the lipid-conjugated RNAi oligonucleotides in the manufacture of a medicament for treating a disease or disorder by modulating expression of a neuronal gene in the CNS.

Accordingly, in some aspects, the disclosure provides a double-stranded oligonucleotide comprising an antisense strand of 15-30 nucleotides in length and a sense strand of 15-50 nucleotides in length, wherein the antisense and sense strands form a duplex region of 15-30 base pairs, wherein the antisense strand comprises a region of complementarity to a neuronal mRNA target sequence, and wherein the sense strand comprises at least one lipid moiety conjugated to the 5′ terminal nucleotide of the sense strand.

The disclosure is further based in part on the discovery of lipid-conjugated RNAi oligonucleotides having a stem-loop that effectively reduce target gene expression in neurons of certain tissues of the CNS. Specifically, exemplary lipid-conjugated RNAi oligonucleotides having a stem-loop demonstrated reduction of target gene expression of neuron-specific mRNA in the spinal cord following a single administration, without reducing expression of the target gene to the same level in other tissues of the CNS (e.g., medulla, cerebellum, hippocampus, frontal cortex). Without being bound by theory, the ability of lipid-conjugated RNAi oligonucleotides having a stem-loop to preferentially reduce expression of a neuronal mRNA in the spinal cord indicates such oligonucleotides are useful for treating diseases of the spinal cord without impacting other regions of the CNS. Accordingly, the disclosure provides methods of treating a disease or disorder my modulating expression of a neuronal gene in the spinal cord using the lipid-conjugated RNAi oligonucleotides, and pharmaceutically acceptable compositions thereof, described herein. The disclosure further provides methods of using the lipid-conjugated RNAi oligonucleotides in the manufacture of a medicament for treating a disease or disorder by modulating expression of a neuronal gene in the spinal cord.

Accordingly, in some aspects, the disclosure provides a double-stranded oligonucleotide comprising an antisense strand of 15-30 nucleotides in length and a sense strand of 15-50 nucleotides in length, wherein the antisense and sense strands form a duplex region of 15-30 base pairs, wherein the antisense strand comprises a region of complementarity to a neuronal mRNA target sequence, and wherein the sense strand comprises (i) at least one lipid moiety conjugated to a nucleotide of the sense strand, and (ii) a stem-loop, wherein the stem-loop comprises a nucleotide sequence represented by the formula: 5′-S1-L-S2-3′, wherein S1 is complementary to S2, and wherein L forms a loop between S1 and S2.

In any of the foregoing or related aspects, the lipid moiety is selected from

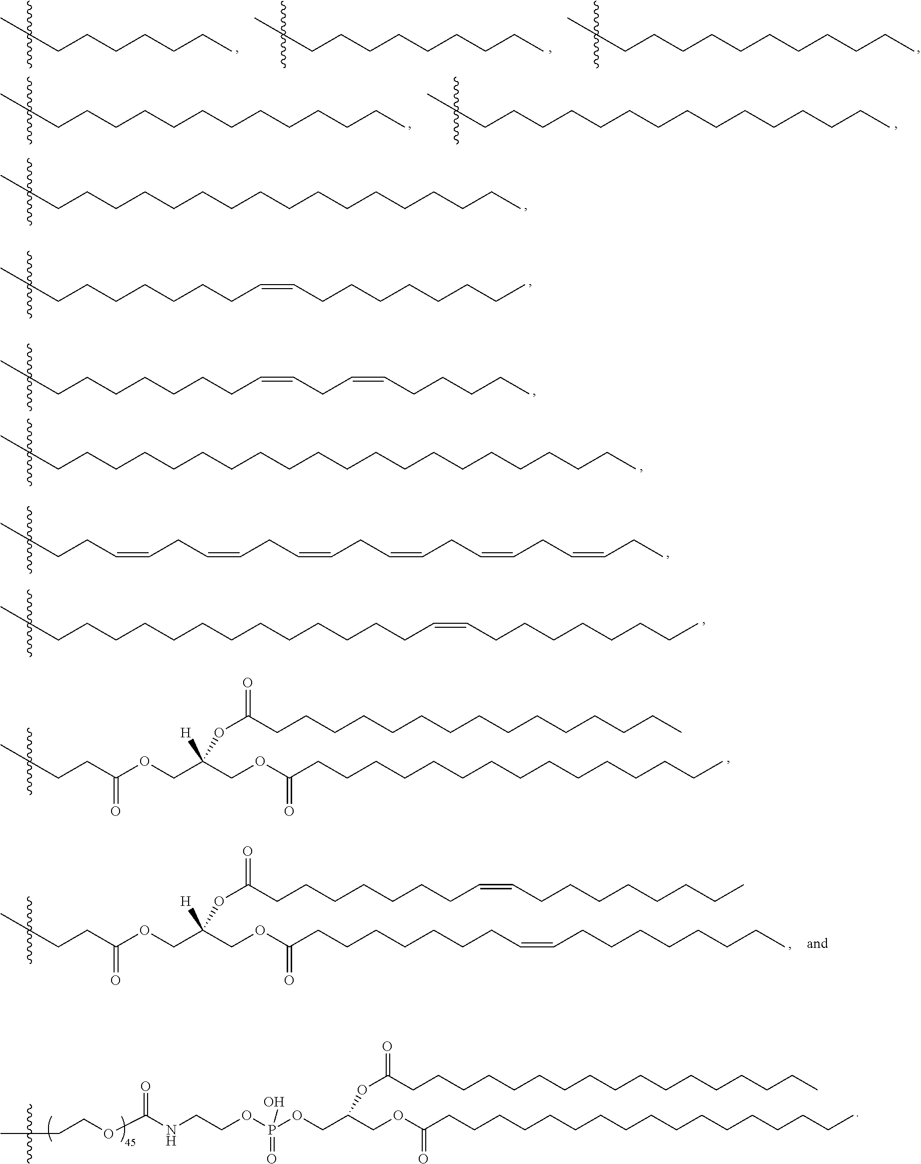

In some aspects, the lipid moiety is a hydrocarbon chain. In some aspects, the hydrocarbon chain is a C₈-C₃₀ hydrocarbon chain. In some aspects, the hydrocarbon chain is a C16 hydrocarbon chain. In some aspects, the C16 hydrocarbon chain is represented by

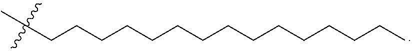 [SMILES: *CCCCCCCCCCCCCCC]

In any of the foregoing or related aspects, the lipid moiety is conjugated to the 2′ carbon of the ribose ring of the 5′ terminal nucleotide.

In any of the foregoing or related aspects, the oligonucleotide is blunt ended. In some aspects, the oligonucleotide is blunt ended at the 3′ terminus of the oligonucleotide.

In some aspects, the oligonucleotide comprises a blunt end. In some aspects, the blunt end comprises the 3′ terminus of the sense strand.

In any of the foregoing or related aspects, the antisense strand comprises a 1-4 nucleotide overhang at the 3′ terminus. In some aspects, the overhang comprises purine nucleotides. In some aspects, the overhang sequence is 2 nucleotides in length. In some aspects, the overhang is selected from AA, GG, AG, and GA. In some aspects, the overhang is GG or AA. In some aspects, the overhang is GG.

In any of the foregoing or related aspects, the sense strand is 20-22 nucleotides and the antisense strand is 22-24 nucleotides. In some aspects, the duplex region is 20-22 base pairs. In some aspects, the sense strand is 20 nucleotides and the antisense strand is 22 nucleotides, and wherein the duplex region is 20 base pairs.

In any of the foregoing or related aspects, the sense strand is 36-38 nucleotides and the antisense strand is 22-24 nucleotides. In some aspects, the sense strand is 36 nucleotides and the antisense strand is 22 nucleotides, and wherein the duplex region is 20 base pairs.

In any of the foregoing or related aspects, the sense strand is 36 nucleotides and comprises positions 1-36 from 5′ to 3′, and wherein the lipid moiety is conjugated at position 1, position 7, position 9, position 10, position 16, position 20, position 23, position 28, position 29 or position 30. In some aspects, the lipid moiety is conjugated at position 28. In some aspects, the antisense strand is 22 nucleotides, and the duplex region is 20 base pairs.

In other aspects, the disclosure provides a double-stranded oligonucleotide comprising an antisense strand of 22-24 nucleotides in length and a sense strand of 20-22 nucleotides in length, wherein the antisense and sense strands form an asymmetric duplex region of 20-22 base pairs having a 2 nucleotide overhang on the 5′ end of the oligonucleotide and a blunt-end on the 3′ end of the oligonucleotide, wherein the antisense strand comprises a region of complementarity to a neuronal mRNA target sequence, and wherein the sense strand comprises at least one lipid moiety conjugated to the 5′ terminal position on the sense strand. In some aspects, the disclosure provides a double-stranded oligonucleotide comprising an antisense strand of 22-24 nucleotides in length and a sense strand of 20-22 nucleotides in length, wherein the antisense and sense strands form an asymmetric duplex region of 20-22 base pairs having a 2 nucleotide overhang on the 3′ end of the antisense strand and a blunt-end comprising the 3′ end of the sense strand and 5′ end of the antisense strand, wherein the antisense strand comprises a region of complementarity to a neuronal mRNA target sequence, and wherein the sense strand comprises at least one lipid moiety conjugated to the 5′ terminal position on the sense strand.

In some aspects, the lipid moiety is a C16 hydrocarbon chain. In some aspects, the C16 hydrocarbon chain is represented by

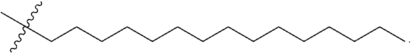 [SMILES: *CCCCCCCCCCCCCCC]

In some aspects, the antisense strand is 22 nucleotides and the sense strand is 20 nucleotides. In some aspects, the 2-nucleotide overhang comprises purines. In some aspects, the overhang is selected from AA, GG, AG, and GA.

In any of the foregoing or related aspects, the region of complementarity is complementary to at least 15 consecutive nucleotides of the neuronal mRNA target sequence. In some aspects, the region of complementarity is complementary to at least 19 consecutive nucleotides of the neuronal mRNA target sequence.

In any of the foregoing or related aspects, the oligonucleotide comprises at least one modified nucleotide. In some aspects, the modified nucleotide comprises a 2′-modification. In some aspects, all of the nucleotides of the sense strand and the antisense strand comprise a 2′-modification except the 5′-terminal nucleotide of the sense strand. In some aspects, all of the nucleotides of the sense strand and the antisense strand comprise a 2′-modification except the nucleotide conjugated to the lipid moiety. In some aspects, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some aspects, about 10-20%, 10%, 11%, 12%, 13%, 14% 15%, 16%, 17%, 18%, 19% or 20% of the nucleotides of the sense strand comprise a 2′-fluoro modification. In some aspects, about 25-35%, 25%, 26%, 27%, 28%, 29%, 30%, 31%, 32%, 33%, 34% or 35% of the nucleotides of the antisense strand comprise a 2′-fluoro modification. In some aspects, about 25-35%, 25%, 26%, 27%, 28%, 29%, 30%, 31%, 32%, 33%, 34% or 35% of the nucleotides of the oligonucleotide comprise a 2′-fluoro modification. In some aspects, the sense strand comprises 20 nucleotides with positions 1-20 from 5′ to 3′, wherein each of positions 8-11 comprise a 2′-fluoro modification. In some aspects, the sense strand comprises 36 nucleotides with positions 1-36 from 5′ to 3′, wherein each of positions 8-11 comprise a 2′-fluoro modification. In some aspects, the sense strand comprises 36 nucleotides with positions 1-36 from 5′ to 3′, wherein each of positions 8, 10 and 11 comprise a 2′-fluoro modification. In some aspects, the sense strand comprises 36 nucleotides with positions 1-36 from 5′ to 3′, wherein each of positions 8, 9 and 11 comprise a 2′-fluoro modification. In some aspects, the antisense strand comprises 22 nucleotides with positions 1-22 from 5′ to 3′, and wherein each of positions 2, 3, 4, 5, 7, 10 and 14 comprise a 2′-fluoro modification. In some aspects, the remaining nucleotides comprise a 2′-O-methyl modification except the 5′-terminal nucleotide of the sense strand. In some aspects, the remaining nucleotides comprise a 2′-O-methyl modification except the nucleotide conjugated to the lipid moiety.

In any of the foregoing or related aspects, the oligonucleotide comprises at least one modified internucleotide linkage. In some aspects, the at least one modified internucleotide linkage is a phosphorothioate linkage. In some aspects, the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′. In some aspects, the antisense strand is 22 nucleotides in length, and wherein the antisense strand comprises a phosphorothioate linkage between positions 20 and 21 and between positions 21 and 22, wherein positions are numbered 1-22 from 5′ to 3′. In some aspects, the sense strand comprises a phosphorothioate linkage between position 1 and 2, wherein positions are numbered 1-2 from 5′ to 3′. In some aspects, the sense strand is 20 nucleotides in length, and wherein the sense strand comprises a phosphorothioate linkage between positions 18 and 19, and between positions 19 and 20, wherein positions are numbered 1-22 from 5′ to 3′.

In any of the foregoing or related aspects, the antisense strand comprises a phosphorylated nucleotide at the 5′ terminus, wherein the phosphorylated nucleotide is selected from uridine and adenosine. In some aspects, the phosphorylated nucleotide is uridine. In some aspects, the 4′-carbon of the sugar of the 5′-nucleotide of the antisense strand comprises a phosphate analog. In some aspects, the phosphate analog is oxymethyl phosphonate, vinyl phosphonate or malonyl phosphonate.

In any of the foregoing or related aspects, the region of complementary is fully complementary to the neuronal mRNA target sequence at nucleotide positions 2-8 of the antisense strand, wherein nucleotide positions are numbered 5′ to 3′. In some aspects, the region of complementary is fully complementary to the neuronal mRNA target sequence at nucleotide positions 2-11 of the antisense strand, wherein nucleotide positions are numbered 5′ to 3′.

In any of the foregoing or related aspects, a lipid moiety is conjugated to the 2′ carbon of the ribose ring of a nucleotide of the sense strand. In some aspects, a lipid moiety is conjugated to the 2′ carbon of the ribose ring of a nucleotide of a loop.

In any of the foregoing or related aspects, the oligonucleotide is a Dicer substrate. In some aspects, the oligonucleotide is a Dicer substrate that, upon endogenous Dicer processing, yields double-stranded nucleic acids of 19-21 nucleotides in length capable of reducing a neuronal mRNA expression in a mammalian cell.

In any of the foregoing or related aspects, the neuronal mRNA target sequence is located in a region of the central nervous system (CNS). In some aspects, the region of the CNS is selected from the lumbar spinal cord, lumbar dorsal root ganglion, medulla, hippocampus, somatosensory cortex, frontal cortex, and a combination thereof. In some aspects, the region of the CNS is selected from the spinal cord, lumbar spinal cord, lumbar dorsal root ganglion, cervical spinal cord, thoracic spinal cord, medulla, hippocampus, somatosensory cortex, frontal cortex, and a combination thereof. In some embodiments, the region of the CNS is the spinal cord. In some aspects, the region of the CNS is the spinal cord. In some aspects, the spinal cord comprises the lumbar spinal cord, the thoracic spinal cord, and the cervical spinal cord. In some aspects, the region of the CNS is the lumbar spinal cord. In some aspects, the region of the CNS is the lumbar dorsal root ganglion. In some aspects, the region of the CNS is the thoracic spinal cord. In some aspects, the region of the CNS is the cervical spinal cord. In some aspects, the region of the CNS is the medulla. In some aspects, the region of the CNS is the hippocampus. In some aspects, the region of the CNS is the somatosensory cortex. In some aspects, the region of the CNS is the frontal cortex.

In any of the foregoing or related aspects, the oligonucleotide reduces expression of a target mRNA in a neuron or population of neurons in vitro and/or in vivo.

In any of the foregoing or related aspects, the oligonucleotide reduces expression of a target mRNA in a neuron or population of neurons in the spinal cord. In some aspects, the oligonucleotide reduces expression of a target mRNA in a neuron or population of neurons in the spinal cord relative to expression of the target mRNA in other regions of the CNS.

In some aspects, the disclosure provides a pharmaceutical composition an oligonucleotide described herein, and a pharmaceutically acceptable carrier, delivery agent or excipient.

In other aspects, the disclosure provides a method for treating a subject having a disease, disorder or condition associated with expression of a neuronal mRNA, the method comprising administering to the subject a therapeutically effective amount of an oligonucleotide or pharmaceutical composition described herein, thereby treating the subject. In some aspects, the disease, disorder or condition is acute or chronic pain. In some aspects, the disease, disorder or condition is a neurodegenerative disease.

In yet other aspects, the disclosure provides a method of delivering an oligonucleotide to a neuron or a population of neurons in a subject, the method comprising administering a pharmaceutical composition described herein to the subject. In some aspects, the neuron or population of neurons is located in a region of the CNS. In some aspects, region of the CNS is selected from the lumbar spinal cord, lumbar dorsal root ganglion, medulla, hippocampus, somatosensory cortex, frontal cortex, and a combination thereof. In some aspects, region of the CNS is selected from the spinal cord, lumbar spinal cord, thoracic spinal cord, cervical spinal cord, lumbar dorsal root ganglion, medulla, hippocampus, somatosensory cortex, frontal cortex, and a combination thereof. In some aspects, the region of the CNS is the spinal cord. In some aspects, the spinal cord comprises the lumbar spinal cord, the thoracic spinal cord, and the cervical spinal cord.

In further aspects, the disclosure provides a method for reducing expression of a neuronal mRNA in a cell, a population of cells or a subject, the method comprising the step of:
i. contacting the cell or the population of cells with an oligonucleotide or pharmaceutical composition described herein, optionally wherein the cell or population of cells is a neuron or population of neurons; or
ii. administering to the subject an oligonucleotide or pharmaceutical composition described herein. In some aspects, reducing expression of the neuronal mRNA comprises reducing an amount or level of mRNA, an amount or level of protein, or both. In some aspects, the subject has a disease, disorder or condition associated with expression of the neuronal mRNA. In some aspects, the disease, disorder or condition is acute or chronic pain. In some aspects, the disease, disorder, or condition is a neurodegenerative disease. In some aspects, the cell or population of cells is located in a region of the CNS. In some aspects, the region of the CNS is selected from the lumbar spinal cord, lumbar dorsal root ganglion, medulla, hippocampus, somatosensory cortex, frontal cortex, and a combination thereof. In some aspects, administering is intrathecal. In some aspects, the region of the CNS is selected from the spinal cord, lumbar spinal cord, lumbar dorsal root ganglion, thoracic spinal cord, cervical spinal cord, medulla, hippocampus, somatosensory cortex, frontal cortex, and a combination thereof. In some aspects, the region of the CNS is the spinal cord. In some aspects, the spinal cord comprises the lumbar spinal cord, the thoracic spinal cord, and the cervical spinal cord.

In other aspects, the disclosure provides a kit comprising an oligonucleotide described herein, an optional pharmaceutically acceptable carrier, and a package insert comprising instructions for administration to a subject having a disease, disorder or condition associated with expression of a neuronal mRNA. In some aspects, the package insert comprises instructions for intrathecal administration.

In further aspects, the disclosure provides use of an oligonucleotide or pharmaceutical composition described herein, in the manufacture of a medicament for the treatment of a disease, disorder or condition associated with expression of a neuronal mRNA. In some aspects, the treatment of a disease, disorder or condition associated with expression of a neuronal mRNA. In some aspects, the disease, disorder or condition is acute or chronic pain. In some aspects, the disease, disorder, or condition is a neurodegenerative disease.

### BRIEF DESCRIPTION OF FIGURES

FIG. 1 provides a schematic of a lipid-conjugated RNAi oligonucleotides and positions for conjugation of the lipid onto the sense strand. Arrows indicate nucleotide location on the sense strand for conjugation of a C16 lipid. The conjugation is on position 28 (P28) of the sense strand of (i) (referred to herein as “reference oligonucleotide”), and any one of positions 1 (P1), 7 (P7), 9 (P9), 10 (P10), 16 (P16), or 20 (P20) of the sense strand of (ii).

FIGS. 2A-2F provide graphs depicting the percent (%) murine Tubb3 mRNA remaining in lumbar spinal cord (FIG. 2A), lumbar dorsal root ganglion (FIG. 2B), medulla (FIG. 2C), hippocampus (FIG. 2D), somatosensory cortex (FIG. 2E), and frontal cortex (FIG. 2F) of mice after treatment with lipid-conjugated Tubb3 blunt-end oligonucleotides or the reference oligonucleotide. Mice were dosed intrathecal (i.t.) into cerebrospinal fluid (CSF) with 500 μg of the indicated Tubb3 lipid-conjugated oligonucleotides in Table 1 formulated in artificial cerebrospinal fluid (aSCF). Seven days post dose, the level of Tubb3 mRNA was normalized to Ribosomal Protein L23 (RPL23) mRNA and overall expression was determined between tissue types relative to control mice treated with (aSCF).

FIGS. 3A-3B provide graphs depicting the percent (%) murine Tubb3 mRNA remaining in different tissues of the central nervous system (CNS) based on the results in FIGS. 2A-2F. Tissue furthest away from the site of injection is shown from left to right.

FIGS. 4A-4B provide graphs depicting tissue concentration of lipid-conjugated Tubb3 RNAi oligonucleotides in different tissues of the central nervous system (CNS) from mice treated in FIGS. 2A-2F.

FIGS. 5A-5F provide graphs depicting the relationship between the percent (%) remaining Tubb3 mRNA in as shown in FIGS. 2A-2F to the concentration (ng/g) of lipid-conjugated Tubb3 RNAi oligonucleotide remaining in the tissue as shown in FIGS. 4A-4B for the lumbar dorsal root ganglion (FIG. 5A), the lumbar spinal cord (FIG. 5B), hippocampus (FIG. 5C), and medulla (FIG. 5D), frontal cortex (FIG. 5E), and somatosensory cortex (FIG. 5F).

FIG. 6 provides a schematic of a lipid-conjugated RNAi oligonucleotide and exemplary positions for conjugation of lipids onto the sense strand of oligonucleotides having a tetraloop. Arrows indicate the nucleotide positions on the sense strand conjugated to a C16 lipid. The conjugation is on position 1 (P1), 7 (P7), 9 (P9), 10 (P10), 16 (P16), 20 (P20), 23 (P23), 28 (P28), 29 (P29), or 30 (P30) of the sense strand (as indicated by the arrows).

FIGS. 7A-7F provide graphs depicting the percent (%) murine Tubb3 mRNA remaining in lumbar spinal cord (FIG. 7A), lumbar dorsal root ganglion (FIG. 7B), medulla (FIG. 7C), cerebellum (FIG. 7D), hippocampus (FIG. 7E), and frontal cortex (FIG. 7F) of mice after treatment with lipid-conjugated Tubb3 tetraloop oligonucleotides. Mice were treated with 500 μg of the indicated Tubb3 lipid-conjugated tetraloop oligonucleotides in Table 2 formulated in artificial cerebrospinal fluid (aSCF) via intrathecal injection into the lumbar spine. Seven (7) days following intrathecal injection, the level of Tubb3 mRNA was normalized to Ribosomal Protein L23 (RPL23) mRNA and overall expression was determined between tissue types relative to control mice treated with (aCSF).

FIGS. 8A-8D provide graphs comparing the percent (%) remaining Tubb3 mRNA as shown in FIGS. 2A, 2C, 2D, and 2F evaluating blunt-end oligonucleotides to the percent (%) remaining Tubb3 mRNA as shown in FIGS. 7A, 7C, 7E, and 7F evaluating tetraloop oligonucleotides for the lumbar spinal cord (FIG. 8A), medulla (FIG. 8B), hippocampus (FIG. 8C), and frontal cortex (FIG. 8D).

FIGS. 9A-9B provide graphs depicting concentration-response relationships relating the percent (%) murine Tubb3 mRNA remaining in Neuro2a cells in vitro 24 hours following treatment with various concentrations of lipid-conjugated Tubb3 oligonucleotides, as indicated, ranging from 100 nM to 100 pM. FIG. 9A provides results for lipid-conjugated blunt-end oligonucleotides (compared to the reference oligonucleotide) and FIG. 9B provides results for lipid-conjugated tetraloop oligonucleotides.

### DETAILED DESCRIPTION

In some aspects, the disclosure provides oligonucleotide-lipid conjugates (e.g., RNAi oligonucleotide-lipid conjugates) that reduce expression of a target gene expressed in neurons in the central nervous system (CNS). In other aspects, the disclosure provides methods of treating a disease or disorder associated with expression of a neuronal mRNA (e.g., a disease of the CNS). In other aspects, the disclosure provides methods of treating a disease or disorder (e.g., a neurological disease and/or by inappropriate gene expression) associated with expression of a neuronal mRNA using the lipid-conjugated RNAi oligonucleotides, or pharmaceutically acceptable compositions thereof, described herein. In other aspects, the disclosure provides methods of using the lipid-conjugated RNAi oligonucleotides described herein in the manufacture of a medicament for treating a disease or disorder associated with expression of a neuronal mRNA. In other aspects, the lipid-conjugated RNAi oligonucleotides provided herein are used to treat a neurological disease or disorder by modulating (e.g., inhibiting or reducing) expression of a neuronal target gene associated with the neurological disease or disorder in the CNS. In some aspects, the disclosure provides methods of treating a neurological disease or disorder by reducing expression of a neuronal target gene associated with the neurological disease or disorder in the CNS (e.g., in cells, tissues or regions of the CNS).

### Lipid-Conjugated RNAi Oligonucleotides

The disclosure provides, inter alia, lipid-conjugated RNAi oligonucleotides (e.g., RNAi oligonucleotide-lipid conjugates) that reduce expression of a neuronal target gene in the CNS. In some embodiments, a lipid-conjugated RNAi oligonucleotide provided by the disclosure is targeted to an mRNA encoding the target gene. Messenger RNA (mRNA) that encodes a target gene and is targeted by a lipid-conjugated RNAi oligonucleotide of the disclosure is referred to herein as “target mRNA”. In some embodiments, the lipid-conjugated RNAi oligonucleotide reduces target gene expression in the CNS (e.g., in the somatosensory cortex (SS cortex), hippocampus (HP), striatum, frontal cortex, cerebellum, medulla, hypothalamus (HY), cervical spinal cord (CSC), thoracic spinal cord (TSC), lumbar dorsal root ganglion (DRG), and/or lumbar spinal cord (LSC). In some embodiments, the lipid-conjugated RNAi oligonucleotide reduces target gene expression in the CNS (e.g., in the SS cortex, HP, HY, CSC, TSC, DRG, and/or LSC), without reducing expression of the target mRNA outside the CNS. In some embodiments, the lipid-conjugated RNAi oligonucleotide reduces target gene expression in the CNS (e.g., in the SS cortex, HP, HY, CSC, TSC, and/or LSC), without reducing expression of the target mRNA in the liver. In some embodiments, the lipid-conjugated RNAi oligonucleotide does not result in a reduction in the expression of the target mRNA in the liver to the same or similar level as in the CNS.

In some embodiments, the lipid-conjugated RNAi oligonucleotide reduces target gene expression in the CNS (e.g., in the somatosensory cortex (SS cortex), hippocampus (HP), frontal cortex, cerebellum, medulla, lumbar dorsal root ganglion (DRG), and/or lumbar spinal cord (LSC). In some embodiments, the lipid-conjugated RNAi oligonucleotide reduces target gene expression in the CNS (e.g., in the SS cortex, HP, frontal cortex, cerebellum, medulla DRG, and/or LSC), without reducing expression of the target mRNA outside the CNS. In some embodiments, the lipid-conjugated RNAi oligonucleotide reduces target gene expression in the CNS (e.g., in the SS cortex, HP, frontal cortex, cerebellum, medulla DRG, and/or LSC), without reducing expression of the target mRNA in the liver. In some embodiments, the lipid-conjugated RNAi oligonucleotide does not result in a reduction in the expression of the target mRNA in the liver to the same or similar level as in the CNS.

mRNA Target Sequences

In some embodiments, the lipid-conjugated RNAi oligonucleotide is targeted to a target sequence comprising a target neuronal mRNA. In some embodiments, the lipid-conjugated RNAi oligonucleotide is targeted to a target sequence within a target neuronal mRNA. In some embodiments, the lipid-conjugated RNAi oligonucleotide, or a portion, fragment, or strand thereof (e.g., an antisense strand or a guide strand of a double-stranded oligonucleotide) binds or anneals to a target sequence comprising a target neuronal mRNA, thereby reducing target gene expression. In some embodiments, the lipid-conjugated RNAi oligonucleotide is targeted to a target sequence comprising target neuronal mRNA for the purpose of reducing expression of a neuronal target gene in vivo. In some embodiments, the amount or extent of reduction of target gene expression by an lipid-conjugated RNAi oligonucleotide targeted to a specific neuronal target sequence correlates with the potency of the lipid-conjugated RNAi oligonucleotide. In some embodiments, the amount or extent of reduction of target gene expression by an lipid-conjugated RNAi oligonucleotide targeted to a specific neuronal target sequence correlates with the amount or extent of therapeutic benefit in a subject or patient having a disease, disorder or condition associated with target gene expression treated with the lipid-conjugated RNAi oligonucleotide.

Through examination of the nucleotide sequence of mRNAs encoding target genes, including mRNAs of multiple different species (e.g., human, cynomolgus monkey, mouse, and rat) and as a result of in vitro and in vivo testing, it has been discovered that certain nucleotide sequences and certain systemic modifications to those oligonucleotides are more amenable than others to RNAi oligonucleotide-mediated reduction and are thus useful as part of oligonucleotides that are otherwise targeted to specific gene target sequences. In some embodiments, a sense strand of a lipid-conjugated RNAi oligonucleotide, or a portion or fragment thereof, described herein, comprises a nucleotide sequence that is similar (e.g., having no more than 4 mismatches) or is identical to a target sequence comprising a neuronal target mRNA. In some embodiments, a portion or region of the sense strand of a double-stranded oligonucleotide described herein comprises a target sequence comprising a neuronal target mRNA.

In some embodiments, the neuronal mRNA target sequence is associated with acute or chronic pain. In some embodiments, the neuronal mRNA target sequence is associated with a neurological disorder. In some embodiments, the neuronal mRNA target sequence is an mRNA expressed in neurons of at least one region of the CNS. In some embodiments, the neuronal mRNA target sequence is an mRNA expressed in neurons of the spinal cord. In some embodiments, the neuronal mRNA target sequence is an mRNA expressed in neurons of the lumbar spinal cord. In some embodiments, the neuronal mRNA target sequence is an mRNA expressed in neurons of the thoracic spinal cord. In some embodiments, the neuronal mRNA target sequence is an mRNA expressed in neurons of the cervical spinal cord. In some embodiments, the neuronal mRNA target sequence is an mRNA expressed in neurons of the lumbar dorsal root ganglion. In some embodiments, the neuronal mRNA target sequence is an mRNA expressed in neurons of the medulla. In some embodiments, the neuronal mRNA target sequence is an mRNA expressed in neurons of the hippocampus. In some embodiments, the neuronal mRNA target sequence is an mRNA expressed in neurons of the somatosensory cortex. In some embodiments, the neuronal mRNA target sequence is an mRNA expressed in neurons of the frontal cortex. In some embodiments, the neuronal mRNA target sequence is an mRNA associated with a disease, disorder or condition of the CNS.

### RNAi Oligonucleotide Targeting Sequences

In some embodiments, the lipid-conjugated RNAi oligonucleotides provided by the disclosure comprise a targeting sequence. As used herein, the term “targeting sequence” refers to a nucleotide sequence having a region of complementarity to a specific nucleotide sequence comprising an mRNA (e.g., a neuronal target mRNA). In some embodiments, the lipid-conjugated RNAi oligonucleotides provided by the disclosure comprise a gene targeting sequence having a region of complementarity to a nucleotide sequence comprising a target sequence of a target mRNA. In some embodiments, the targeting sequence is a neuronal mRNA target sequence.

The targeting sequence imparts the lipid-conjugated RNAi oligonucleotide with the ability to specifically target an mRNA by binding or annealing to a target sequence comprising a target mRNA by complementary (Watson-Crick) base pairing. In some embodiments, the lipid-conjugated RNAi oligonucleotides herein (or a strand thereof, e.g., an antisense strand or a guide strand of a double-stranded oligonucleotide) comprise a targeting sequence having a region of complementarity that binds or anneals to a target sequence comprising a neuronal target mRNA by complementary (Watson-Crick) base pairing. In some embodiments, the lipid-conjugated RNAi oligonucleotides herein (or a strand thereof, e.g., an antisense strand or a guide strand of a double-stranded oligonucleotide) comprise a targeting sequence having a region of complementarity that binds or anneals to a target sequence within a neuronal target mRNA by complementary (Watson-Crick) base pairing. The targeting sequence is generally of suitable length and base content to enable binding or annealing of the lipid-conjugated RNAi oligonucleotide (or a strand thereof) to a specific target mRNA (e.g., neuronal mRNA) for purposes of inhibiting target gene expression. In some embodiments, the targeting sequence is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence is at least 12, at least 13, at least 14, at least 15, at least 16, at least 17, at least 18, at least 19 or at least 20 nucleotides. In some embodiments, the targeting sequence is about 12 to about 30 (e.g., 12 to 30, 12 to 22, 15 to 25, 17 to 21, 18 to 27, 19 to 27, or 15 to 30) nucleotides in length. In some embodiments, the targeting sequence is about 12, 13, 14, 15, 16, 17, 18, 19, 20, 21, 22, 23, 24, 25, 26, 27, 28, 29 or 30 nucleotides in length. In some embodiments, the targeting sequence is 18 nucleotides in length. In some embodiments, the targeting sequence is 19 nucleotides in length. In some embodiments, the targeting sequence is 20 nucleotides in length. In some embodiments, the targeting sequence is 21 nucleotides in length. In some embodiments, the targeting sequence is 22 nucleotides in length. In some embodiments, the targeting sequence is 23 nucleotides in length. In some embodiments, the targeting sequence is 24 nucleotides in length.

In some embodiments, the lipid-conjugated RNAi oligonucleotides herein comprise a targeting sequence that is fully complementary to a target sequence comprising a neuronal target mRNA. In some embodiments, the lipid-conjugated RNAi oligonucleotides herein comprise a targeting sequence that is fully complementary to a target sequence within a neuronal target mRNA. In some embodiments, the targeting sequence is partially complementary to a target sequence comprising a target mRNA. In some embodiments, the targeting sequence is partially complementary to a target sequence within a neuronal target mRNA. In some embodiments, the targeting sequence comprises a region of contiguous nucleotides comprising the antisense strand.

In some embodiments, the lipid-conjugated RNAi oligonucleotides herein comprise a targeting sequence that is complementary to a contiguous sequence of nucleotides comprising a neuronal target mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the lipid-conjugated RNAi oligonucleotides comprise a targeting sequence that is complementary to a contiguous sequence of nucleotides comprising a neuronal target mRNA, wherein the contiguous sequence of nucleotides is 10, 11, 12, 13, 14, 15, 16, 17, 18, 19 or 20 nucleotides in length. In some embodiments, the lipid-conjugated RNAi oligonucleotides comprise a targeting sequence that is complementary to a contiguous sequence of nucleotides comprising a target mRNA, wherein the contiguous sequence of nucleotides is 15 nucleotides in length. In some embodiments, the lipid-conjugated RNAi oligonucleotides comprise a targeting sequence that is complementary to a contiguous sequence of nucleotides comprising a target mRNA, wherein the contiguous sequence of nucleotides is 19 nucleotides in length.

In some embodiments, the lipid-conjugated RNAi oligonucleotide comprises a targeting sequence that is complementary to a contiguous sequence of nucleotides comprising a neuronal target mRNA, wherein the contiguous sequence of nucleotides is 15 nucleotides in length. In some embodiments, the lipid-conjugated RNAi oligonucleotide comprises a targeting sequence that is complementary to a contiguous sequence of nucleotides comprising a neuronal target mRNA, wherein the contiguous sequence of nucleotides is 19 nucleotides in length.

In some embodiments, a targeting sequence of a lipid-conjugated RNAi oligonucleotide herein is fully complementary (e.g., having no mismatches) to a target sequence comprising a neuronal target mRNA and comprises the entire length of an antisense strand. In some embodiments, a targeting sequence of a lipid-conjugated RNAi oligonucleotide herein is fully complementary (e.g., having no mismatches) to a target sequence comprising a neuronal target mRNA and comprises a portion of the entire length of an antisense strand. In some embodiments, a targeting sequence of a lipid-conjugated RNAi oligonucleotide herein is fully complementary (e.g., having no mismatches) to a target sequence comprising a neuronal target mRNA and comprises 10 to 20 nucleotides of the antisense strand. In some embodiments, a targeting sequence of a lipid-conjugated RNAi oligonucleotide herein is fully complementary (e.g., having no mismatches) to a target sequence comprising a neuronal target mRNA and comprises 15 to 19 nucleotides of the antisense strand. In some embodiments, a targeting sequence of a lipid-conjugated RNAi oligonucleotide herein is fully complementary (e.g., having no mismatches) to a target sequence comprising a neuronal target mRNA and comprises 15 nucleotides, 16 nucleotides, 17 nucleotides, 18 nucleotides, 19 nucleotides, 20 nucleotides, 21 nucleotides, or 22 nucleotides of the antisense strand. In some embodiments, a targeting sequence of a lipid-conjugated RNAi oligonucleotide herein is fully complementary (e.g., having no mismatches) to a target sequence comprising a neuronal target mRNA and comprises 19 nucleotides of the antisense strand. In some embodiments, a targeting sequence of a lipid-conjugated RNAi oligonucleotide herein is fully complementary (e.g., having no mismatches) to a target sequence comprising a neuronal target mRNA and comprises 20 nucleotides of the antisense strand.

In some embodiments, a targeting sequence of a lipid-conjugated RNAi oligonucleotide herein is partially complementary (e.g., having no more than 4 mismatches) to a target sequence comprising a neuronal target mRNA and comprises the entire length of an antisense strand. In some embodiments, a targeting sequence of a lipid-conjugated RNAi oligonucleotide herein is partially complementary (e.g., having no more than 4 mismatches) to a target sequence comprising a neuronal target mRNA and comprises a portion of the entire length of an antisense strand. In some embodiments, a targeting sequence of a lipid-conjugated RNAi oligonucleotide herein is partially complementary (e.g., having no more than 4 mismatches) to a target sequence comprising a neuronal target mRNA and comprises 10 to 20 nucleotides of the antisense strand. In some embodiments, a targeting sequence of a lipid-conjugated RNAi oligonucleotide herein is partially complementary (e.g., having no more than 4 mismatches) to a target sequence comprising a neuronal target mRNA and comprises 15 to 19 nucleotides of the antisense strand. In some embodiments, a targeting sequence of a lipid-conjugated RNAi oligonucleotide herein is partially complementary (e.g., having no more than 4 mismatches) to a target sequence comprising a neuronal target mRNA and comprises 15 nucleotides, 16 nucleotides, 17 nucleotides, 18 nucleotides, 19 nucleotides, 20 nucleotides, 21 nucleotides, or 22 nucleotides of the antisense strand. In some embodiments, a targeting sequence of a lipid-conjugated RNAi oligonucleotide herein is partially complementary (e.g., having no more than 4 mismatches) to a target sequence comprising a neuronal target mRNA and comprises 19 nucleotides of the antisense strand. In some embodiments, a targeting sequence of a lipid-conjugated RNAi oligonucleotide herein is partially complementary (e.g., having no more than 4 mismatches) to a target sequence comprising a neuronal target mRNA and comprises 20 nucleotides of the antisense strand.

In some embodiments, a lipid-conjugated RNAi oligonucleotide herein comprises a targeting sequence having one or more base pair (bp) mismatches with the corresponding target sequence comprising a neuronal target mRNA. In some embodiments, the targeting sequence has a 1 bp mismatch, a 2 bp mismatch, a 3 bp mismatch, a 4 bp mismatch, or a 5 bp mismatch with the corresponding target sequence comprising a neuronal target mRNA provided that the ability of the targeting sequence to bind or anneal to the target sequence under appropriate hybridization conditions and/or the ability of the lipid-conjugated RNAi oligonucleotide to inhibit or reduce target gene expression is maintained (e.g., under physiological conditions). Alternatively, in some embodiments, the targeting sequence comprises no more than 1, no more than 2, no more than 3, no more than 4, or no more than 5 bp mismatches with the corresponding target sequence comprising a neuronal target mRNA provided that the ability of the targeting sequence to bind or anneal to the target sequence under appropriate hybridization conditions and/or the ability of the lipid-conjugated RNAi oligonucleotide to inhibit or reduce target gene expression is maintained. In some embodiments, the lipid-conjugated RNAi oligonucleotide comprises a targeting sequence having 1 mismatch with the corresponding target sequence. In some embodiments, the lipid-conjugated RNAi oligonucleotide comprises a targeting sequence having 2 mismatches with the corresponding target sequence. In some embodiments, the lipid-conjugated RNAi oligonucleotide comprises a targeting sequence having 3 mismatches with the corresponding target sequence. In some embodiments, the lipid-conjugated RNAi oligonucleotide comprises a targeting sequence having 4 mismatches with the corresponding target sequence. In some embodiments, the lipid-conjugated RNAi oligonucleotide comprises a targeting sequence having 5 mismatches with the corresponding target sequence. In some embodiments, the lipid-conjugated RNAi oligonucleotide comprises a targeting sequence having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein the mismatches are interspersed in any position throughout the targeting sequence. In some embodiments, the lipid-conjugated RNAi oligonucleotide comprises a targeting sequence having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof.

### Types of Oligonucleotides

A variety of RNAi oligonucleotide types and/or structures are useful for reducing target gene expression (e.g., reducing expression of a target gene expressed in a neuron) in the methods herein. Any of the RNAi oligonucleotide types described herein or elsewhere are contemplated for use as a framework to incorporate a targeting sequence herein for the purposes of inhibiting or reducing corresponding target gene expression in a neuron in the CNS.

In some embodiments, the lipid-conjugated RNAi oligonucleotides herein inhibit target gene expression by engaging with RNA interference (RNAi) pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as Intl. Patent Application Publication No. WO 2010/033225). Such structures may include single-stranded extensions (on one or both sides of the molecule) as well as double-stranded extensions.

In some embodiments, the RNAi oligonucleotides conjugates herein engage with the RNAi pathway downstream of the involvement of Dicer (e.g., Dicer cleavage). In some embodiments, the oligonucleotides described herein are Dicer substrates. In some embodiments, upon endogenous Dicer processing, double-stranded nucleic acids of 19-23 nucleotides in length capable of reducing expression of a neuronal target mRNA are produced. In some embodiments, the lipid-conjugated RNAi oligonucleotide has an overhang (e.g., of 1, 2, or 3 nucleotides in length) in the 3′ end of the sense strand. In some embodiments, the lipid-conjugated RNAi oligonucleotide (e.g., siRNA conjugate) comprises a 21-nucleotide guide strand that is antisense to a neuronal target mRNA and a complementary passenger strand, in which both strands anneal to form a 19-bp duplex and 2 nucleotide overhangs at either or both 3′ ends. Longer oligonucleotide designs also are contemplated including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a 21 bp duplex region. See, e.g., U.S. Pat. Nos. 9,012,138; 9,012,621 and 9,193,753.

In some embodiments, the RNAi oligonucleotides conjugates disclosed herein comprise sense and antisense strands that are both in the range of about 17 to 26 (e.g., 17 to 26, 20 to 25 or 21-23) nucleotides in length. In some embodiments, the lipid-conjugated RNAi oligonucleotides disclosed herein comprise a sense and antisense strand that are both in the range of about 19-22 nucleotides in length. In some embodiments, the sense and antisense strands are of equal length. In some embodiments, the lipid-conjugated RNAi oligonucleotides disclosed herein comprise sense and antisense strands, such that there is a 3′-overhang on either the sense strand or the antisense strand, or both the sense and antisense strand. In some embodiments, for lipid-conjugated RNAi oligonucleotides that have sense and antisense strands that are both in the range of about 21-23 nucleotides in length, a 3′ overhang on the sense, antisense, or both sense and antisense strands is 1 or 2 nucleotides in length. In some embodiments, an lipid-conjugated RNAi oligonucleotide has a guide strand of 22 nucleotides and a passenger strand of 20 nucleotides, where there is a blunt end on the right side of the molecule (3′ end of passenger strand/5′ end of guide strand) and a 2 nucleotide 3′-guide strand overhang on the left side of the molecule (5′ end of the passenger strand/3′ end of the guide strand). In such molecules, there is a 20 bp duplex region.

Other RNAi oligonucleotide designs for use with the compositions and methods herein include: 16-mer siRNAs (see, e.g., Nucleic Acids in Chemistry and Biology, Blackburn (ed.), ROYAL SOCIETY OF CHEMISTRY, 2006), shRNAs (e.g., having 19 bp or shorter stems; see, e.g., Moore et al. (2010) METHODS MOL. BIOL. 629:141-58), blunt siRNAs (e.g., of 19 bps in length; see, e.g., Kraynack & Baker (2006) RNA 12:163-76), asymmetrical siRNAs (aiRNA; see, e.g., Sun et al. (2008) NAT. BIOTECHNOL. 26:1379-82), asymmetric shorter-duplex siRNA (see, e.g., Chang et al. (2009) MOL. THER. 17:725-32), fork siRNAs (see, e.g., Hohjoh (2004) FEBS Lett. 557:193-98), and small internally segmented interfering RNA (siRNA; see, e.g., Bramsen et al. (2007) NUCLEIC ACIDS RES. 35:5886-97). Further non-limiting examples of an oligonucleotide structure that may be used in some embodiments to reduce or inhibit the expression of a target gene are microRNA (miRNA), short hairpin RNA (shRNA) and short siRNA (see, e.g., Hamilton et al. (2002) EMBO J. 21:4671-79; see also, US Patent Application Publication No. 2009/0099115).

### Antisense Strands

In some embodiments, an antisense strand of a lipid-conjugated RNAi oligonucleotide is referred to as a “guide strand.” For example, an antisense strand that engages with RNA-induced silencing complex (RISC) and binds to an Argonaute protein such as Ago2, or engages with or binds to one or more similar factors, and directs silencing of a target gene, the antisense strand is referred to as a guide strand. In some embodiments, a sense strand complementary to a guide strand is referred to as a “passenger strand.”

In some embodiments, a lipid-conjugated RNAi oligonucleotide herein comprises an antisense strand of up to about 50 nucleotides in length (e.g., up to 50, up to 40, up to 35, up to 30, up to 27, up to 25, up to 21, up to 19, up to 17, up to 15, or up to 12 nucleotides in length). In some embodiments, a lipid-conjugated RNAi oligonucleotide herein comprises an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, a herein comprises an antisense strand in a range of about 12 to about 40 (e.g., 12 to 40, 12 to 36, 12 to 32, 12 to 28, 15 to 40, 15 to 36, 15 to 32, 15 to 30, 15 to 28, 17 to 22, 17 to 25, 19 to 27, 19 to 30, 20 to 40, 22 to 40, 25 to 40 or 32 to 40) nucleotides in length. In some embodiments, a lipid-conjugated RNAi oligonucleotide herein comprises an antisense strand of 15 to 30 nucleotides in length. In some embodiments, an antisense strand of any one of the lipid-conjugated RNAi oligonucleotide disclosed herein is of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, a lipid-conjugated RNAi oligonucleotide comprises an antisense strand of 19-23 nucleotides in length. In some embodiments, a lipid-conjugated RNAi oligonucleotide comprises an antisense strand of 19 nucleotides in length. In some embodiments, a lipid-conjugated RNAi oligonucleotide comprises an antisense strand of 20 nucleotides in length. In some embodiments, a lipid-conjugated RNAi oligonucleotide comprises an antisense strand of 21 nucleotides in length. In some embodiments, a lipid-conjugated RNAi oligonucleotide comprises an antisense strand of 22 nucleotides in length. In some embodiments, a lipid-conjugated RNAi oligonucleotide comprises an antisense strand of 23 nucleotides in length.

### Sense Strands

In some embodiments, a lipid-conjugated RNAi oligonucleotide disclosed herein comprises a sense strand (or passenger strand) of up to about 50 nucleotides in length (e.g., up to 50, up to 40, up to 36, up to 30, up to 27, up to 25, up to 21, up to 19, up to 17 or up to 12 nucleotides in length). In some embodiments, a lipid-conjugated RNAi oligonucleotide herein comprises a sense strand of at least about 12 nucleotides in length (e.g., at least 12, at least 15, at least 19, at least 21, at least 25, at least 27, at least 30, at least 36 or at least 38 nucleotides in length). In some embodiments, a lipid-conjugated RNAi oligonucleotide herein comprises a sense strand in a range of about 12 to about 50 (e.g., 12 to 50, 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, a lipid-conjugated RNAi oligonucleotide herein comprises a sense strand 15 to 50 nucleotides in length. In some embodiments, a lipid-conjugated RNAi oligonucleotide herein comprises a sense strand 18 to 36 nucleotides in length. In some embodiments, a lipid-conjugated RNAi oligonucleotide herein comprises a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, a lipid-conjugated RNAi oligonucleotide herein comprises a sense strand of 17-21 nucleotides in length. In some embodiments, a lipid-conjugated RNAi oligonucleotide herein comprises a sense strand of 17 nucleotides in length. In some embodiments, a lipid-conjugated RNAi oligonucleotide herein comprises a sense strand of 18 nucleotides in length. In some embodiments, a lipid-conjugated RNAi oligonucleotide herein comprises a sense strand of 19 nucleotides in length. In some embodiments, a lipid-conjugated RNAi oligonucleotide herein comprises a sense strand of 20 nucleotides in length. In some embodiments, a lipid-conjugated RNAi oligonucleotide herein comprises a sense strand of 21 nucleotides in length. In some embodiments, a lipid-conjugated RNAi oligonucleotide herein comprises a sense strand of 22 nucleotides in length. In some embodiments, a lipid-conjugated RNAi oligonucleotide herein comprises a sense strand of 23 nucleotides in length. In some embodiments, a lipid-conjugated RNAi oligonucleotide herein comprises a sense strand of 24 nucleotides in length. In some embodiments, a lipid-conjugated RNAi oligonucleotide herein comprises a sense strand of 25 nucleotides in length. In some embodiments, a lipid-conjugated RNAi oligonucleotide herein comprises a sense strand of 26 nucleotides in length. In some embodiments, a lipid-conjugated RNAi oligonucleotide herein comprises a sense strand of 27 nucleotides in length. In some embodiments, a lipid-conjugated RNAi oligonucleotide herein comprises a sense strand of 28 nucleotides in length. In some embodiments, a lipid-conjugated RNAi oligonucleotide herein comprises a sense strand of 29 nucleotides in length. In some embodiments, a lipid-conjugated RNAi oligonucleotide herein comprises a sense strand of 30 nucleotides in length. In some embodiments, a lipid-conjugated RNAi oligonucleotide herein comprises a sense strand of 31 nucleotides in length. In some embodiments, a lipid-conjugated RNAi oligonucleotide herein comprises a sense strand of 32 nucleotides in length. In some embodiments, a lipid-conjugated RNAi oligonucleotide herein comprises a sense strand of 33 nucleotides in length. In some embodiments, a lipid-conjugated RNAi oligonucleotide herein comprises a sense strand of 34 nucleotides in length. In some embodiments, a lipid-conjugated RNAi oligonucleotide herein comprises a sense strand of 35 nucleotides in length. In some embodiments, a lipid-conjugated RNAi oligonucleotide herein comprises a sense strand of 36 nucleotides in length.

In some embodiments, a sense strand comprises a stem-loop structure at its 3′ end. In some embodiments, the stem-loop is formed by intrastrand base pairing. In some embodiments, a sense strand comprises a stem-loop structure at its 5′ end. In some embodiments, a stem is a duplex of 2, 3, 4, 5, 6, 7, 8, 9, 10, 11, 12, 13 or 14 nucleotides in length. In some embodiments, the stem of the stem-loop comprises a duplex of 2 nucleotides in length. In some embodiments, the stem of the stem-loop comprises a duplex of 3 nucleotides in length. In some embodiments, the stem of the stem-loop comprises a duplex of 4 nucleotides in length. In some embodiments, the stem of the stem-loop comprises a duplex of 5 nucleotides in length. In some embodiments, the stem of the stem-loop comprises a duplex of 6 nucleotides in length. In some embodiments, the stem of the stem-loop comprises a duplex of 7 nucleotides in length. In some embodiments, the stem of the stem-loop comprises a duplex of 8 nucleotides in length. In some embodiments, the stem of the stem-loop comprises a duplex of 9 nucleotides in length. In some embodiments, the stem of the stem-loop comprises a duplex of 10 nucleotides in length. In some embodiments, the stem of the stem-loop comprises a duplex of 11 nucleotides in length. In some embodiments, the stem of the stem-loop comprises a duplex of 12 nucleotides in length. In some embodiments, the stem of the stem-loop comprises a duplex of 13 nucleotides in length. In some embodiments, the stem of the stem-loop comprises a duplex of 14 nucleotides in length.

In some embodiments, a stem-loop provides the lipid-conjugated RNAi oligonucleotide protection against degradation (e.g., enzymatic degradation), facilitates or improves targeting and/or delivery to a target cell, tissue, or organ, or both. For example, in some embodiments, the loop of a stem-loop provides nucleotides comprising one or more modifications that facilitate, improve, or increase targeting to a target mRNA (e.g., a target mRNA expressed in the CNS), inhibition of target gene expression, and/or delivery to a target cell, tissue, or organ (e.g., the CNS), or a combination thereof. In some embodiments, the stem-loop itself or modification(s) to the stem-loop do not substantially affect the inherent gene expression inhibition activity of the lipid-conjugated RNAi oligonucleotide, but facilitates, improves, or increases stability (e.g., provides protection against degradation) and/or delivery of the lipid-conjugated RNAi oligonucleotide to a target cell, tissue, or organ (e.g., the CNS). In certain embodiments, a lipid-conjugated RNAi oligonucleotide herein comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the loop (L) is 3 nucleotides in length. In some embodiments, the loop (L) is 4 nucleotides in length. In some embodiments, the loop (L) is 5 nucleotides in length. In some embodiments, the loop (L) is 6 nucleotides in length. In some embodiments, the loop (L) is 7 nucleotides in length. In some embodiments, the loop (L) is 8 nucleotides in length. In some embodiments, the loop (L) is 9 nucleotides in length. In some embodiments, the loop (L) is 10 nucleotides in length.

In some embodiments, the tetraloop comprises the sequence 5′-GAAA-3′. In some embodiments, the stem loop comprises the sequence 5′-GCAGCCGAAAGGCUGC-3′ (SEQ ID NO: 21).

In some embodiments, a loop (L) of a stem-loop having the structure S1-L-S2 as described above is a triloop. In some embodiments, the triloop comprises ribonucleotides, deoxyribonucleotides, modified nucleotides, delivery ligands, and combinations thereof.

In some embodiments, a loop (L) of a stem-loop having the structure S1-L-S2 as described above is a tetraloop (e.g., within a nicked tetraloop structure). In some embodiments, the tetraloop comprises ribonucleotides, deoxyribonucleotides, modified nucleotides, delivery ligands, and combinations thereof.

In some embodiments, a loop (L) of a stem-loop having the structure S1-L-S2 as described above is a tetraloop as described in U.S. Pat. No. 10,131,912, incorporated herein by reference (e.g., within a nicked tetraloop structure).

### Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 29, 21, 22, 23, 24, 25, 26, 27, 28, 29 or 30 nucleotides in length. In some embodiments, a duplex formed between a sense and antisense strand is 15-30 base pairs in length. In some embodiments, a duplex formed between a sense and antisense strand is 17-21 base pairs in length. In some embodiments, a duplex formed between a sense and antisense strand is 17 base pairs in length. In some embodiments, a duplex formed between a sense and antisense strand is 18 base pairs in length. In some embodiments, a duplex formed between a sense and antisense strand is 19 base pairs in length. In some embodiments, a duplex formed between a sense and antisense strand is 20 base pairs in length. In some embodiments, a duplex formed between a sense and antisense strand is 21 base pairs in length. In some embodiments, a duplex formed between a sense and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand.

In some embodiments, there is one or more (e.g., 1, 2, 3, 4 or 5) mismatch(s) between a sense and antisense strand. If there is more than one mismatch between a sense and antisense strand, they may be positioned consecutively (e.g., 2, 3 or more in a row), or interspersed throughout the region of complementarity. In some embodiments, the 3′ end of the sense strand contains one or more mismatches. In one embodiment, two mismatches are incorporated at the 3′ end of the sense strand. In some embodiments, base mismatches, or destabilization of segments at the 3′ end of the sense strand of an oligonucleotide-ligand conjugate herein improves or increases the potency and/or efficacy of the oligonucleotide-ligand conjugate.

### Oligonucleotide Ends

In some embodiments, a lipid-conjugated RNAi oligonucleotide disclosed herein comprises sense and antisense strands, such that there is a 3′-overhang on either the sense strand or the antisense strand, or both the sense and antisense strand. In some embodiments, a lipid-conjugated RNAi oligonucleotide herein has one 5′end that is thermodynamically less stable compared to the other 5′ end. In some embodiments, an asymmetric lipid-conjugated RNAi oligonucleotide conjugate is provided that includes a blunt end at the 3′end of a sense strand and overhang at the 3′ end of the antisense strand. In some embodiments, a 3′ overhang on an antisense strand is 1-4 nucleotides in length (e.g., 1, 2, 3, or 4 nucleotides in length).

In some embodiments, the 3′-overhang is about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length). In some embodiments, the 3′ overhang is about one (1) to nineteen (19), one (1) to eighteen (18), one (1) to seventeen (17), one (1) to sixteen (16), one (1) to fifteen (15), one (1) to fourteen (14), one (1) to thirteen (13), one (1) to twelve (12), one (1) to eleven (11), one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 3′-overhang is (1) nucleotide in length. In some embodiments, the 3′-overhang is two (2) nucleotides in length. In some embodiments, the 3′-overhang is three (3) nucleotides in length. In some embodiments, the 3′-overhang is four (4) nucleotides in length. In some embodiments, the 3′-overhang is five (5) nucleotides in length. In some embodiments, the 3′-overhang is six (6) nucleotides in length. In some embodiments, the 3′-overhang is seven (7) nucleotides in length. In some embodiments, the 3′-overhang is eight (8) nucleotides in length. In some embodiments, the 3′-overhang is nine (9) nucleotides in length. In some embodiments, the 3′-overhang is ten (10) nucleotides in length. In some embodiments, the 3′-overhang is eleven (11) nucleotides in length. In some embodiments, the 3′-overhang is twelve (12) nucleotides in length. In some embodiments, the 3′-overhang is thirteen (13) nucleotides in length. In some embodiments, the 3′-overhang is fourteen (14) nucleotides in length. In some embodiments, the 3′-overhang is fifteen (15) nucleotides in length. In some embodiments, the 3′-overhang is sixteen (16) nucleotides in length. In some embodiments, the 3′-overhang is seventeen (17) nucleotides in length. In some embodiments, the 3′-overhang is eighteen (18) nucleotides in length. In some embodiments, the 3′-overhang is nineteen (19) nucleotides in length. In some embodiments, the 3′-overhang is twenty (20) nucleotides in length.

Typically, an oligonucleotide for RNAi has a two (2) nucleotide overhang on the 3′ end of the antisense (guide) strand. However, other overhangs are possible. In some embodiments, an overhang is a 3′ overhang comprising a length of between one and four nucleotides, optionally one to four, one to three, one to two, two to four, two to three, or one, two, three, or four nucleotides. In some embodiments, the overhang is a 5′ overhang comprising a length of between one and four nucleotides, optionally one to four, one to three, one to two, two to four, two to three, or one, two, three, or four nucleotides.

In some embodiments, an oligonucleotide herein comprises a sense strand and an antisense strand, wherein the 5′ terminus of either or both strands comprise a 5′-overhang comprising one or more nucleotides. In some embodiments, an oligonucleotide herein comprises a sense strand and an antisense strand, wherein the sense strand comprises a 5′-overhang comprising one or more nucleotides. In some embodiments, an oligonucleotide herein comprises a sense strand and an antisense strand, wherein the antisense strand comprises a 5′-overhang comprising one or more nucleotides. In some embodiments, an oligonucleotide herein comprises a sense strand and an antisense strand, wherein both the sense strand and the antisense strand comprises a 5′-overhang comprising one or more nucleotides.

In some embodiments, the 5′-overhang is about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length). In some embodiments, the 5′ overhang is about one (1) to nineteen (19), one (1) to eighteen (18), one (1) to seventeen (17), one (1) to sixteen (16), one (1) to fifteen (15), one (1) to fourteen (14), one (1) to thirteen (13), one (1) to twelve (12), one (1) to eleven (11), one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 5′-overhang is (1) nucleotide in length. In some embodiments, the 5′-overhang is two (2) nucleotides in length. In some embodiments, the 5′-overhang is three (3) nucleotides in length. In some embodiments, the 5′-overhang is four (4) nucleotides in length. In some embodiments, the 5′-overhang is five (5) nucleotides in length. In some embodiments, the 5′-overhang is six (6) nucleotides in length. In some embodiments, the 5′-overhang is seven (7) nucleotides in length. In some embodiments, the 5′-overhang is eight (8) nucleotides in length. In some embodiments, the 5′-overhang is nine (9) nucleotides in length. In some embodiments, the 5′-overhang is ten (10) nucleotides in length. In some embodiments, the 5′-overhang is eleven (11) nucleotides in length. In some embodiments, the 5′-overhang is twelve (12) nucleotides in length. In some embodiments, the 5′-overhang is thirteen (13) nucleotides in length. In some embodiments, the 5′-overhang is fourteen (14) nucleotides in length. In some embodiments, the 5′-overhang is fifteen (15) nucleotides in length. In some embodiments, the 5′-overhang is sixteen (16) nucleotides in length. In some embodiments, the 5′-overhang is seventeen (17) nucleotides in length. In some embodiments, the 5′-overhang is eighteen (18) nucleotides in length. In some embodiments, the 5′-overhang is nineteen (19) nucleotides in length. In some embodiments, the 5′-overhang is twenty (20) nucleotides in length.

In some embodiments, one or more (e.g., 2, 3, or 4) terminal nucleotides of the 3′ end or 5′ end of a sense and/or antisense strand are modified. For example, in some embodiments, one or two terminal nucleotides of the 3′ end of the antisense strand are modified. In some embodiments, the last nucleotide at the 3′ end of an antisense strand is modified, e.g., comprises 2′ modification, e.g., a 2′-O-methoxyethyl. In some embodiments, the last one or two terminal nucleotides at the 3′ end of an antisense strand are complementary with the target. In some embodiments, the last one or two nucleotides at the 3′ end of the antisense strand are not complementary with the target.

In some embodiments, an RNAi oligonucleotide conjugate disclosed herein comprises a stem-loop structure at the 3′ end of the sense strand and comprises two terminal overhang nucleotides at the 3′ end of the antisense strand. In some embodiments, an RNAi oligonucleotide conjugate herein comprises a nicked tetraloop structure, wherein the 3′ end of the sense strand comprises a stem-tetraloop structure and comprises two terminal overhang nucleotides at the 3′ end of the antisense strand.

In some embodiments, the overhang is selected from AA, GG, AG, and GA. In some embodiments, the overhang is AA. In some embodiments, the overhang is AG. In some embodiments, the overhang is GA. In some embodiments, the two terminal overhang nucleotides are GG. Typically, one or both of the two terminal GG nucleotides of the antisense strand are not complementary with the target.

In some embodiments, the 5′ end and/or the 3′end of a sense or antisense strand has an inverted cap nucleotide.

In some embodiments, one or more (e.g., 2, 3, 4, 5, 6) modified internucleotide linkages are provided between terminal nucleotides of the 3′ end or 5′ end of a sense and/or antisense strand. In some embodiments, modified internucleotide linkages are provided between overhang nucleotides at the 3′ end or 5′ end of a sense and/or antisense strand.

### Oligonucleotide Modifications

In some embodiments, an RNAi oligonucleotide conjugate disclosed herein comprises one or more modifications. Oligonucleotides (e.g., RNAi oligonucleotides) may be modified in various ways to improve or control specificity, stability, delivery, bioavailability, resistance from nuclease degradation, immunogenicity, base-pairing properties, RNA distribution and cellular uptake and other features relevant to therapeutic research use.

In some embodiments, the modification is a modified sugar. In some embodiments, the modification is a 5′-terminal phosphate group. In some embodiments, the modification is a modified internucleoside linkage. In some embodiments, the modification is a modified base. In some embodiments, an oligonucleotide described herein can comprise any one of the modifications described herein or any combination thereof. For example, in some embodiments, an oligonucleotide described herein comprises at least one modified sugar, a 5′-terminal phosphate group, at least one modified internucleoside linkage, and at least one modified base.

The number of modifications on an oligonucleotide (e.g., an RNAi oligonucleotide) and the position of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of the nucleotides to be modified. Accordingly, in some embodiments, all or substantially all of the nucleotides of an oligonucleotides are modified. In some embodiments, more than half of the nucleotides are modified. In some embodiments, less than half of the nucleotides are modified. In some embodiments, the sugar moiety of all nucleotides comprising the oligonucleotide is modified at the 2′ position. In some embodiments, the sugar moiety of all nucleotides comprising the oligonucleotide is modified at the 2′ position, except for the nucleotide conjugated to a lipid (e.g., the 5′-terminal nucleotide of the sense strand). The modifications may be reversible or irreversible. In some embodiments, an oligonucleotide as disclosed herein has a number and type of modified nucleotides sufficient to cause the desired characteristics (e.g., protection from enzymatic degradation, capacity to target a desired cell after in vivo administration, and/or thermodynamic stability).

### Sugar Modifications

In some embodiments, a nucleotide modification in a sugar comprises a 2′-modification. In some embodiments, a 2′-modification may be 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O-[2-(methylamino)-2-oxoethyl] (2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, a lipid-conjugated RNAi oligonucleotide described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the lipid-conjugated RNAi oligonucleotide comprises at least about 1 modified nucleotide (e.g., at least 1, at least 5, at least 10, at least 15, at least 20, at least 25, at least 30, at least 35, or more). In some embodiments, the antisense strand of the lipid-conjugated RNAi oligonucleotide comprises at least about 1 modified nucleotide (e.g., at least 1, at least 5, at least 10, at least 15, at least 20, or more).

In some embodiments, all the nucleotides of the sense strand of the lipid-conjugated RNAi oligonucleotide are modified. In some embodiments, all the nucleotides of the antisense strand of the lipid-conjugated RNAi oligonucleotide are modified. In some embodiments, all the nucleotides of the lipid-conjugated RNAi oligonucleotide (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-β-d-arabinonucleic acid).

In some embodiments, the disclosure provides lipid-conjugated RNAi oligonucleotides having different modification patterns. In some embodiments, the modified lipid-conjugated RNAi oligonucleotides comprise a sense strand sequence having a modification pattern as set forth in the Examples and Sequence Listing and an antisense strand having a modification pattern as set forth in the Examples and Sequence Listing.

In some embodiments, a lipid-conjugated RNAi oligonucleotide disclosed herein comprises an antisense strand having nucleotides that are modified with 2′-F. In some embodiments, an lipid-conjugated RNAi oligonucleotide disclosed herein comprises an antisense strand comprises nucleotides that are modified with 2′-F and 2′-OMe. In some embodiments, a lipid-conjugated RNAi oligonucleotide disclosed herein comprises a sense strand having nucleotides that are modified with 2′-F. In some embodiments, a lipid-conjugated RNAi oligonucleotide disclosed herein comprises a sense strand comprises nucleotides that are modified with 2′-F and 2′-OMe.

In some embodiments, an oligonucleotide described herein comprises a sense strand with about 10-25%, 10%, 11%, 12%, 13%, 14% 15%, 16%, 17%, 18%, 19% or 20% of the nucleotides of the sense strand comprising a 2′-fluoro modification. In some embodiments, about 11% of the nucleotides of the sense strand comprise a 2-fluoro modification. In some embodiments, about 20% of the nucleotides of the sense strand comprise a 2-fluoro modification. In some embodiments, an oligonucleotide described herein comprises an antisense strand with about 25-35%, 25%, 26%, 27%, 28%, 29%, 30%, 31%, 32%, 33%, 34% or 35% of the nucleotides of the antisense strand comprising a 2′-fluoro modification. In some embodiments, about 32% of the nucleotides of the antisense strand comprise a 2′-fluoro modification. In some embodiments, the oligonucleotide has about 1⁵-2⁵%, 15%, 16%, 17%, 18%, 19%, 20%, 21%, 22%, 23%, 24%, or 25% of its nucleotides comprising a 2′-fluoro modification. In some embodiments, about 19% of the nucleotides in the oligonucleotide comprise a 2′-fluoro modification. In some embodiments, about 26% of the nucleotides in the oligonucleotide comprise a 2′-fluoro modification.

In some embodiments, for these oligonucleotides, one or more of positions 8, 9, 10 or 11 of the sense strand is modified with a 2′-F group. In some embodiments, for these oligonucleotides, the sugar moiety at each of nucleotides not modified with a 2′-F group or conjugated to a lipid in the sense strand is modified with a 2′-OMe. In some embodiments, for these oligonucleotides, the sugar moiety at each of nucleotides at positions 1-7 and 12-20 in the sense strand is modified with a 2′-OMe. In some embodiments, for these oligonucleotides, the sugar moiety at each of nucleotides at positions 2-7 and 12-20 in the sense strand is modified with a 2′-OMe. In some embodiments, for these oligonucleotides, the sugar moiety at each of nucleotides at positions 1-6 and 12-20 in the sense strand is modified with a 2′-OMe. In some embodiments, for these oligonucleotides the sugar moiety at each of nucleotides at positions 1-7 and 12-36 in the sense strand is modified with a 2′-OMe. In some embodiments, for these oligonucleotides the sugar moiety at each of nucleotides at positions 2-7 and 12-36 in the sense strand is modified with a 2′-OMe. In some embodiments, for these oligonucleotides the sugar moiety at each of nucleotides at positions 1-6 and 12-36 in the sense strand is modified with a 2′-OMe. In some embodiments, for these oligonucleotides the sugar moiety at each of nucleotides at positions 1-7 and 12-15 and 17-36 in the sense strand is modified with a 2′-OMe. In some embodiments, for these oligonucleotides the sugar moiety at each of nucleotides at positions 1-7 and 12-19 and 21-36 in the sense strand is modified with a 2′-OMe. In some embodiments, for these oligonucleotides the sugar moiety at each of nucleotides at positions 1-7 and 12-22 and 24-36 in the sense strand is modified with a 2′-OMe. In some embodiments, for these oligonucleotides the sugar moiety at each of nucleotides at positions 1-7 and 12-27 and 29-36 in the sense strand is modified with a 2′-OMe. In some embodiments, for these oligonucleotides the sugar moiety at each of nucleotides at positions 1-7 and 12-28 and 30-36 in the sense strand is modified with a 2′-OMe. In some embodiments, for these oligonucleotides the sugar moiety at each of nucleotides at positions 1-7 and 12-29 and 31-36 in the sense strand is modified with a 2′-OMe.

In some embodiments, the sense strand comprises at least one 2′-F modified nucleotide wherein the remaining nucleotides not modified with a 2′-F group or conjugated to a lipid are modified with a 2′-OMe.

In some embodiments, the antisense strand has 7 nucleotides that are modified at the 2′ position of the sugar moiety with a 2′-F. In some embodiments, the sugar moiety at positions 2, 3, 4, 5, 7, 10, and 14 of the antisense strand are modified with a 2′-F. In some embodiments, the antisense strand has 14 nucleotides that are modified at the 2′ position of the sugar moiety with a 2′-OMe. In some embodiments, the sugar moiety at positions 6, 8, 9, 11, 12, 13, 15, 16, 17, 18, 19, 20, 21, and 22 of the antisense strand are modified with a 2′-OMe.

In some embodiments, the sense strand has 4 nucleotides that are modified at the 2′ position of the sugar moiety with a 2′-F. In some embodiments, the sugar moiety at positions 2, 3, 8, 9, 10, and 11 of the sense strand are modified with a 2′-F. In some embodiments, the sense strand has 15 nucleotides that are modified at the 2′ position of the sugar moiety with a 2′-OMe. In some embodiments, the sugar moiety at positions 6, 8, 9, 11, 12, 13, 15, 16, 17, 18, 19, 20, 21, and 22 of the antisense strand are modified with a 2′-OMe.

In some embodiments, the antisense strand has 3 nucleotides that are modified at the 2′-position of the sugar moiety with a 2′-F. In some embodiments, the sugar moiety at positions 2, 5 and 14 and optionally up to 3 of the nucleotides at positions 1, 3, 7 and 10 of the antisense strand are modified with a 2′-F. In some embodiments, the sugar moiety at each of the positions at positions 2, 5 and 14 of the antisense strand is modified with the 2′-F. In other embodiments, the sugar moiety at each of the positions at positions 1, 2, 5 and 14 of the antisense strand is modified with the 2′-F. In other embodiments, the sugar moiety at each of the positions at positions 2, 4, 5 and 14 of the antisense strand is modified with the 2′-F. In some embodiments, the sugar moiety at each of the positions at positions 1, 2, 3, 5, 7 and 14 of the antisense strand is modified with the 2′-F. In some embodiments, the sugar moiety at each of the positions at positions 2, 3, 4, 5, 7 and 14 of the antisense strand is modified with the 2′-F. In some embodiments, the sugar moiety at each of the positions at positions 1, 2, 3, 5, 10 and 14 of the antisense strand is modified with the 2′-F. In some embodiments, the sugar moiety at each of the positions at positions 2, 3, 4, 5, 10 and 14 of the antisense strand is modified with the 2′-F. In some embodiments, the sugar moiety at each of the positions at positions 2, 3, 5, 7, 10 and 14 of the antisense strand is modified with the 2′-F. In some embodiments, the sugar moiety at each of the positions at positions 2, 3, 4, 5, 7, 10 and 14 of the antisense strand is modified with the 2′-F. In some embodiments, the antisense strand has 9 nucleotides that are modified at the 2′-position of the sugar moiety with a 2′-F. In some embodiments, the sugar moiety at each of the positions at positions 2, 3, 4, 5, 7, 10, 14, 16 and 19 of the antisense strand is modified with the 2′-F.

In some embodiments, a lipid-conjugated RNAi oligonucleotide provided herein comprises an antisense strand having the sugar moiety of each of the nucleotides at positions 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 2, 3, 4, 5, 7, 10, 14, 16 and 19 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 1, 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 1, 2, 3, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 1, 2, 3, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an lipid-conjugated RNAi oligonucleotide provided herein comprises an antisense strand having the sugar moiety of each of the nucleotides at positions 2, 3, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 2, 3, 4, 5, 7, 10, 14, 16 and 19 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 2, 3, 4, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-F.

In some embodiments, a lipid-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-OMe.

In some embodiments, a lipid-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises a sense strand having the sugar moiety at positions 8-11 modified with 2′-F. In some embodiments, a lipid-conjugated RNAi oligonucleotide provided herein comprises a sense strand having the sugar moiety at positions 3, 5, 8, 10, 12, 13, 15 and 17 modified with 2′-F. In some embodiments, a lipid-conjugated RNAi oligonucleotide provided herein comprises a sense strand having the sugar moiety at positions 1-7 and 12-17 or 12-20 modified with 2′OMe. In some embodiments, a lipid-conjugated RNAi oligonucleotide provided herein comprises a sense strand having the sugar moiety at positions 2-7 and 12-17 or 12-20 modified with 2′OMe. In some embodiments, a lipid-conjugated RNAi oligonucleotide provided herein comprises a sense strand having the sugar moiety at positions 1-6 and 12-17 or 12-20 modified with 2′OMe. In some embodiments, a lipid-conjugated RNAi oligonucleotide provided herein comprises a sense strand having the sugar moiety at positions 1, 2, 4, 6, 7, 9, 11, 14, 16 and 18-20 modified with 2′OMe. In some embodiments, a lipid-conjugated RNAi oligonucleotide provided herein comprises a sense strand having the sugar moiety of each of the nucleotides at positions 1-7 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA). In some embodiments, a lipid-conjugated RNAi oligonucleotide provided herein comprises a sense strand having the sugar moiety of each of the nucleotides at positions 2-7 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA). In some embodiments, a lipid-conjugated RNAi oligonucleotide provided herein comprises a sense strand having the sugar moiety of each of the nucleotides at positions 1-6 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA). In some embodiments, a lipid-conjugated RNAi oligonucleotide provided herein comprises a sense strand having the sugar moiety at positions 1, 2, 4, 6, 7, 9, 11, 14, 16 and 18-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-F.

In some embodiments, a lipid-conjugated RNAi oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-OMe.

In some embodiments, a lipid-conjugated RNAi oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

### 5′-Terminal Phosphate

In some embodiments, a lipid-conjugated RNAi oligonucleotide described herein comprises a 5′-terminal phosphate. In some embodiments, the 5′-terminal phosphate groups of the lipid-conjugated RNAi oligonucleotide enhance the interaction with Ago2. However, oligonucleotides comprising a 5′-phosphate group may be susceptible to degradation via phosphatases or other enzymes, which can limit their bioavailability in vivo. In some embodiments, an lipid-conjugated RNAi oligonucleotide herein comprises analogs of 5′ phosphates that are resistant to such degradation. In some embodiments, the phosphate analog is oxymethyl phosphonate, vinyl phosphonate or malonyl phosphonate, or a combination thereof. In some embodiments, the 5′ end of a lipid-conjugated RNAi oligonucleotide strand is attached to chemical moiety that mimics the electrostatic and steric properties of a natural 5′-phosphate group (“phosphate mimic”).

In some embodiments, a lipid-conjugated RNAi oligonucleotide herein has a phosphate analog at a 4′-carbon position of the sugar (referred to as a “4′-phosphate analog”). See, e.g., Intl. Patent Application Publication No. WO 2018/045317. In some embodiments, a lipid-conjugated RNAi oligonucleotide herein comprises a 4′-phosphate analog at a 5′-terminal nucleotide. In some embodiments, a phosphate analog is an oxymethyl phosphonate, in which the oxygen atom of the oxymethyl group is bound to the sugar moiety (e.g., at its 4′-carbon) or analog thereof. In other embodiments, a 4′-phosphate analog is a thiomethyl phosphonate or an aminomethyl phosphonate, in which the sulfur atom of the thiomethyl group or the nitrogen atom of the amino methyl group is bound to the 4′-carbon of the sugar moiety or analog thereof. In some embodiments, a 4′-phosphate analog is an oxymethyl phosphonate. In some embodiments, an oxymethyl phosphonate is represented by the formula —O—CH₂—PO(OH)₂, —O—CH₂—PO(OR)₂, or —O—CH₂—POOH(R), in which R is independently selected from H, CH₃, an alkyl group, CH₂CH₂CN, CH₂OCOC(CH₃)₃, CH₂OCH₂CH₂Si(CH₃)₃ or a protecting group. In some embodiments, the alkyl group is CH₂CH₃. More typically, R is independently selected from H, CH₃ or CH₂CH₃. In some embodiment, R is CH₃. In some embodiments, the 4′-phosphate analog is 5′-methoxyphosphonate-4′-oxy. In some embodiments, the 4′-phosphate analog is 4′-oxymethylphosphonate.

In some embodiments, a lipid-conjugated RNAi oligonucleotide provided herein comprises an antisense strand comprising a 4′-phosphate analog at the 5′-terminal nucleotide, wherein 5′-terminal nucleotide comprises the following structure:

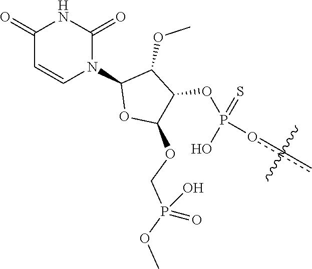

### 4′-O-monomethylphosphonate-2′-O-methyluridine phosphorothioate [MePhosphonate-40-mUs]

### Modified Internucleotide Linkage

In some embodiments, a lipid-conjugated RNAi oligonucleotide herein comprises a modified internucleotide linkage. In some embodiments, phosphate modifications or substitutions result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, a lipid-conjugated RNAi oligonucleotide provided herein has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 18 and 19 of the sense strand, positions 19 and 20 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand.

In some embodiments, an oligonucleotide conjugate described herein comprises a peptide nucleic acid (PNA). PNAs are oligonucleotide mimics in which the sugar-phosphate backbone has been replaced by a pseudopeptide skeleton, composed of N-(2-aminoethyl)glycine units. Nucleobases are linked to this skeleton through a two-atom carboxymethyl spacer. In some embodiments, an oligonucleotide conjugate described herein comprises a morpholino oligomer (PMO) comprising an internucleotide linkage backbone of methylene morpholine rings linked through phosphorodiamidate groups.

### Base Modifications

In some embodiments, a lipid-conjugated RNAi oligonucleotide herein comprises one or more modified nucleobases. In some embodiments, modified nucleobases (also referred to herein as base analogs) are linked at the 1′ position of a nucleotide sugar moiety. In some embodiments, a modified nucleobase is a nitrogenous base. In some embodiments, a modified nucleobase does not contain nitrogen atom. See, e.g., US Patent Application Publication No. 2008/0274462. In some embodiments, a modified nucleotide comprises a universal base. In some embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. In some embodiments, when compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, US Patent Application Publication No. 2007/0254362; Van Aerschot et al. (1995) NUCLEIC ACIDS RES. 23:4363-4370; Loakes et al. (1995) NUCLEIC ACIDS RES. 23:2361-66; and Loakes & Brown (1994) NUCLEIC ACIDS RES. 22:4039-43).

### Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance. See US Patent Application Publication No. 2011/0294869, Intl. Patent Application Publication Nos. WO 2014/088920 and WO 2015/188197, and Meade et al., (2014) NAT. BIOTECHNOL. 32:1256-63. This reversible modification of the internucleotide diphosphate linkages is designed to be cleaved intracellularly by the reducing environment of the cytosol (e.g., glutathione). Earlier examples include neutralizing phosphotriester modifications that were reported to be cleavable inside cells (see, Dellinger et al., (2003) J. AM. CHEM. SOC. 125:940-50).

In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed, and the result is a cleaved oligonucleotide. Using reversible, glutathione-sensitive moieties, it is possible to introduce sterically larger chemical groups into the oligonucleotide of interest when compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′-carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. In some embodiments, the glutathione-sensitive moiety is located at the 3′-carbon of sugar, particularly when the modified nucleotide is the 3′-terminal nucleotide of the oligonucleotide. In some embodiments, the glutathione-sensitive moiety comprises a sulfonyl group. See, e.g., U.S. Provisional Patent Application No. 62/378,635, entitled Compositions Comprising Reversibly Modified Oligonucleotides and Uses Thereof, which was filed on Aug. 23, 2016.

### Targeting Ligands

In some embodiments, it is desirable to target the oligonucleotides of the disclosure (e.g., lipid-conjugated RNAi oligonucleotides) to one or more cells or tissues of the central nervous system (CNS). Such a strategy can help to avoid undesirable effects in other organs or avoid undue loss of the oligonucleotide to cells, tissue or organs that would not benefit from the oligonucleotide. Accordingly, in some embodiments, a lipid-conjugated RNAi oligonucleotide disclosed herein is modified to facilitate targeting and/or delivery to a particular tissue, cell, or organ (e.g., to facilitate delivery of the conjugate to the CNS). In some embodiments, a lipid-conjugated RNAi oligonucleotide comprises at least one nucleotide (e.g., 1, 2, 3, 4, 5, 6 or more nucleotides) conjugated to one or more targeting ligand(s).

In some embodiments, 1 or more (e.g., 1, 2, 3, 4, 5 or 6) nucleotides of a lipid-conjugated RNAi oligonucleotide disclosed herein are each conjugated to a separate targeting ligand. In some embodiments, 1 nucleotide of a lipid-conjugated RNAi oligonucleotide herein is conjugated to a separate targeting ligand. In some embodiments, 2 to 4 nucleotides of a lipid-conjugated RNAi oligonucleotide herein are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ end of the sense or antisense strand) such that the targeting ligands resemble bristles of a toothbrush and the lipid-conjugated RNAi oligonucleotide resembles a toothbrush. For example, a lipid-conjugated RNAi oligonucleotide may comprise a stem-loop at either the 5′ or 3′ end of the sense strand and 1, 2, 3 or 4 nucleotides of the loop of the stem may be individually conjugated to a targeting ligand. In some embodiments, a lipid-conjugated RNAi oligonucleotide provided by the disclosure comprises a stem-loop at the 3′ end of the sense strand, wherein the loop of the stem-loop comprises a triloop or a tetraloop, and wherein the 3 or 4 nucleotides comprising the triloop or tetraloop, respectfully, are individually conjugated to a targeting ligand.

GalNAc is a high affinity ligand for the ASGPR, which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotide of the instant disclosure can be used to target these oligonucleotides to the ASGPR expressed on cells. In some embodiments, an oligonucleotide of the instant disclosure is conjugated to at least one or more GalNAc moieties, wherein the GalNAc moieties target the oligonucleotide to an ASGPR expressed on human liver cells (e.g., human hepatocytes). In some embodiments, the GalNAc moiety target the oligonucleotide to the liver.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3 or 4 monovalent GalNAc moieties, and is typically conjugated to 3 or 4 monovalent GalNAc moieties). In some embodiments, an oligonucleotide is conjugated to one or more bivalent GalNAc, trivalent GalNAc or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of a tetraloop are each conjugated to a separate GalNAc. In some embodiments, 1 to 3 nucleotides of a triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, four (4) GalNAc moieties can be conjugated to nucleotides in the tetraloop of the sense strand where each GalNAc moiety is conjugated to 1 nucleotide.

In some embodiments, the tetraloop is any combination of adenine and guanine nucleotides.

In some embodiments, the tetraloop (L) has a monovalent GalNAc moiety attached to any one or more guanine nucleotides of the tetraloop via any linker described herein, as depicted below (X=heteroatom):

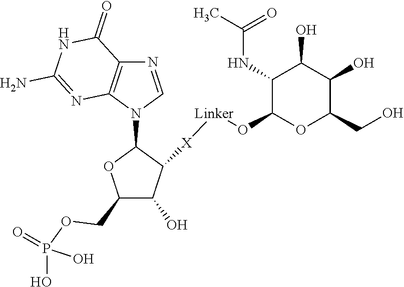

In some embodiments, the tetraloop (L) has a monovalent GalNAc attached to any one or more adenine nucleotides of the tetraloop via any linker described herein, as depicted below (X=heteroatom):

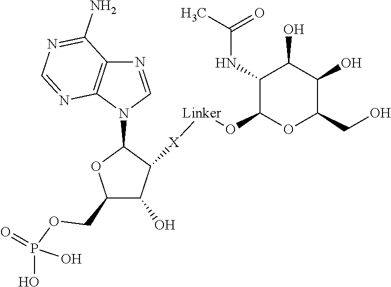

In some embodiments, a lipid-conjugated RNAi oligonucleotide herein comprises a monovalent GalNAc attached to a guanine nucleotide referred to as [ademG-GalNAc] or 2′-aminodiethoxymethanol-Guanine-GalNAc, as depicted below:

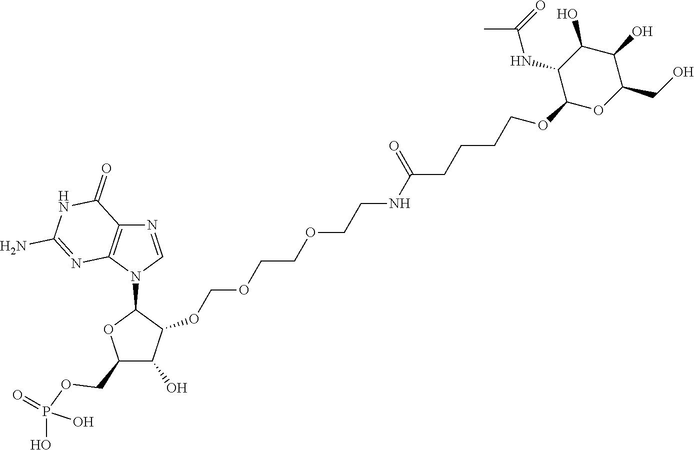 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, a lipid-conjugated RNAi oligonucleotide herein comprises a monovalent GalNAc attached to an adenine nucleotide, referred to as [ademA-GalNAc] or 2′-aminodiethoxymethanol-Adenine-GalNAc, as depicted below:

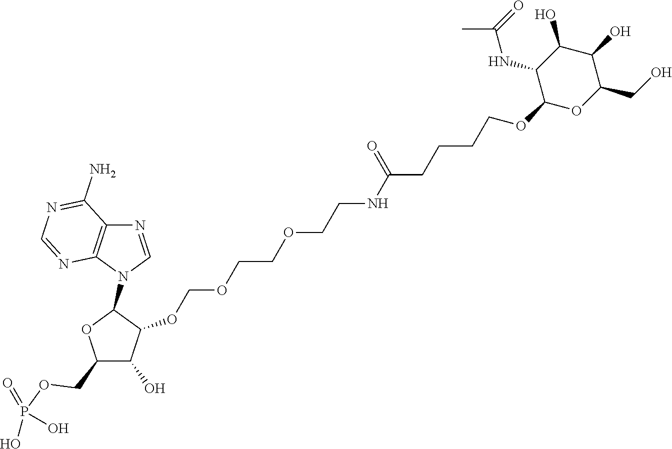 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

An example of such conjugation is shown below for a loop comprising from 5′ to 3′ the nucleotide sequence GAAA (L=linker, X=heteroatom). Such a loop may be present, for example, at positions 27-30 of the sense strand. In the chemical formula, [symbol] is used to describe an attachment point to the oligonucleotide strand.

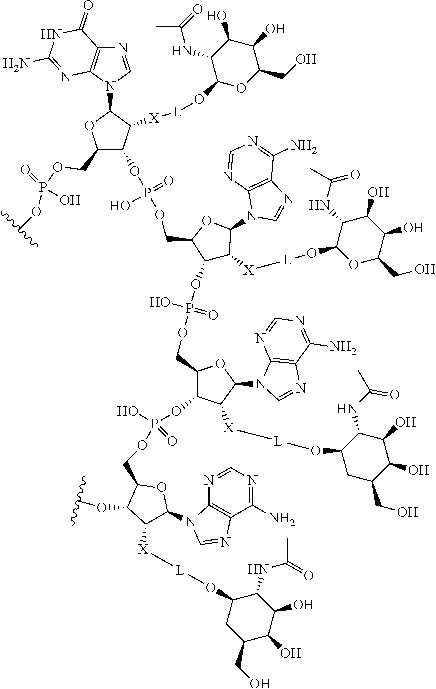

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is stable. Examples are shown below for a loop comprising from 5′ to 3′ the nucleotides GAAA, in which GalNAc moieties are attached to nucleotides of the loop using an acetal linker. Such a loop may be present, for example, at positions 27-30 of the sense strand. In the chemical formula, [symbol] is an attachment point to the oligonucleotide strand.

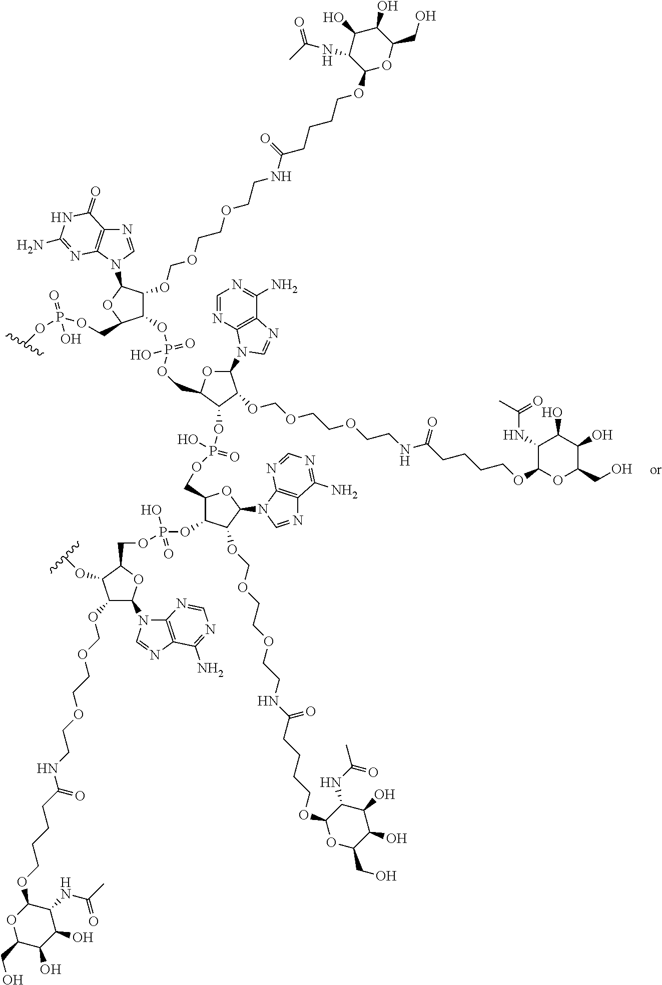 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

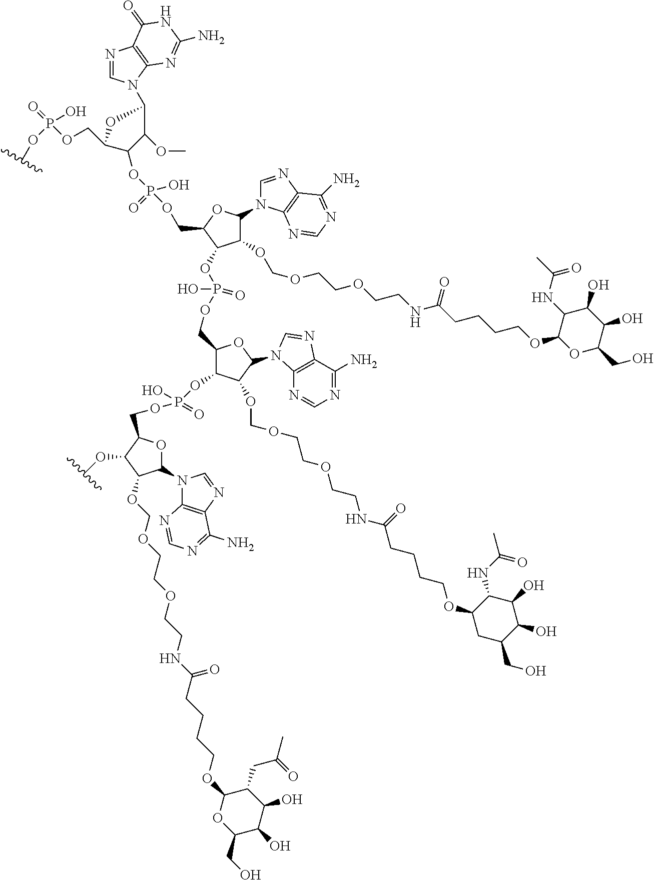

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and a lipid-conjugated RNAi oligonucleotide. In some embodiments, a lipid-conjugated RNAi oligonucleotide herein does not have a GalNAc conjugated thereto.

### Lipid Conjugates

In some embodiments, any of the lipid moieties described herein are conjugated to a nucleotide of the sense strand of the oligonucleotide. In some embodiments, a lipid moiety is conjugated to a terminal position of the oligonucleotide. In some embodiments, the lipid moiety is conjugated to the 5′ terminal nucleotide of the sense strand. In some embodiments, the lipid moiety is conjugated to the 3′ terminal nucleotide of the sense strand.

In some embodiments, the lipid moiety is conjugated to an internal nucleotide on the sense strand. An internal position is any nucleotide position other than the two terminal positions from each end of the sense strand. In some embodiments, the lipid moiety is conjugated to one or more internal positions of the sense strand. In some embodiments, the lipid moiety is conjugated to position 1, position 2, position 3, position 4, position 5, position 6, position 7, position 8, position 9, position 10, position 11, position 12, position 13, position 14, position 15, position 16, position 17, position 18, position 19, or position 20 of a sense strand. In some embodiments, the lipid moiety is conjugated to position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 of a sense strand. In some embodiments, the lipid moiety is conjugated to position 1 of the sense strand. In some embodiments, the lipid moiety is conjugated to position 7 of the sense strand. In some embodiments, the lipid moiety is conjugated to position 16 of the sense strand. In some embodiments, the lipid moiety is conjugated to position 20 of the sense strand. In some embodiments, the lipid moiety is conjugated to position 23 of the sense strand. In some embodiments, the lipid moiety is conjugated to position 28 of the sense strand. In some embodiments, the lipid moiety is conjugated to position 29 of the sense strand. In some embodiments, the lipid moiety is conjugated to position 30 of the sense strand.

In some embodiments a lipid-conjugated RNAi oligonucleotide described herein comprises at least one nucleotide conjugated with one or more lipid moieties. In some embodiments, the one or more lipid moieties are conjugated to the same nucleotide. In some embodiments, the one or more lipid moieties are conjugated to different nucleotides. In some embodiments, one, two, three, four, five, or six lipid moieties are conjugated to the oligonucleotide.

In some embodiments, the lipid moiety is a hydrocarbon chain. In some embodiments, the hydrocarbon chain is saturated. In some embodiments, the hydrocarbon chain is unsaturated. In some embodiments, the hydrocarbon chain is branched. In some embodiments, the hydrocarbon chain is straight. In some embodiments, the lipid moiety is a C8-C30 hydrocarbon chain. In some embodiments, the lipid moiety is a C8:0, C10:0, C11:0, C12:0, C14:0, C16:0, C17:0, C18:0, C18:1, C18:2, C22:5, C22:0, C24:0, C26:0, C22:6, C24:1, diacyl C16:0 or diacyl C18:1.

In some embodiments, the lipid moiety is a C16 hydrocarbon chain.

In some embodiments, a lipid-conjugated RNAi oligonucleotide described herein comprises a nucleotide sequence and one or more targeting ligands, wherein the nucleotide sequence comprises one or more nucleosides (nucleic acids) conjugated with one or more targeting ligands represented by formula II-a:

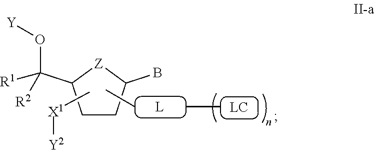

or a pharmaceutically acceptable salt thereof,
wherein:
B is a nucleobase or hydrogen;
R¹ and R² are independently hydrogen, halogen, R^{A}, —CN, —S(O)R, —S(O)₂R, —Si(OR)₂R, —Si(OR)R₂, or —SiR₃; or
   R¹ and R² on the same carbon are taken together with their intervening atoms to form a 3-7 membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur; or
two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
each LC is a lipid conjugate moiety; and wherein each LC is independently a lipid conjugate moiety comprising a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, —V¹CR²W¹—, or

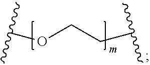

m is 1-50;
X¹, V¹ and W¹ are independently —C(R)₂—, —OR, —O—, —S—, —Se—, or —NR—;
Y is hydrogen, a suitable hydroxyl protecting group,

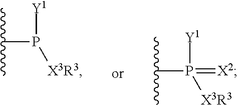

R³ is hydrogen, a suitable protecting group, a suitable prodrug, or an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X² is O, S, or NR;
X³ is —O—, —S—, —BH₂—, or a covalent bond;
Y¹ is a linking group attaching to the 2′- or 3′-terminal of a nucleoside, a nucleotide, or an oligonucleotide;
Y² is hydrogen, a suitable protecting group, a phosphoramidite analogue, an internucleotide linking group attaching to the 5′-terminal of a nucleoside, a nucleotide, or an oligonucleotide, or a linking group attaching to a solid support; and
Z is —O—, —S—, —NR—, or —CR₂—.

In some embodiments, the lipid moiety is conjugated to the oligonucleotide via a linker. In some embodiments, a nucleotide of the lipid-conjugated oligonucleotide is represented by formula II-b or II-c:

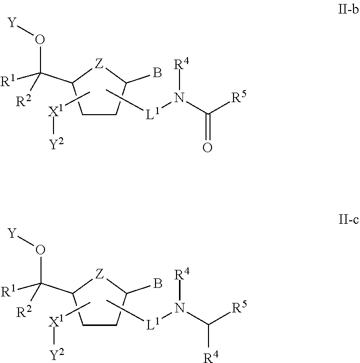

or a pharmaceutically acceptable salt thereof, wherein:
L¹ is a covalent bond, a monovalent or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or

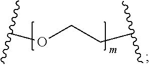

R⁴ is hydrogen, R^{A}, or a suitable amine protection group; and
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR.

In some embodiments of the lipid-conjugated RNAi oligonucleotide, R⁵ is selected from

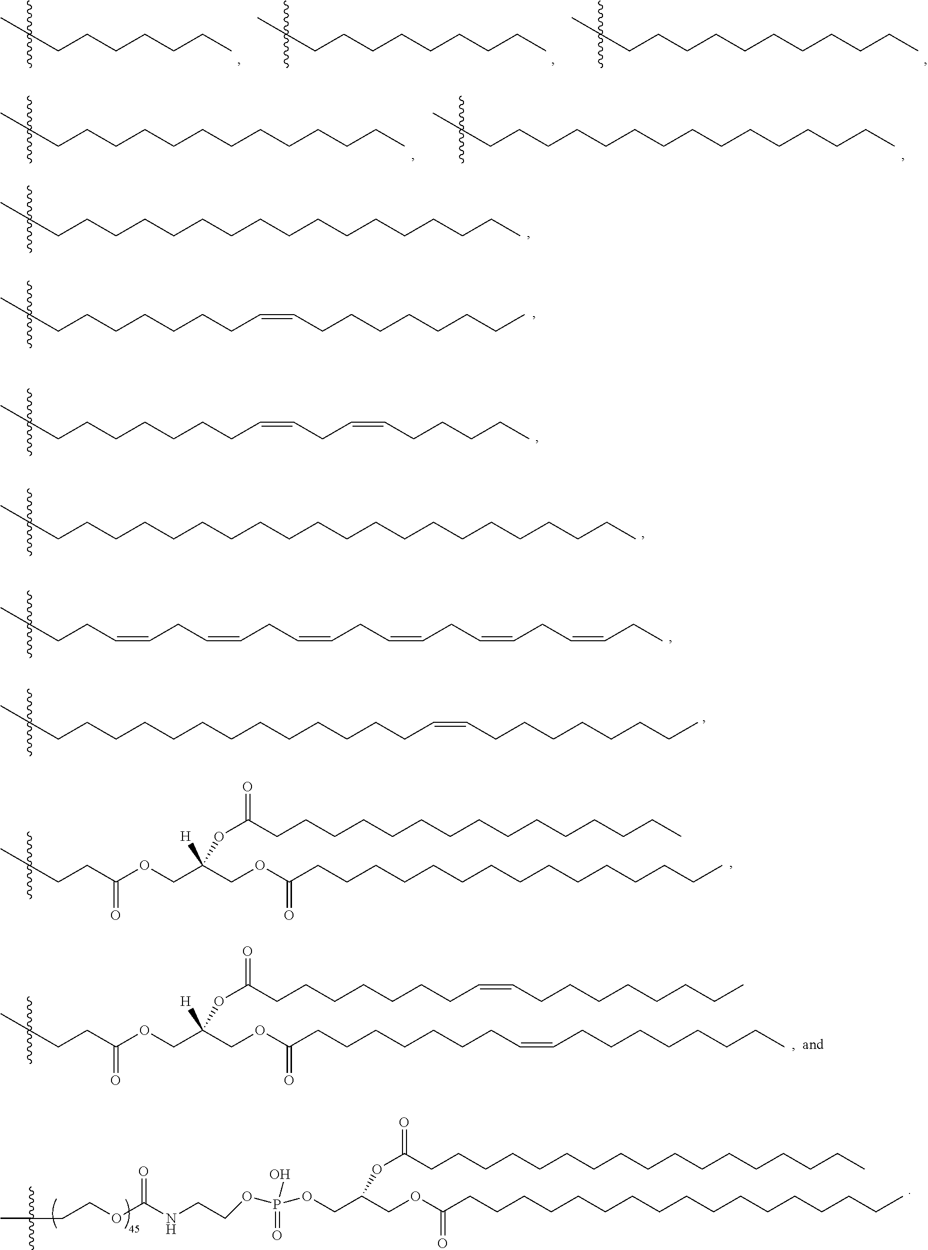

In certain embodiments of the lipid-conjugated RNAi oligonucleotide, R⁵ is selected from

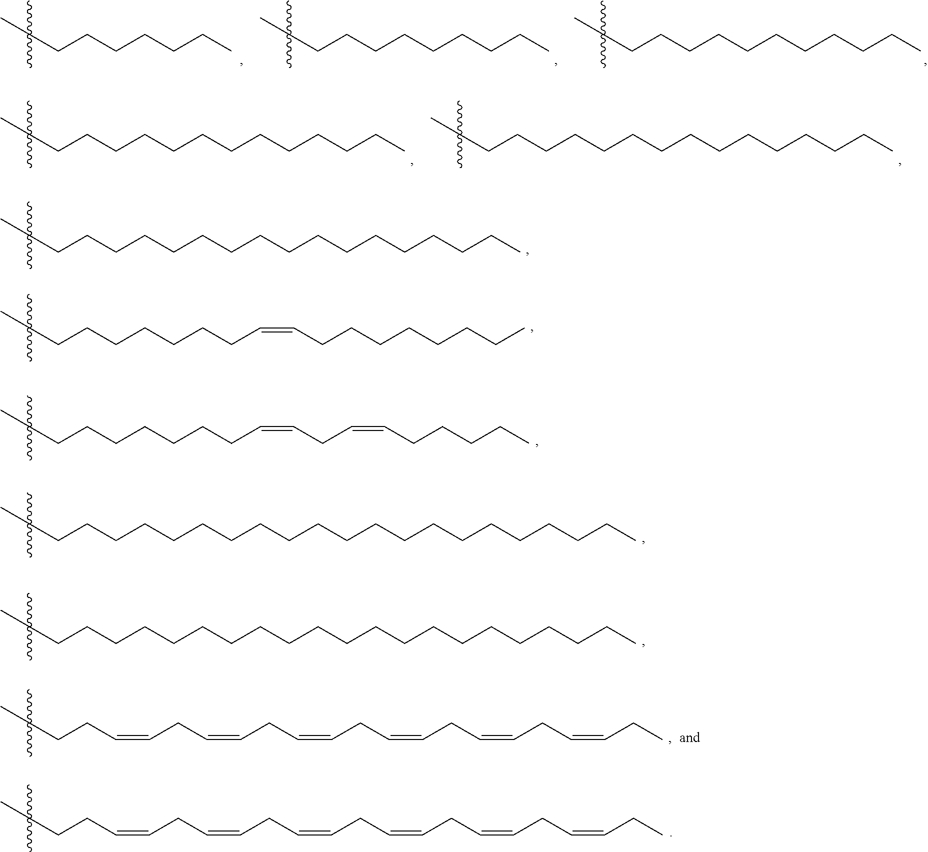

In some embodiments, R⁵ is

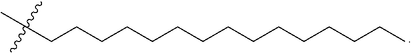 [SMILES: *CCCCCCCCCCCCCCC]

In some embodiments, R⁵ is

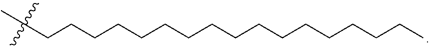 [SMILES: *CCCCCCCCCCCCCCCCC]

In some embodiments, a nucleotide of the lipid-conjugated RNAi oligonucleotide is represented by formula II-Ib or II-Ic:

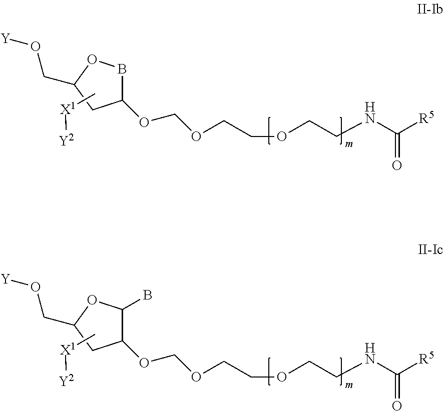

or a pharmaceutically acceptable salt thereof; wherein
B is a nucleobase or hydrogen;
m is 1-50;
X¹ is —O—, or —S—;

Y is hydrogen,

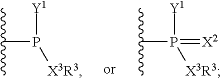

X³R³, or X³R³;
R³ is hydrogen, or a suitable protecting group;
X² is O, or S;
X³ is —O—, —S—, or a covalent bond;
Y¹ is a linking group attaching to the 2′- or 3′-terminal of a nucleoside, a nucleotide, or an oligonucleotide;
Y² is hydrogen, a phosphoramidite analogue, an internucleotide linking group attaching to the 5′-terminal of a nucleoside, a nucleotide, or an oligonucleotide, or a linking group attaching to a solid support;
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—; and
R is hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, the lipid is selected from

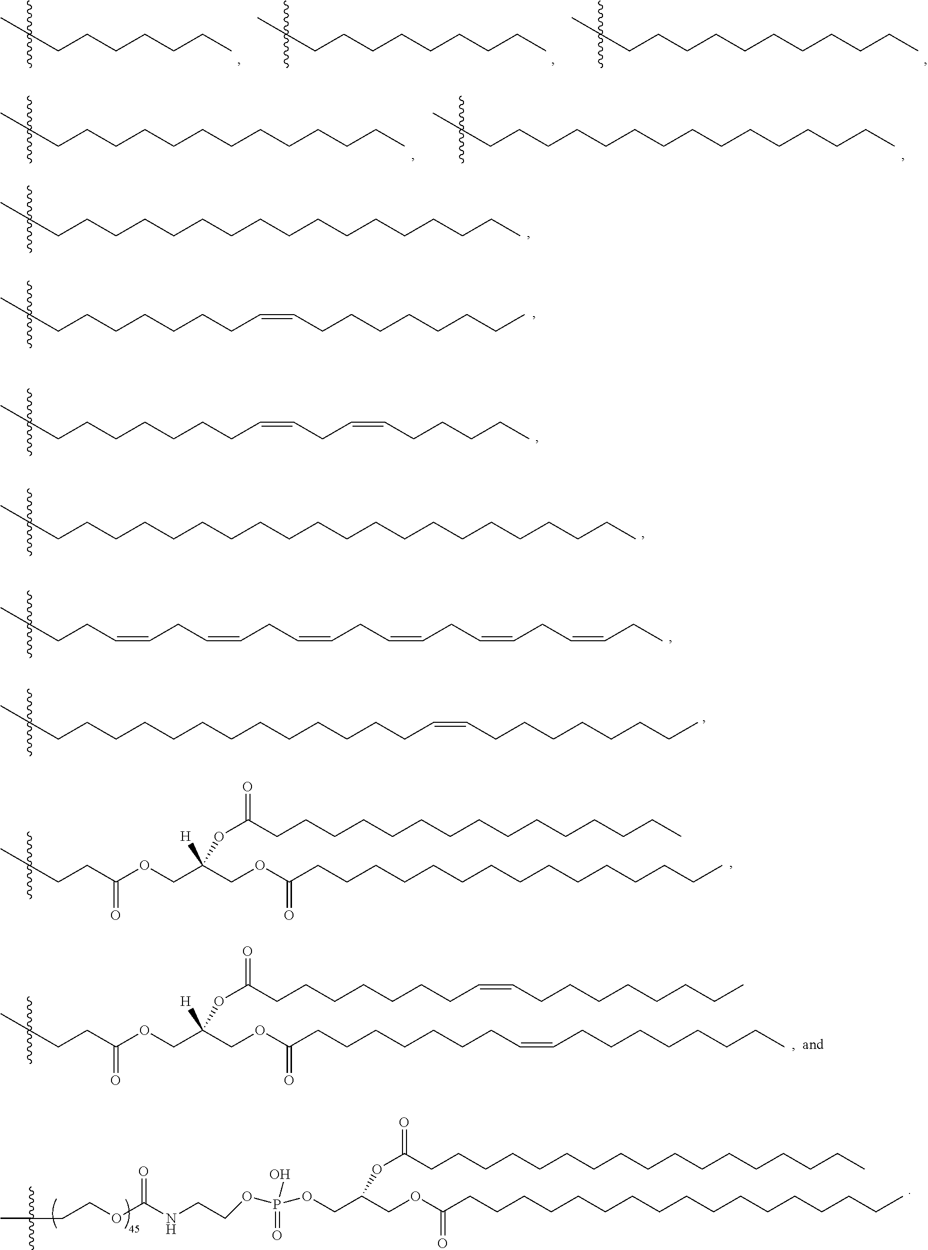

In some embodiments, R⁵ is

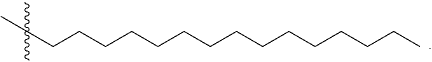 [SMILES: *CCCCCCCCCCCCCCC]

In some embodiments, the oligonucleotide of the oligonucleotide-ligand conjugate is a double-stranded molecule. In some embodiments, the oligonucleotide is an RNAi molecule. In some embodiments, the double stranded oligonucleotide comprises a stem loop. In some embodiments, the stem loop is set forth as S1-L-S2, wherein S1 is complementary to S2, and wherein L forms a loop between S1 and S2. In some embodiments, the ligand is conjugated to any of the nucleotides in the loop of the stem loop. In some embodiments, the ligand is conjugated to any of the nucleotides in the stem of the stem loop. In some embodiments, the ligand is conjugated to the first nucleotide from 5′ to 3′ in the loop. In some embodiments, the ligand is conjugated to the second nucleotide from 5′ to 3′ in the loop. In some embodiments, the ligand is conjugated to the third nucleotide from 5′ to 3′ in the loop. In some embodiments, the ligand is conjugated to the fourth nucleotide from 5′ to 3′ in the loop. In some embodiments, the ligand is conjugated to one, two, three, or four of the nucleotides in the loop. In some embodiments, the ligand is conjugated to three of the nucleotides in the stem loop.

In some embodiments, the stem loop is 16 nucleotides in length. In some embodiments, the ligand is conjugated to the third nucleotide from 5′ to 3′ in the stem loop. In some embodiments, the ligand is conjugated to the eighth nucleotide from 5′ to 3′ in the stem loop. In some embodiments, the ligand is conjugated to the ninth nucleotide from 5′ to 3′ in the stem loop. In some embodiments, the ligand is conjugated to the tenth nucleotide from 5′ to 3′ in the stem loop. In some embodiments, the ligand is conjugated to one, two, three, or four of the nucleotides in the stem loop. In some embodiments, the ligand is conjugated to three of the nucleotides in the stem loop.

In some embodiments, the lipid-conjugated RNAi oligonucleotide comprises a sense strand of 20 nucleotides with positions numbered 1-20 from 5′ to 3′. In some embodiments, the lipid-conjugated RNAi oligonucleotide comprises a lipid conjugated to position 1 of a 20-nucleotide sense strand. In some embodiments, the lipid-conjugated RNAi oligonucleotide comprises a lipid conjugated to position 7 of a 20-nucleotide sense strand. In some embodiments, the lipid-conjugated RNAi oligonucleotide comprises a sense strand of 36 nucleotides with positions numbered 1-36 from 5′ to 3′. In some embodiments, the lipid-conjugated RNAi oligonucleotide- comprises a lipid conjugated to position 1 of a 36-nucleotide sense strand. In some embodiments, the lipid-conjugated RNAi oligonucleotide- comprises a lipid conjugated to position 7 of a 36-nucleotide sense strand. In some embodiments, the lipid-conjugated RNAi oligonucleotide- comprises a lipid conjugated to position 16 of a 36-nucleotide sense strand. In some embodiments, the lipid-conjugated RNAi oligonucleotide-comprises a lipid conjugated to position 20 of a 36-nucleotide sense strand. In some embodiments, the lipid-conjugated RNAi oligonucleotide- comprises a lipid conjugated to position 23 of a 36-nucleotide sense strand. In some embodiments, the lipid-conjugated RNAi oligonucleotide- comprises a lipid conjugated to position 28 of a 36-nucleotide sense strand.

In some embodiments, the lipid-conjugated RNAi oligonucleotide- comprises a lipid conjugated to position 29 of a 36-nucleotide sense strand. In some embodiments, the lipid-conjugated RNAi oligonucleotide- comprises a lipid conjugated to position 30 of a 36-nucleotide sense strand.

### Exemplary Oligonucleotides

In some embodiments, the lipid-conjugated RNAi oligonucleotide comprises a nucleotide conjugated with a fatty acid. In some embodiments, the fatty acid is a saturated fatty acid. In some embodiments, the fatty acid is an unsaturated fatty acid. In some embodiments, lipid-conjugated RNAi oligonucleotide comprises a nucleotide conjugated with a lipid. In some embodiments, the lipid is a carbon chain. In some embodiments, the carbon chain is saturated. In some embodiments, the carbon chain is unsaturated. In some embodiments, the lipid-conjugated RNAi oligonucleotide comprises a nucleotide conjugated with a 16-carbon (C16) lipid. In some embodiments, the C16 lipid comprises at least one double bond.

In some embodiments, the oligonucleotide of the lipid-conjugated RNAi oligonucleotide is conjugated to a C16 lipid as shown in:

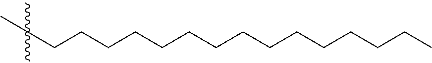 [SMILES: *CCCCCCCCCCCCCCC]

In some embodiments, the lipid-conjugated RNAi oligonucleotide comprises a sense strand of 20 nucleotides in length. In some embodiments, the lipid-conjugated RNAi oligonucleotide comprises an anti-sense strand of 22 nucleotides in length. In some embodiments, the sense strand is 20 nucleotides in length and the antisense strand in 22 nucleotides in length. In some embodiments, lipid-conjugated RNAi oligonucleotide comprises a sense strand of 20 nucleotides in length and an antisense strand in 22 nucleotides in length, wherein the sense and antisense strands form a duplex region of 20 base pairs.

In some embodiments, the 3′ end of the sense strand is a blunt end. In some embodiments, the 5′ end of the antisense strand is a blunt end. In some embodiments, the 3′ end of the antisense strand comprises an overhang. In some embodiments, the overhang is 2 nucleotides in length. In some embodiments the overhang is GG.

In some embodiments, the lipid-conjugated RNAi oligonucleotide comprises one or more 2′ modifications. In some embodiments, the 2′ modifications are selected from 2′-fluoro and 2′-methyl.

In some embodiments, a lipid-conjugated RNAi oligonucleotide comprises an antisense strand and a sense strand described herein, wherein the sense strand comprises at least one hydrocarbon chain conjugated to the 5′ terminal nucleotide of the sense strand. In some embodiments, a lipid-conjugated RNAi oligonucleotide comprises an antisense strand and a sense strand described herein, wherein the sense strand comprises at least one C16 hydrocarbon chain conjugated to the 5′ terminal nucleotide of the sense strand.

In some embodiments, a lipid-conjugated RNAi oligonucleotide comprises an antisense strand of 22-24 nucleotides and a sense strand of 20-22 nucleotides described herein, wherein the antisense and sense strands form a duplex region of 20-22 base pairs, wherein the sense strand comprises at least one hydrocarbon chain conjugated to the 5′ terminal nucleotide of the sense strand. In some embodiments, a lipid-conjugated RNAi oligonucleotide comprises an antisense strand of 22-24 nucleotides and a sense strand of 20-22 nucleotides described herein, wherein the antisense and sense strands form a duplex region of 20-22 base pairs, wherein the sense strand comprises at least one C16 hydrocarbon chain conjugated to the 5′ terminal nucleotide of the sense strand.

In some embodiments, a lipid-conjugated RNAi oligonucleotide comprises an antisense strand of 22-24 nucleotides and a sense strand of 20-22 nucleotides described herein, wherein the antisense and sense strands form an asymmetric duplex region of 20-22 base pairs having an overhang on the 3′ end of the antisense strand and a blunt-end at the 3′ end of the oligonucleotide, wherein the sense strand comprises at least one hydrocarbon chain conjugated to the 5′ terminal nucleotide of the sense strand. In some embodiments, a lipid-conjugated RNAi oligonucleotide comprises an antisense strand of 22-24 nucleotides and a sense strand of 20-22 nucleotides described herein, wherein the antisense and sense strands form an asymmetric duplex region of 20-22 base pairs having an overhang on the 3′ end of the antisense strand and a blunt-end at the 3′ end of the oligonucleotide, wherein the sense strand comprises at least one C16 hydrocarbon chain conjugated to the 5′ terminal nucleotide of the sense strand.

In some embodiments, a lipid-conjugated RNAi oligonucleotide comprises an antisense strand and a sense strand described herein, wherein the sense strand comprises at least one hydrocarbon chain conjugated to an internal nucleotide of the sense strand (e.g., nucleotide at position 7). In some embodiments, a lipid-conjugated RNAi oligonucleotide comprises an antisense strand and a sense strand described herein, wherein the sense strand comprises at least one C16 hydrocarbon chain conjugated to an internal nucleotide of the sense strand (e.g., nucleotide at position 7). In some embodiments, not all internal nucleotides are suitable for lipid conjugation for delivery of an RNAi oligonucleotide to a neuron of the CNS. For example, in some embodiments, conjugation at positions 9 or 10 of a sense strand numbered from 5′ to 3′ is not suitable for delivery of an RNAi oligonucleotide to a neuron of the CNS. In some embodiments, lipid conjugation at an internal position of a sense strand numbered from 5′ to 3′ excludes positions 9 and 10.

In some embodiments, a lipid-conjugated RNAi oligonucleotide comprises an antisense strand of 22-24 nucleotides and a sense strand of 20-22 nucleotides described herein, wherein the antisense and sense strands form a duplex region of 20-22 base pairs, wherein the sense strand comprises at least one hydrocarbon chain conjugated to an internal nucleotide of the sense strand (e.g., nucleotide at position 7). In some embodiments, a lipid-conjugated RNAi oligonucleotide comprises an antisense strand of 22-24 nucleotides and a sense strand of 20-22 nucleotides described herein, wherein the antisense and sense strands form a duplex region of 20-22 base pairs, wherein the sense strand comprises at least one C16 hydrocarbon chain conjugated to an internal nucleotide of the sense strand (e.g., nucleotide at position 7).

In some embodiments, a lipid-conjugated RNAi oligonucleotide comprises an antisense strand of 22-24 nucleotides and a sense strand of 20-22 nucleotides described herein, wherein the antisense and sense strands form an asymmetric duplex region of 20-22 base pairs having an overhang on the 3′ end of the antisense strand and a blunt-end at the 3′ end of the oligonucleotide, wherein the sense strand comprises at least one hydrocarbon chain conjugated to an internal nucleotide of the sense strand (e.g., nucleotide at position 7). In some embodiments, a lipid-conjugated RNAi oligonucleotide comprises an antisense strand of 22-24 nucleotides and a sense strand of 20-22 nucleotides described herein, wherein the antisense and sense strands form an asymmetric duplex region of 20-22 base pairs having an overhang on the 3′ end of the antisense strand and a blunt-end at the 3′ end of the oligonucleotide, wherein the sense strand comprises at least one C16 hydrocarbon chain conjugated to an internal nucleotide of the sense strand (e.g., nucleotide at position 7).

In some embodiments, a lipid-conjugated RNAi oligonucleotide for reducing expression of a neuronal target gene comprises the modification pattern of

Sense Strand:
5′-[ademX-Ls][mX][mX][mX][mX][mX][mX][fX][fX][fX]
[fX][mX][mX][mX][mX][mX][mX][mXs][mXs][mX]-3′
Hybridized to:
Antisense Strand:
5′-[MePhosphonate-4O-mXs][fXs][fXs][fX][fX][mX][fX]
[mX][mX][fX][mX][mX][mX][fX][mX][mX][mX]
[mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-Ls]=Lipid attached to a nucleotide with a phosphorothioate linkage to the neighboring nucleotide, and [ademX-L]=Lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][ademX-L]
[fX][fX][fX][fX][mX][mX][mX][mX][mX][mX][mXs]
[mXs][mX]-3′
Hybridized to:
Antisense Strand:
5′-[MePhosphonate-4O-mXs][fXs][fXs][fX][fX][mX][fX]
[mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-L]=Lipid attached to a nucleotide.

In some embodiments, a lipid-conjugated RNAi oligonucleotide for reducing expression of a neuronal target gene comprises the modification pattern of

Sense Strand:
5′ [mXs][mX][mX][mX][mX][mX][mX][fX][fX][fX][fX]
[mX][mX][mX][mX][mX][mX][mXs][mXs][ademX-L]-3′
Hybridized to:
Antisense Strand:
5′-[MePhosphonate-4O-mXs][fXs][fXs][fX][f][mX][fX]
[mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-L]=Lipid attached to a nucleotide.

In some embodiments, a lipid-conjugated RNAi oligonucleotide for reducing expression of a neuronal target gene comprises the modification pattern of

Sense Strand:
5′-[ademX-C16s][mX][mX][mX][mX][mX][mX][fX][fX][fX]
[fX][mX][mX][mX][mX][mX][mX][mXs][mXs][mX]-3′
Hybridized to:
Antisense Strand:
5′-[MePhosphonate-4O-mXs][fXs][fXs][fX][fX][mX][fX]
[mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-C16s]=C16 lipid attached to a nucleotide with a phosphorothioate linkage to the neighboring nucleotide, and [ademX-C16]=C16 lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][ademX-C16][fX][fX][fX]
[fX][mX][mX][mX][mX][mX][mX][mXs][mXs][mX]-3′
Hybridized to:
Antisense Strand:
5′-[MePhosphonate-4O-mXs][fXs][fXs][fX][fX][mX]
[fX][mX]
[mX][fX][mX][mX][mX][fX][mX][mX][mX][mX][mX]
[mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-C16]=C16 lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][mX]
[fX][fX][fX][fX][mX]
[mX][mX][mX][mX][mX][mXs][mXs][ademX-C16]-3′
Hybridized to:
Antisense Strand:
5′-[MePhosphonate-4O-mXs][fXs][fXs][fX][fX]
[mX][fX][mX][mX][fX][mX][mX][mX][fX][mX][mX]
[mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-C16]=C16 lipid attached to a nucleotide.

In some embodiments, the lipid-conjugated RNAi oligonucleotide comprises a sense strand of 36 nucleotides in length. In some embodiments, the lipid-conjugated RNAi oligonucleotide comprises an anti-sense strand of 22 nucleotides in length. In some embodiments, the sense strand is 36 nucleotides in length and the antisense strand in 22 nucleotides in length. In some embodiments, a lipid-conjugated RNAi oligonucleotide comprises a sense strand of 36 nucleotides in length and an antisense strand in 22 nucleotides in length, wherein the sense and antisense strands form a duplex region of 20 base pairs.

In some embodiments, the 3′ end of the sense strand comprises a stem-loop. In some embodiments, the 3′ end of the sense strand comprises a tetraloop. In some embodiments, the 3′ end of the sense strand comprises a stem-loop comprising the sequence of SEQ ID NO: 21. In some embodiments, the 3′ end of the antisense strand comprises an overhang. In some embodiments, the overhang is 2 nucleotides in length. In some embodiments the overhang is GG.

In some embodiments, a lipid-conjugated RNAi oligonucleotide comprising a sense strand comprising a stem-loop at its 3′ end and at least one hydrocarbon chain conjugated to a nucleotide of the sense strand, reduces expression of a neuronal mRNA in the spinal cord. In some embodiments, a lipid-conjugated RNAi oligonucleotide comprising a sense strand comprising a stem-loop at its 3′ end and at least one hydrocarbon chain conjugated to a nucleotide of the sense strand, reduces expression of a neuronal mRNA in the lumbar spinal cord. In some embodiments, a lipid-conjugated RNAi oligonucleotide comprising a sense strand comprising a stem-loop at its 3′ end and at least one hydrocarbon chain conjugated to a nucleotide of the sense strand, reduces expression of a neuronal mRNA in the thoracic spinal cord. In some embodiments, a lipid-conjugated RNAi oligonucleotide comprising a sense strand comprising a stem-loop at its 3′ end and at least one hydrocarbon chain conjugated to a nucleotide of the sense strand, reduces expression of a neuronal mRNA in the cervical spinal cord.

In some embodiments, a lipid-conjugated RNAi oligonucleotide for reducing expression of a neuronal mRNA in the spinal cord comprises a sense strand comprising a stem-loop at its 3′ end and at least one hydrocarbon chain conjugated to a nucleotide of the sense strand. In some embodiments, a lipid-conjugated RNAi oligonucleotide for reducing expression of a neuronal mRNA in the lumbar spinal cord comprises a sense strand comprising a stem-loop at its 3′ end and at least one hydrocarbon chain conjugated to a nucleotide of the sense strand. In some embodiments, a lipid-conjugated RNAi oligonucleotide for reducing expression of a neuronal mRNA in the thoracic spinal cord comprises a sense strand comprising a stem-loop at its 3′ end and at least one hydrocarbon chain conjugated to a nucleotide of the sense strand. In some embodiments, a lipid-conjugated RNAi oligonucleotide for reducing expression of a neuronal mRNA in the cervical spinal cord comprises a sense strand comprising a stem-loop at its 3′ end and at least one hydrocarbon chain conjugated to a nucleotide of the sense strand.

In some embodiments, a lipid-conjugated RNAi oligonucleotide comprises a sense strand comprising a stem-loop at its 3′ end and at least one hydrocarbon chain conjugated to the 5′ terminal nucleotide of the sense strand. In some embodiments, a lipid-conjugated RNAi oligonucleotide comprises a sense strand comprising a stem-loop at its 3′ end and at least one hydrocarbon chain conjugated to a nucleotide of the sense strand. In some embodiments, a lipid-conjugated RNAi oligonucleotide comprises a sense strand comprising a stem-loop at its 3′ end and at least one C16 hydrocarbon chain conjugated to the 5′ terminal nucleotide of the sense strand. In some embodiments, a lipid-conjugated RNAi oligonucleotide comprises a sense strand comprising a tetraloop and at least one C16 hydrocarbon chain conjugated to a nucleotide of the tetraloop.

In some embodiments, a lipid-conjugated RNAi oligonucleotide comprises an antisense strand of 22-24 nucleotides and a sense strand of 20-36 nucleotides described herein, wherein the antisense and sense strands form a duplex region of 20-22 base pairs, wherein the sense strand comprises at least one C16 hydrocarbon chain conjugated to the first nucleotide (Position 1 from 5′>3′) of the sense strand. In some embodiments, a lipid-conjugated RNAi oligonucleotide comprises an antisense strand of 22-24 nucleotides and a sense strand of 20-36 nucleotides described herein, wherein the antisense and sense strands form a duplex region of 20-22 base pairs, wherein the sense strand comprises at least one C16 hydrocarbon chain conjugated to the seventh nucleotide (Position 7 from 5′>3′) of the sense strand. In some embodiments, a lipid-conjugated RNAi oligonucleotide comprises an antisense strand of 22-24 nucleotides and a sense strand of 20-36 nucleotides described herein, wherein the antisense and sense strands form a duplex region of 20-22 base pairs, wherein the sense strand comprises at least one C16 hydrocarbon chain conjugated to the sixteenth nucleotide (Position 16 from 5′ >3′) of the sense strand. In some embodiments, a lipid-conjugated RNAi oligonucleotide comprises an antisense strand of 22-24 nucleotides and a sense strand of 20-36 nucleotides described herein, wherein the antisense and sense strands form a duplex region of 20-22 base pairs, wherein the sense strand comprises at least one C16 hydrocarbon chain conjugated to the twentieth nucleotide (Position 20 from 5′>3′) of the sense strand. In some embodiments, a lipid-conjugated RNAi oligonucleotide comprises an antisense strand of 22-24 nucleotides and a sense strand of 20-36 nucleotides described herein, wherein the antisense and sense strands form a duplex region of 20-22 base pairs, wherein the sense strand comprises at least one C16 hydrocarbon chain conjugated to the twenty-third nucleotide (Position 23 from 5′>3′) of the sense strand. In some embodiments, a lipid-conjugated RNAi oligonucleotide comprises an antisense strand of 22-24 nucleotides and a sense strand of 20-36 nucleotides described herein, wherein the antisense and sense strands form a duplex region of 20-22 base pairs, wherein the sense strand comprises at least one C16 hydrocarbon chain conjugated to the twenty-eighth nucleotide (Position 28 from 5′>3′) of the sense strand. In some embodiments, a lipid-conjugated RNAi oligonucleotide comprises an antisense strand of 22-24 nucleotides and a sense strand of 20-36 nucleotides described herein, wherein the antisense and sense strands form a duplex region of 20-22 base pairs, wherein the sense strand comprises at least one C16 hydrocarbon chain conjugated to the twenty-ninth nucleotide (Position 29 from 5′>3′) of the sense strand. In some embodiments, a lipid-conjugated RNAi oligonucleotide comprises an antisense strand of 22-24 nucleotides and a sense strand of 20-36 nucleotides described herein, wherein the antisense and sense strands form a duplex region of 20-22 base pairs, wherein the sense strand comprises at least one C16 hydrocarbon chain conjugated to the thirtieth nucleotide (Position 30 from 5′>3′) of the sense strand.

In some embodiments, a lipid-conjugated RNAi oligonucleotide for reducing expression of a neuronal target gene comprises the modification pattern of

Sense Strand:
5′-[ademX-Ls][mX][mX][mX][mX][mX][mX][fX][fX][fX]
[fX][mX][mX][mX][mX][mX][mX][mX][mX][mX][mX][mX]
[mX][mX][mX][mX]
[mX][mX][mX][mX][mX][mX][mX][mX][mX][mX]-3′
Hybridized to:
Antisense Strand:
5′-[MePhosphonate-4O-mXs][fXs][fXs][fX][fX]
[mX][fX][mX][mX][fX][mX]
[mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-Ls]=Lipid attached to a nucleotide with a phosphorothioate linkage to the neighboring nucleotide, and [ademX-L]=Lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][ademX-L][fX][fX]
[fX][fX][mX][mX][mX][mX][mX][mX][mX][mX]
[mX][mX][mX][mX][mX][mX][mX][mX][mX]
[mX][mX][mX][mX][mX][mX][mX][mX]-3′
Hybridized to:
Antisense Strand:
5′-[MePhosphonate-4O-mXs][fXs][fXs][fX][fX]
[mX][fX][mX][mX][fX][mX]
[mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-Ls]=Lipid attached to a nucleotide with a phosphorothioate linkage to the neighboring nucleotide, and [ademX-L]=Lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][mX][fX][fX][fX][fX]
[mX][mX][mX][mX][ademX-L]
[mX][mX][mX][mX][mX][mX][mX][mX][mX][mX][mX][mX]
[mX][mX][mX][mX][mX][mX][mX][mX]-3′
Hybridized to:
Antisense Strand:
5′-[MePhosphonate-4O-mXs][fXs][fXs][fX][fX]
[mX][fX][mX][mX][fX][mX]
[mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-Ls]=Lipid attached to a nucleotide with a phosphorothioate linkage to the neighboring nucleotide, and [ademX-L]=Lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][mX][fX][fX][fX]
[fX][mX][mX][mX][mX][mX]
[mX][mX][mX][ademX-L][mX][mX][mX][mX]
[mX][mX][mX][mX]
[mX][mX][mX][mX][mX][mX][mX][mX]-3′
Hybridized to:
Antisense Strand:
5′-[MePhosphonate-4O-mXs][fXs][fXs][fX]
[fX][mX][fX][mX][mX][fX][mX][mX]
[mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-Ls]=Lipid attached to a nucleotide with a phosphorothioate linkage to the neighboring nucleotide, and [ademX-L]=Lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][mX][fX][fX]
[fX][fX][mX][mX][mX][mX][mX][mX]
[mX][mX][mX][mX][mX][ademX-L][mX][mX][mX]
[mX][mX][mX][mX][mX][mX][mX][mX][mX][mX]-3′
Hybridized to:
Antisense Strand:
5′-[MePhosphonate-4O-mXs][fXs][fXs][fX]
[fX][mX][fX][mX][mX][fX][mX][mX]
[mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-Ls]=Lipid attached to a nucleotide with a phosphorothioate linkage to the neighboring nucleotide, and [ademX-L]=Lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][mX][fX][fX][fX][fX][mX][mX]
[mX][mX][mX][mX][mX][mX][mX][mX][mX][mX][mX][mX][mX][mX][ademX-L][mX]
[mX][mX][mX][mX][mX][mX][mX]-3′
Hybridized to:
Antisense Strand:
5′-[MePhosphonate-4O-mXs][fXs][fXs][fX][fX][mX][fX][mX]
[mX][fX][mX][mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-Ls]=Lipid attached to a nucleotide with a phosphorothioate linkage to the neighboring nucleotide, and [ademX-L]=Lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][X][mX][fX][fX][fX][fX][mX][mX]
[mX][mX][mX][mX][mX][mX][mX][mX][mX][mX][mX][mX][mX][mX][mX][ademX-L]
[mX][mX][mX][mX][mX][mX][mX]-3′
Hybridized to:
Antisense Strand:
5′-[MePhosphonate-4O-mXs][fXs][fXs][fX][fX][mX][fX][mX]
[mX][fX][mX][mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-Ls]=Lipid attached to a nucleotide with a phosphorothioate linkage to the neighboring nucleotide, and [ademX-L]=Lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][mX][fX][fX][fX][fX][mX][mX]
[mX][mX][mX][mX][mX][mX][mX][mX][mX][mX][mX][mX][mX][mX][mX][mX]
[ademX-L][mX][mX][mX][mX][mX][mX]-3′
Hybridized to:
Antisense Strand:
5′-[MePhosphonate-4O-mXs][fXs][fXs][fX][fX][mX][fX][mX]
[mX][fX][mX][mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-Ls]=Lipid attached to a nucleotide with a phosphorothioate linkage to the neighboring nucleotide, and [ademX-L]=Lipid attached to a nucleotide.

In some embodiments, a lipid-conjugated RNAi oligonucleotide for reducing expression of a neuronal target gene comprises the modification pattern of

Sense Strand:
5′-[ademX-C16s][mX][mX][mX][mX][mX][mX][fX][fX][fX]
[fX][mX][mX][mX][mX][mX][mX][mX][mX][mX][mX][mX][mX][mX][mX][mX]
[mX][mX][mX][mX][mX][mX][mX][mX][mX][mX]-3′
Hybridized to:
Antisense Strand:
5′-[MePhosphonate-4O-mXs][fXs][fXs][fX][fX][mX][fX][mX]
[mX][fX][mX][mX][nX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-C16s]=C16 lipid attached to a nucleotide with a phosphorothioate linkage to the neighboring nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][ademX-C16][fX][fX][fX][fX]
[mX][mX][mX][mX][mX][mX][mX][mX][mX][mX][mX][mX][mX][mX][mX][mX][mX]
[mX][mX][mX][mX][mX][mX][mX][X]-3′
Hybridized to:
Antisense Strand:
5′-[MePhosphonate-4O-mXs][fXs][fXs][fX][fX][mX][fX][mX]
[mX][fX][mX][mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-C16]=C16 lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][mX][fX][fX][fX][fX][mX][mX]
[mX][mX][ademX-C16][mX][mX][mX][mX][mX][mX][mX][mX][mX][mX][mX][mX]
[mX][mX][mX][mX][mX][mX][mX][mX]-3′
Hybridized to:
Antisense Strand:
5′-[MePhosphonate-4O-mXs][fXs][fXs][fX][fX][mX][fX][mX]
[mX][fX][mX][mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-C16]=C16 lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][mX][fX][fX][fX][fX][mX][mX][mX][mX][mX]
[mX][mX][mX][ademX-C16][mX][mX][mX][mX][mX][mX][mX][mX][mX][mX]
[mX][mX][mX][mX][mX][mX]-3′
Hybridized to:
Antisense Strand:
5′-[MePhosphonate-4O-mXs][fXs][fXs][fX][fX][mX][fX][mX]
[mX][fX][mX][mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-C16]=C16 lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][mX][fX][fX][fX][fX][mX]
[mX][mX][mX][mX][mX][mX][mX][mX][mX][mX][ademX-C16][mX][mX][mX]
[mX][mX][mX][mX][mX][mX][mX][mX][mX][mX]-3′
Hybridized to:
Antisense Strand:
5′-[MePhosphonate-4O-mXs][fXs][fXs][fX][fX][mX][fX][mX]
[mX][fX][mX][mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-C16]=C16 lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][mX][fX][fX][fX][fX][mX][mX]
[mX][mX][mX][mX][mX][mX][mX][mX][mX][mX][mX][mX][mX][mX][ademX-
C16][mX][mX][mX][mX][mX][mX][mX][X]-3′
Hybridized to:
Antisense Strand:
5′-[MePhosphonate-4O-mXs][fXs][fXs][fX][fX][mX][fX][mX]
[mX][fX][mX][mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-C16]=C16 lipid attached to a nucleotide.

In some embodiments, a lipid-conjugated RNAi oligonucleotide for reducing expression of a neuronal target gene comprises the modification pattern of

Sense Strand:
5′-[mXs][mX][mX][mX][mX][mX][mX][fX][fX][fX][fX][mX][mX]
[mX][mX][mX][mX][mX][mX][mX][mX][mX][mX][mX][mX][mX][mX][mX][ademX-
C16][mX][mX][mX][mX][mX][mX][mX]-3′
Hybridized to:
Antisense Strand:
5′-[MePhosphonate-4O-mXs][fXs][fXs][fX][fX][mX][fX][mX]
[mX][fX][mX][mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-C16]=C16 lipid attached to a nucleotide.

### General Methods of Providing the Nucleic Acids and Analogues Thereof

The nucleic acids and analogues thereof comprising lipid conjugate described herein can be made using a variety of synthetic methods known in the art, including standard phosphoramidite methods. Any phosphoramidite synthesis method can be used to synthesize the provided nucleic acids of this disclosure. In certain embodiments, phosphoramidites are used in a solid phase synthesis method to yield reactive intermediate phosphite compounds, which are subsequently oxidized using known methods to produce phosphonate-modified oligonucleotides, typically with a phosphodiester or phosphorothioate internucleotide linkages. The oligonucleotide synthesis of the present disclosure can be performed in either direction: from 5′ to 3′ or from 3′ to 5′ using art known methods.

In certain embodiments, the method for synthesizing a provided nucleic acid comprises (a) attaching a nucleoside or analogue thereof to a solid support via a covalent linkage; (b) coupling a nucleoside phosphoramidite or analogue thereof to a reactive hydroxyl group on the nucleoside or analogue thereof of step (a) to form an internucleotide bond there between, wherein any uncoupled nucleoside or analogue thereof on the solid support is capped with a capping reagent; (c) oxidizing said internucleotide bond with an oxidizing agent; and (d) repeating steps (b) to (c) iteratively with subsequent nucleoside phosphoramidites or analogue thereof to form a nucleic acid or analogue thereof, wherein at least the nucleoside or analogue thereof of step (a), the nucleoside phosphoramidite or analogue thereof of step (b) or at least one of the subsequent nucleoside phosphoramidites or analogues thereof of step (d) comprises a lipid conjugate moiety as described herein. Typically, the coupling, capping/oxidizing steps and optionally, the deprotecting steps, are repeated until the oligonucleotide reaches the desired length and/or sequence, after which it is cleaved from the solid support. In certain embodiments, an oligonucleotide is prepared comprising 1-3 nucleic acid or analogues thereof comprising lipid conjugates units on a tetraloop.

In Scheme A below, where a particular protecting group, leaving group, or transformation condition is depicted, one of ordinary skill in the art will appreciate that other protecting groups, leaving groups, and transformation conditions are also suitable and are contemplated. Certain reactive functional groups (e.g., —N(H)—, —OH, etc.) envisioned in the genera in Scheme A requiring additional protection group strategies are also contemplated and is appreciated by those having ordinary skill in the art. Such groups and transformations are described in detail in March's Advanced Organic Chemistry: Reactions, Mechanisms, and Structure, M. B. Smith and J. March, 5^{th} Edition, John Wiley & Sons, 2001, COMPREHENSIVE ORGANIC TRANSFORMATIONS, (R. C. Larock, 2^{nd} Edition, John Wiley & Sons, 1999), and PROTECTING GROUPS IN ORGANIC SYNTHESIS, (T. W. Greene and P. G. M. Wuts, 3^{rd} edition, John Wiley & Sons, 1999), the entirety of each of which is hereby incorporated herein by reference.

In certain embodiments, nucleic acids, and analogues thereof of the present disclosure are generally prepared according to Scheme A, Scheme A1 and Scheme B set forth below:

### Scheme A: Synthesis of Ligand Conjugated Oligonucleotides of the Disclosure

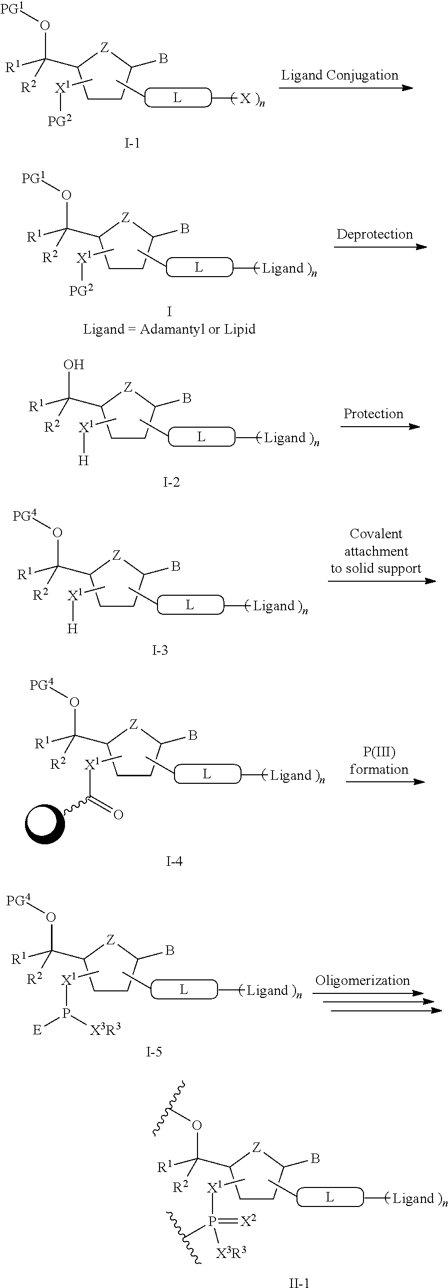

### Scheme A1: Synthesis of Lipid Conjugated Oligonucleotides of the Disclosure

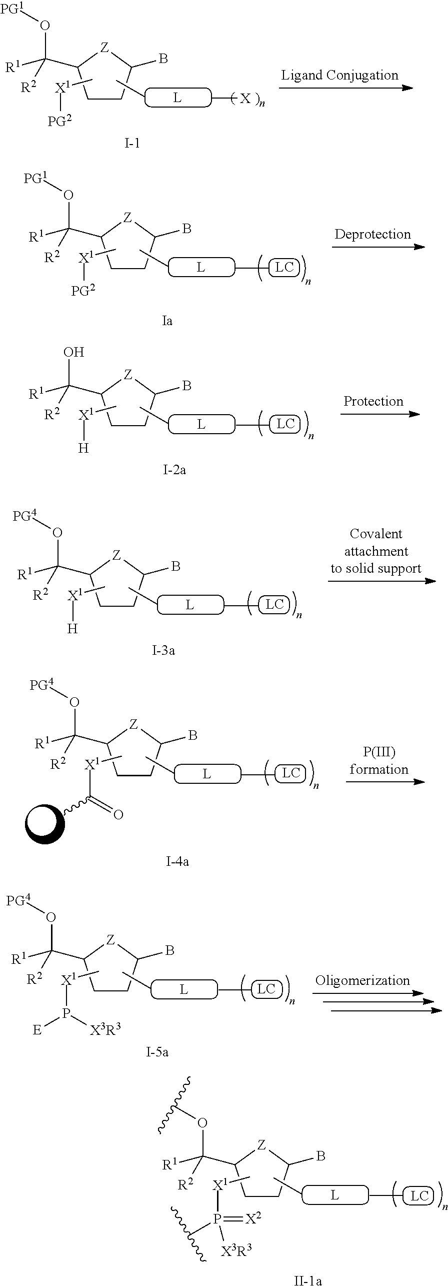

As depicted in Scheme A and Scheme A1 above, a nucleic acid or analogue thereof of formula I-1 is conjugated with one or more ligand/lipophilic compound to form a compound of formula I or Ia comprising one more ligand/lipid conjugates. Typically, conjugation is performed through an esterification or amidation reaction between a nucleic acid or analogue thereof of formula I-1 or I-1a and one or more adamantyl and/or lipophilic compound (e.g., fatty acid) in series or in parallel by known techniques in the art. Nucleic acid or analogue thereof of formula I or Ia can then be deprotected to form a compound of formula I-2 or I-2a and protected with a suitable hydroxyl protecting group (e.g., DMTr) to form a compound of formula I-3 or I-3a. In one aspect, nucleic acid-ligand conjugates of formula I-3 or I-3a can be covalently attached to a solid support (e.g., through a succinic acid linking group) to form a solid support nucleic acid-ligand conjugate or analogue thereof of formula I-4 or I-4a comprising one or more adamantyl and/or lipid conjugate. In another aspect, a nucleic acid-ligand conjugates of formula I-3 or I-3a can react with a P(III) forming reagent (e.g., 2-cyanoethyl N,N-di-isopropylchlorophosphoramidite) to form a nucleic acid or analogue thereof of formula I-5 or I-5a comprising a P(III) group. A nucleic acid-ligand conjugate or analogue thereof of formula I-5 or I-5a can then be subjected to oligomerization forming conditions preformed using known and commonly applied processes to prepare oligonucleotides in the art. For example, the compound of formula I-5 or I-5a is coupled to a solid supported nucleic acid-ligand conjugate or analogue thereof bearing a 5′-hydroxyl group. Further steps can comprise one or more deprotections, couplings, phosphite oxidation, and/or cleavage from the solid support to provide an oligonucleotide of various nucleotide lengths, including one or more lipid conjugate nucleotide units represented by a compound of formula II-1 or II-Ia. Each of B, E, L, ligand, LC, n, PG¹, PG², PG⁴, R¹, R², R³, X, X¹, X², X³, and Z is as defined above and described herein.

### Scheme B: Post-Synthetic Lipid Conjugation of Oligonucleotides of the Disclosure

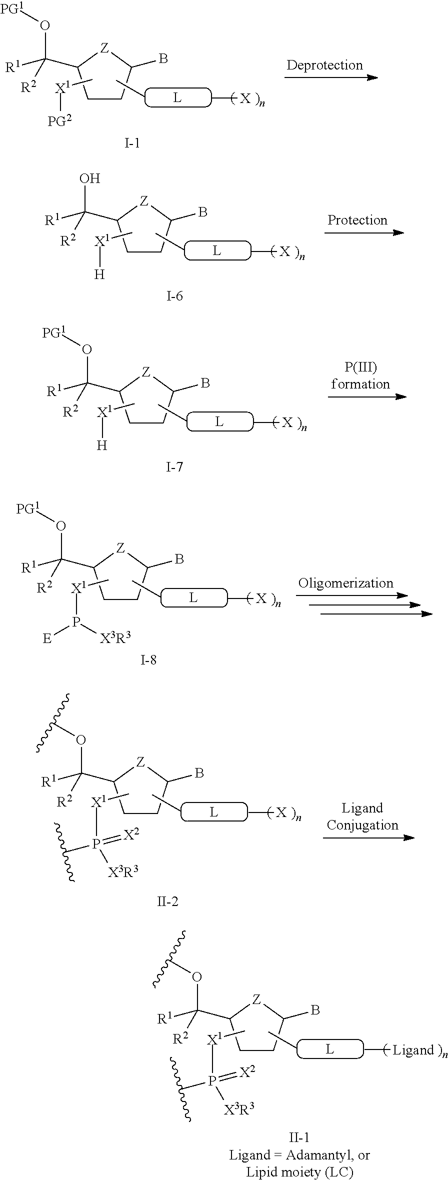

As depicted in Scheme B above, a nucleic acid or analogue thereof of formula I-1 can be deprotected to form a compound of formula I-6, protected with a suitable hydroxyl protecting group (e.g., DMTr) to form a compound of formula I-7, and reacted with a P(III) forming reagent (e.g., 2-cyanoethyl N,N-di-isopropylchlorophosphoramidite) to form a nucleic acid or analogue thereof of formula I-8 comprising a P(III) group. Next, a nucleic acid or analogue thereof of formula I-8 is subjected to oligomerization forming conditions preformed using known and commonly applied processes to prepare oligonucleotides in the art. For example, the compound of formula I-8 is coupled to a solid supported nucleic acid or analogue thereof bearing a 5′-hydroxyl group. Further steps can comprise one or more deprotections, couplings, phosphite oxidation, and/or cleavage from the solid support to provide an oligonucleotide of various nucleotide lengths represented by a compound of formula II-2. An oligonucleotide of formula II-2 can then be conjugated with one or more ligands e.g., adamantyl, or lipophilic compound (e.g., fatty acid) to form a compound of formula II-1 comprising one or more ligand conjugates. Typically, conjugation is performed through an esterification or amidation reaction between a nucleic acid or analogue thereof of formula II-2 and one or more adamantyl or fatty acid in series or in parallel by known techniques in the art. Each of B, E, L, ligand, LC, n, PG¹, PG², PG⁴, R¹, R², R³, X, X¹, X², X³, and Z is as defined above and described herein.

In certain embodiments, nucleic acids, and analogues thereof of the present disclosure are prepared according to Scheme C and Scheme D set forth below:

### Scheme C: Synthesis of Lipid Conjugated Oligonucleotides of the Disclosure

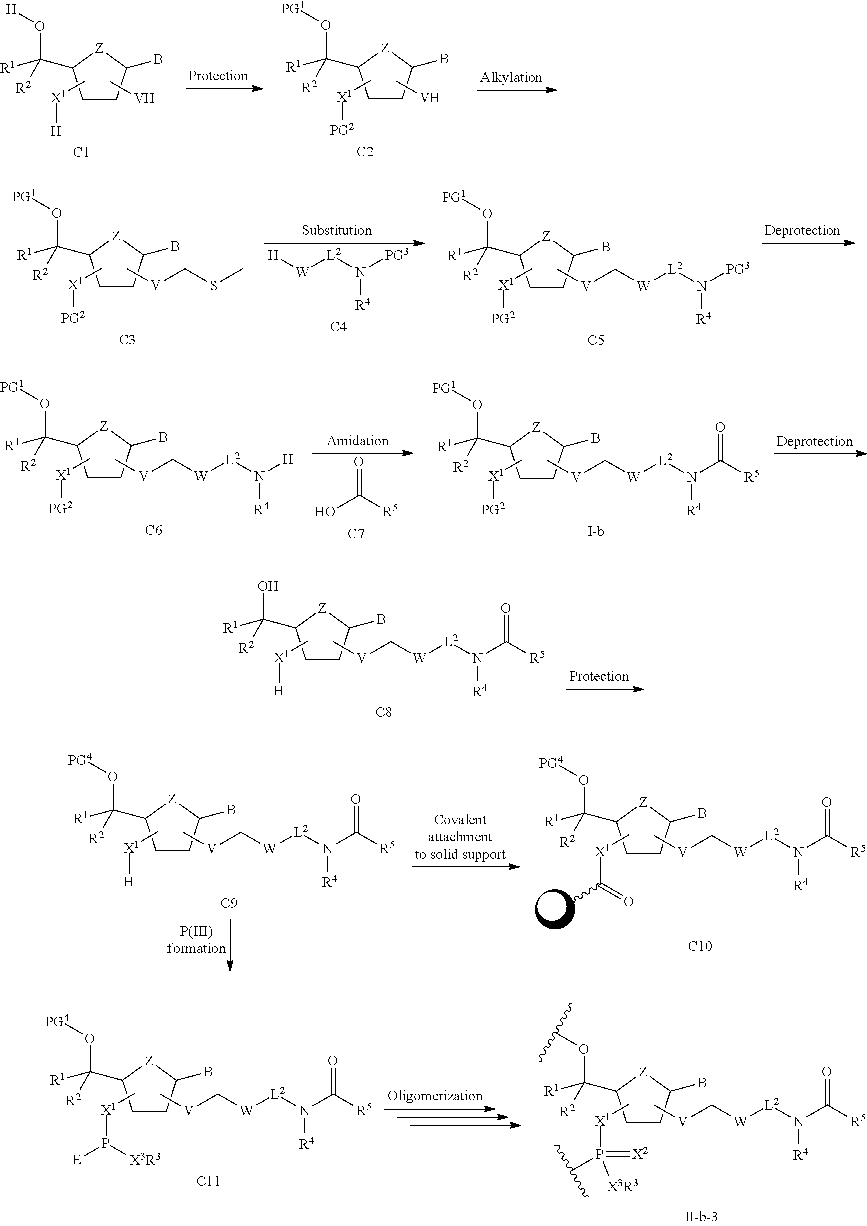

As depicted in Scheme C above, a nucleic acid or analogue thereof of formula C1 is protected to form a compound of formula C2. Nucleic acid or analogue thereof of formula C2 is then alkylated (e.g., using DMSO and acetic acid via the Pummerer rearrangement) to form a monothioacetal compound of formula C3. Next, nucleic acid or analogue thereof of formula C3 is coupled with C4 under appropriate conditions (e.g., mild oxidizing conditions) to form a nucleic acid or analogue thereof of formula C5. Nucleic acid or analogue thereof of formula C5 can then be deprotected to form a compound of formula C6 and coupled with a ligand (adamantyl or lipophilic compound (e.g., a fatty acid)) of formula C7 under appropriate amide forming conditions (e.g., HATU, DIPEA), to form a nucleic acid-ligand conjugate or analogue thereof of formula I-b comprising a lipid conjugate of the disclosure. Nucleic acid-ligand conjugate or analogue thereof of formula I-b can then be deprotected to form a compound of formula C8 and protected with a suitable hydroxyl protecting group (e.g., DMTr) to form a compound of formula C9. In one aspect, nucleic acid, or analogue thereof of formula C9 can be covalently attached to a solid support (e.g., through a succinic acid linking group) to form a solid support nucleic acid-ligand conjugate or analogue thereof of formula C10 comprising a ligand conjugate (adamantyl or lipid moiety) of the disclosure. In another aspect, a nucleic acid-ligand conjugate or analogue thereof of formula C9 can reacted with a P(III) forming reagent (e.g., 2-cyanoethyl N,N-di-isopropylchlorophosphoramidite) to form a nucleic acid-ligand conjugate or analogue thereof of formula C11 comprising a P(III) group. A nucleic acid-ligand conjugate or analogue thereof of formula C11 can then be subjected to oligomerization forming conditions preformed using known and commonly applied processes to prepare oligonucleotides in the art. For example, the compound of formula C11 is coupled to a solid supported nucleic acid-ligand conjugate or analogue thereof bearing a 5′-hydroxyl group. Further steps can comprise one or more deprotections, couplings, phosphite oxidation, and/or cleavage from the solid support to provide an oligonucleotide of various nucleotide lengths, including one or more adamantyl and/or lipid conjugate nucleotide units represented by a compound of formula II-b-3. Each of B, E, L², PG¹, PG², PG³, PG⁴, R¹, R², R³, R⁴, R⁵, X¹, X², X³, V, W, and Z is as defined above and described herein.

### Scheme D: Post-Synthetic Lipid Conjugation of Oligonucleotides of the Disclosure

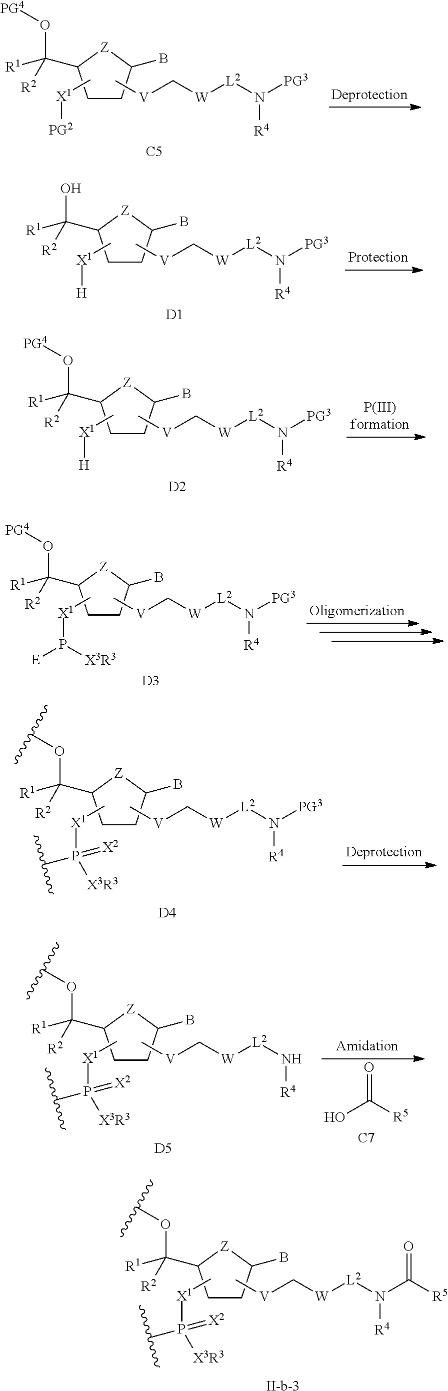

Each of B, E, L², PG¹, PG², PG³, PG⁴, R¹, R², R³, R⁴, R⁵, X¹, X², X³, V, W, and Z is as defined above and described herein. As depicted in Scheme D above, a nucleic acid or analogue thereof of formula C5 can be selectively deprotected to form a compound of formula D1, protected with a suitable hydroxyl protecting group (e.g., DMTr) to form a compound of formula D2, and reacted with a P(III) forming reagent (e.g., 2-cyanoethyl N,N-di-isopropylchlorophosphoramidite) to form a nucleic acid or analogue thereof of formula D3. Next, a nucleic acid or analogue thereof of formula D3 is subjected to oligomerization forming conditions preformed using known and commonly applied processes to prepare oligonucleotides in the art. For example, the compound of formula D3 is coupled to a solid supported nucleic acid or analogue thereof bearing a 5′-hydroxyl group. Further steps can comprise one or more deprotections, couplings, phosphite oxidation, and/or cleavage from the solid support to provide an oligonucleotide of various nucleotide lengths, represented by a compound of formula D4. An oligonucleotide of formula D4 can then be deprotected to form a compound of formula D5 and coupled with a hydrophobic ligand (e.g., adamantyl or a lipophilic moiety) to form a compound of formula C7 (e.g., adamantyl or a fatty acid) under appropriate amide forming conditions (e.g., HATU, DIPEA), to form an oligonucleotide of formula II-b-3 comprising a ligand (e.g., adamantyl or a fatty acid) conjugate of the disclosure.

One of skill in the art will appreciate that various functional groups present in the nucleic acid or analogues thereof of the disclosure such as aliphatic groups, alcohols, carboxylic acids, esters, amides, aldehydes, halogens, and nitriles can be interconverted by techniques well known in the art including, but not limited to reduction, oxidation, esterification, hydrolysis, partial oxidation, partial reduction, halogenation, dehydration, partial hydration, and hydration. See for example, “MARCH'S ADVANCED ORGANIC CHEMISTRY”, (5^{th} Ed., Ed.: Smith, M. B. and March, J., John Wiley & Sons, New York: 2001), the entirety of each of which is herein incorporated by reference. Such interconversions may require one or more of the aforementioned techniques, and certain methods for synthesizing the provided nucleic acids of the disclosure are described below in the Exemplification.

In some embodiments, the present disclosure provides a method for preparing an oligonucleotide comprising one or more lipid conjugate, said lipid conjugate unit represent by formula II-a-1:

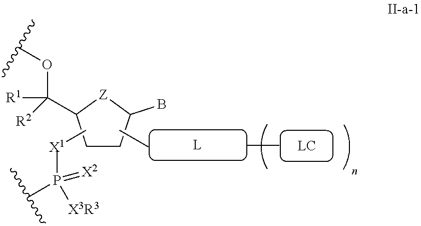

or a pharmaceutically acceptable salt thereof, comprising the steps of:
(a) providing a nucleic acid or analogue thereof of formula I-5a:

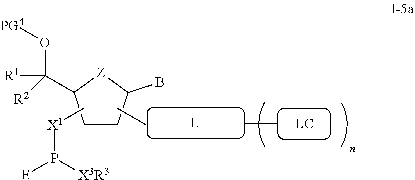

or salt thereof, and
(b) oligomerizing said compound of formula I-5a to form a compound of formula II-1a, wherein each of B, E, L, LC, n, PG⁴, R¹, R², R³, X, X¹, X², X³, E, and Z is as defined above and described herein.

In step (b) above, oligomerizing refers to preforming oligomerization forming conditions using known and commonly applied processes to prepare oligonucleotides in the art. For example, the compound of formula I-5a is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula II-1a comprising a lipid conjugate of the disclosure.

In some embodiments, the present disclosure provides a method for preparing an oligonucleotide comprising one or more lipid conjugate, further comprising preparing a nucleic acid or analogue thereof of formula I-5a:

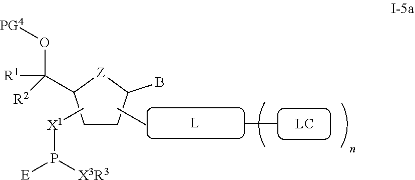

or a salt thereof, comprising the steps of:
(a) providing a nucleic acid or analogue thereof of formula Ia:

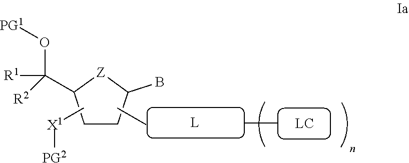

or salt thereof,
(b) deprotecting said nucleic acid or analogue thereof of formula Ia to form a compound of formula I-2a:

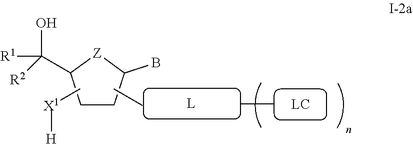

or salt thereof,
(c) protecting said nucleic acid or analogue thereof of formula I-2 to form a compound of formula I-3a:

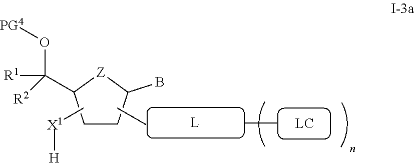

or salt thereof, and
(d) treating said nucleic acid or analogue thereof of formula I-3a with a P(III) forming reagent to form a nucleic acid or analogue thereof of formula I-5a, wherein each of B, E, L, LC, n, PG⁴, R¹, R², R³, X, X¹, X², X³, E, and Z is as defined above and described herein.

In step (b) above, PG¹ and PG² of a compound of formula Ia comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula I-2a is protected with a suitable hydroxyl protecting group. In certain embodiments, the protecting group PG⁴ used for protection of the 5′-hydroxyl group of a compound of formula I-2a includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula I-3a is treated with a P(III) forming reagent to afford a compound of formula I-5a. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula I-3a is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide comprising one or more lipid conjugates, further comprising preparing a nucleic acid-lipid conjugate or analogue thereof of formula Ia:

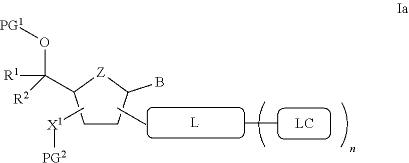

or a salt thereof, comprising the steps of:
(a) providing a nucleic acid or analogue thereof of formula I-1:

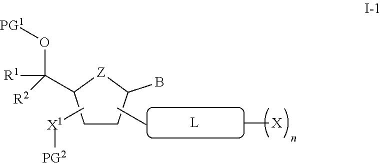

or salt thereof, and,
(b) conjugating one or more lipophilic compounds to a nucleic acid or analogue thereof of formula I-1 to form a nucleic acid or analogue thereof of formula Ia comprising one or more lipid conjugates, wherein: each of B, E, L, LC, n, PG¹, PG², R1, R², X, X¹, and Z is as defined above and described herein.

In step (b) above, a nucleic acid or analogue thereof of formula I-1a is conjugated with one or more lipophilic compounds to form a compound of formula Ia comprising one more lipid conjugates of the disclosure. Typically, conjugation is performed through an esterification or amidation reaction between a nucleic acid or analogue thereof of formula I-1a and one or more fatty acids in series or in parallel by known techniques in the art. In certain embodiments, conjugation is performed under suitable amide forming conditions to afford a compound of formula I comprising one more lipid conjugates. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. Alternatively, conjugation of a lipophilic compound can be accomplished by any one of the cross-coupling technologies described in Table A herein.

In some embodiments, the present disclosure provides a method for preparing an oligonucleotide comprising one or more lipid conjugate, said lipid conjugate unit represent by formula II-1:

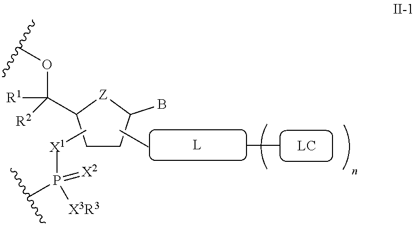

or a pharmaceutically acceptable salt thereof, comprising the steps of:
(a) providing an oligonucleotide of formula II-2:

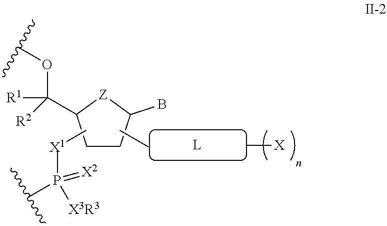

or salt thereof, and,
(b) conjugating one or more lipophilic compounds to an oligonucleotide of formula II-2 to form an oligonucleotide of formula II-1 comprising one or more lipid conjugates. In step (b) above, an oligonucleotide of formula II-2 is conjugated with one or more lipophilic compounds to form an oligonucleotide of formula II-1 comprising one more lipid conjugates of the disclosure. Typically, conjugation is performed through an esterification or amidation reaction between an oligonucleotide of formula II-2 and one or more fatty acids in series or in parallel by known techniques in the art. In certain embodiments, conjugation is performed under suitable amide forming conditions to afford an oligonucleotide of formula II-1 comprising one more lipid conjugates. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. Alternatively, conjugation of a lipophilic compound can be accomplished by any one of the cross-coupling technologies described in Table A herein.

In some embodiments, the present disclosure provides a method for preparing an oligonucleotide comprising a unit represent by formula II-2:

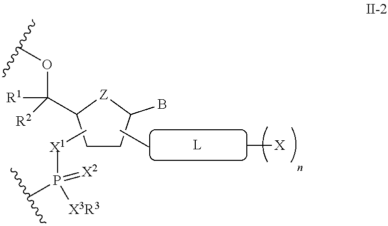

or a pharmaceutically acceptable salt thereof, comprising the steps of:
(a) providing a nucleic acid or analogue thereof of formula I-8:

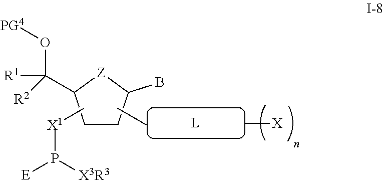

or salt thereof, and
(b) oligomerizing said compound of formula I-8 to form a compound of formula II-2.

In step (b) above, oligomerizing refers to preforming oligomerization forming conditions using known and commonly applied processes to prepare oligonucleotides in the art. For example, the compound of formula I-8 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula II-2.

In some embodiments, the present disclosure provides a method for preparing a nucleic acid or analogue thereof comprising one or more lipid conjugate, further comprising preparing a nucleic acid or analogue thereof of formula I-8:

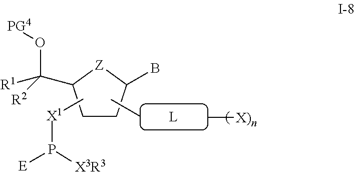

or a salt thereof, comprising the steps of:
(a) providing a nucleic acid or analogue thereof of formula I-1:

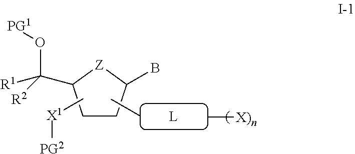

or salt thereof,
(b) deprotecting said nucleic acid or analogue thereof of formula I-1 to form a compound of formula I-6:

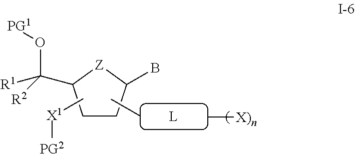

or salt thereof,
(c) protecting said nucleic acid or analogue thereof of formula I-6 to form a compound of formula I-7:

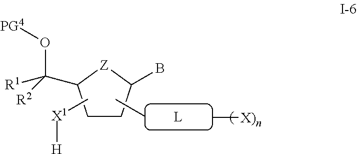

or salt thereof, and
(d) treating said nucleic acid or analogue thereof of formula I-7 with a P(III) forming reagent to form a nucleic acid or analogue thereof of formula I-8, In step (b) above, PG¹ and PG² of a compound of formula I-1 comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula I-6 is protected with a suitable hydroxyl protecting group. In certain embodiments, the protecting group PG⁴ used for protection of the 5′-hydroxyl group of a compound of formula I-6 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula I-7 is treated with a P(III) forming reagent to afford a compound of formula I-8. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula I-7 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide-ligand conjugate comprising one or more adamantyl and/or lipid moieties, said conjugate unit represented by formula II-b-3:

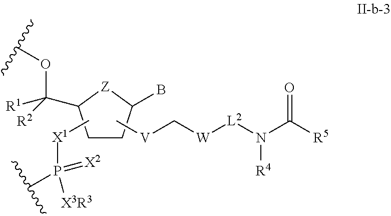

or a pharmaceutically acceptable salt thereof, comprising the steps of:
(a) providing a nucleic acid-ligand conjugate or analogue thereof of formula C11:

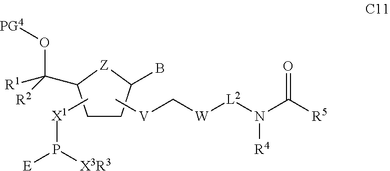

or salt thereof, and
(b) oligomerizing said compound of formula C11 to form a compound of formula II-b-3, In step (b) above, oligomerizing refers to preforming oligomerization forming conditions using known and commonly applied processes to prepare oligonucleotides in the art. For example, the compound of formula C11 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide-ligand conjugate of various nucleotide lengths, with one or more nucleic acid-ligand conjugate units, wherein each unit is represented by a compound of formula II-b-3 comprising an adamantyl or lipid moiety of the disclosure.

In some embodiments, the method for preparing an oligonucleotide of formula II-b-3 comprising one or more lipid conjugate, further comprises preparing a nucleic acid-ligand conjugate or analogue thereof of formula C11:

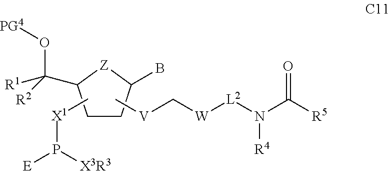

or a salt thereof, comprising the steps of:
(a) providing a nucleic acid-ligand conjugate or analogue thereof of formula I-b:

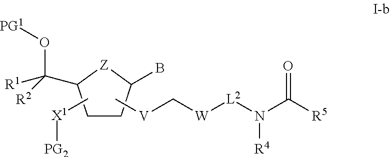

or salt thereof,
(b) deprotecting said nucleic acid-ligand conjugate or analogue thereof of formula I-b to form a compound of formula C8:

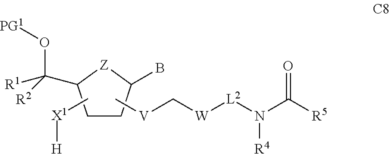

or salt thereof,
(c) protecting said nucleic acid-ligand conjugate or analogue thereof of formula C8 to form a compound of formula C9:

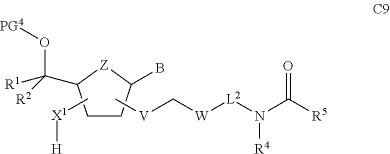

or salt thereof, and
(d) treating said nucleic acid-ligand conjugate or analogue thereof of formula C9 with a P(III) forming reagent to form a nucleic acid or analogue thereof of formula C11. In step (b) above, PG¹ and PG² of a compound of formula I-b comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula C8 is protected with a suitable hydroxyl protecting group. In certain embodiments, the protecting group PG⁴ used for protection of the 5′-hydroxyl group of a compound of formula C8 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula C9 is treated with a P(III) forming reagent to afford a compound of formula C11. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula C9 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide-ligand conjugate of formula II-b-3 comprising one or more nucleic acid-ligand conjugate units each comprising one or more adamantyl or lipid moieties, further comprising preparing a nucleic acid-ligand conjugate or analogue thereof of formula I-b:

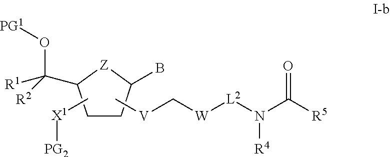

or a salt thereof, comprising the steps of
(a) providing a nucleic acid-ligand conjugate or analogue thereof of formula C6:

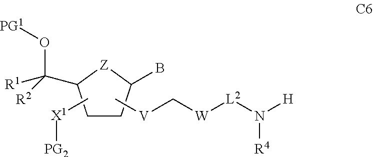

or salt thereof, and,
(b) conjugating a lipophilic compound to a nucleic acid or analogue thereof of formula C6 to form a nucleic acid-ligand conjugate or analogue thereof of formula I-b comprising one or more adamantyl and/or lipid conjugates. In step (b) above, conjugation is performed under suitable amide forming conditions to afford a compound of formula I-b comprising an adamantyl and/or lipid conjugate. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the amide forming conditions comprise HATU and DIPEA or TEA.

In certain embodiments, a nucleic acid-ligand conjugate or analogue thereof of formula C6 is provided in salt form (e.g., a fumarate salt) and is first converted to the free base (e.g., using sodium bicarbonate) before preforming the conjugation step.

In some embodiments, the present disclosure provides a method for preparing an oligonucleotide-ligand conjugate of formula II-b-3 comprising one or more nucleic acid-ligand conjugate units, further comprises preparing a nucleic acid-ligand conjugate or analogue thereof of formula C6:

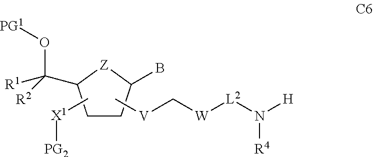

or a salt thereof, comprising the steps of:
(a) providing a nucleic acid or analogue thereof of formula C1:

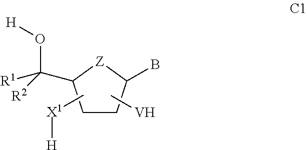

or salt thereof, and,
(b) protecting said nucleic acid or analogue thereof of formula C1 to form a compound of formula C2:

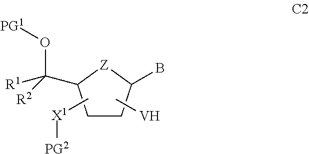

or salt thereof,
(c) alkylating said nucleic acid or analogue thereof of formula C2 to form a compound of formula C3:

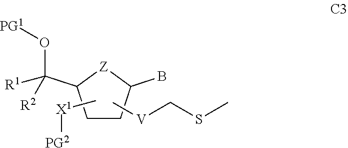

or salt thereof,
(d) substituting said nucleic acid or analogue thereof of formula C3 with a compound of formula C4:

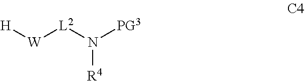

or salt thereof, to form a compound of formula C5:

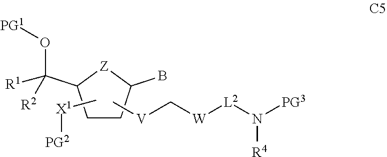

or salt thereof,
(e) deprotecting said nucleic acid or analogue thereof of formula C5 to form a nucleic acid-ligand conjugate or analogue thereof of formula C6. In step (b) above, PG¹ and PG² groups of formula C2 are taken together with their intervening atoms to form a cyclic diol protecting group, such as a cyclic acetal or ketal. Such groups include methylene, ethylidene, benzylidene, isopropylidene, cyclohexylidene, and cyclopentylidene, silylene derivatives such as di-t-butylsilylene and 1,1,3,3-tetraisopropylidisiloxanylidene, a cyclic carbonate, a cyclic boronate, and cyclic monophosphate derivatives based on cyclic adenosine monophosphate (i.e., cAMP). In certain embodiments, the cyclic diol protection group is 1,1,3,3-tetraisopropylidisiloxanylidene prepared from the reaction of a diol of formula C1 and 1,3-dichloro-1,1,3,3-tetraisopropyldisiloxane under basic conditions.

In step (c) above, a nucleic acid or analogue thereof of formula C2 is alkylated with a mixture of DMSO and acetic anhydride under acidic conditions. In certain embodiments, when -V-H is a hydroxyl group, the mixture of DMSO and acetic anhydride in the presence of acetic acid forms (methylthio)methyl acetate in situ via the Pummerer rearrangement which then reacts with the hydroxyl group of the nucleic acid or analogue thereof of formula C2 to provide a monothioacetal functionalized fragment nucleic acid or analogue thereof of formula C3.

In step (d) above, substitution of the thiomethyl group of a nucleic acid or analogue thereof of formula C3 using a nucleic acid or analogue thereof of formula C4 affords a nucleic acid or analogue thereof of formula C4. In certain embodiments, substitution occurs under mild oxidizing and/or acidic conditions. In some embodiments, V is oxygen. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

In step (e) above, removal of PG³ and optionally R⁴ (when R⁴ is a suitable amine protecting group) of a nucleic acid-ligand conjugate or analogue thereof of formula C5 affords a nucleic acid-ligand conjugate or analogue thereof of formula C6 or a salt thereof. In some embodiments, PG³ and/or R⁴ comprise carbamate derivatives that can be removed under acidic or basic conditions. In certain embodiments, the protecting groups (e.g., both PG³ and R⁴ or either of PG³ or R⁴ independently) of a nucleic acid-ligand conjugate or analogue thereof of formula C5 are removed by acid hydrolysis. It will be appreciated that upon acid hydrolysis of the protecting groups of a nucleic acid-ligand conjugate or analogue thereof of formula C5, a salt of formula C6 thereof is formed. For example, when an acid-labile protecting group of a nucleic acid-ligand conjugate or analogue thereof of formula C5 is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a nucleic acid or analogue thereof of formula C6 are contemplated.

In other embodiments, the protecting groups (e.g., both PG³ and R⁴ or either of PG³ or R⁴ independently) of a nucleic acid or analogue thereof of formula C5 are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-diazabicyclo[5.4.0]undec-7-ene (DBU). In certain embodiments, a nucleic acid-ligand conjugate or analogue thereof of formula C5 is deprotected under basic conditions followed by treating with an acid to form a salt of formula C6. In certain embodiments, the acid is fumaric acid the salt of formula C6 is the fumarate.

In some embodiments, the present disclosure provides a method for preparing an oligonucleotide-ligand conjugate comprising one or more nucleic acid-ligand conjugate, said nucleic acid-ligand conjugate unit represented by formula II-b-3:

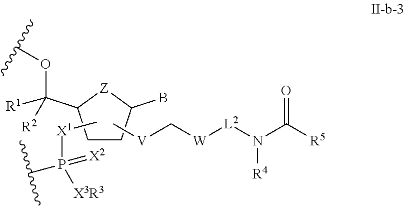

or a pharmaceutically acceptable salt thereof, comprising the steps of:
(a) providing an oligonucleotide of formula D5:

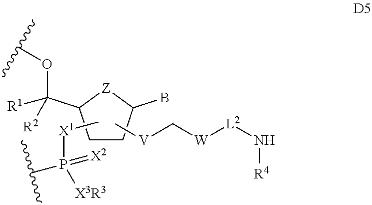

or salt thereof, and,
(b) conjugating one or more adamantyl or lipophilic compounds to an oligonucleotide of formula D5 to form an oligonucleotide-ligand conjugate of formula II-b-3 comprising one or more nucleic acid-ligand conjugate units. In step (b) above, conjugation is performed under suitable amide forming conditions to afford a compound of formula D5 comprising an adamantyl or lipid conjugate. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the amide forming conditions comprise HATU and DIPEA or TEA.

In some embodiments, the present disclosure provides a method for preparing an oligonucleotide-ligand conjugate comprising a unit represent by formula D5:

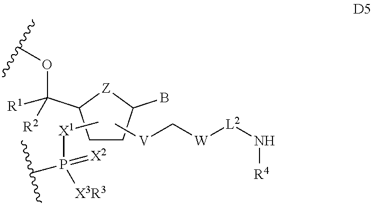

or a salt thereof, comprising the steps of
(a) providing a nucleic acid-ligand conjugate or analogue thereof of formula D4:

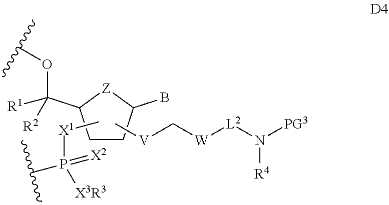

or salt thereof, and
(b) deprotecting said compound of formula D4 to form a compound of formula D5. In step (b) above, removal of PG³ and optionally R⁴ (when R⁴ is a suitable amine protecting group) of an oligonucleotide of formula D4 affords an oligonucleotide-ligand conjugate of formula D5 or a salt thereof. In some embodiments, PG³ and/or R⁴ comprise carbamate derivatives that can be removed under acidic or basic conditions. In certain embodiments, the protecting groups (e.g., both PG³ and R⁴ or either of PG³ or R⁴ independently) of an oligonucleotide-ligand conjugate of formula D4 are removed by acid hydrolysis. It will be appreciated that upon acid hydrolysis of the protecting groups of an oligonucleotide-ligand conjugate of formula D4, a salt of formula D5 thereof is formed. For example, when an acid-labile protecting group of an oligonucleotide of formula D4 is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a nucleic acid-ligand conjugate unit or analogue thereof of formula D5 are contemplated.

In other embodiments, the protecting groups (e.g., both PG³ and R⁴ or either of PG³ or R⁴ independently) of an oligonucleotide-ligand conjugate of formula D4 are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-diazabicyclo[5.4.0]undec-7-ene (DBU).

In some embodiments, the present disclosure provides a method for preparing an oligonucleotide-ligand conjugate comprising one or more nucleic acid-ligand conjugate unit with one or more adamantyl and/or lipid moiety, said conjugate unit represented by formula D4:

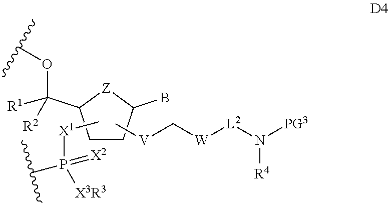

or a pharmaceutically acceptable salt thereof, comprising the steps of:
(a) providing a nucleic acid or analogue thereof of formula D3:

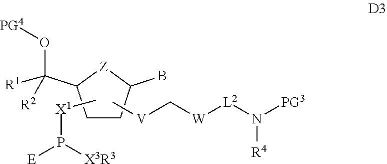

or salt thereof, and
(b) oligomerizing said compound of formula D3 to form a compound of formula D4, In step (b) above, oligomerizing refers to preforming oligomerization forming conditions using known and commonly applied processes to prepare oligonucleotides in the art. For example, the nucleic acid or analogue thereof of formula D3 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula D4 comprising an adamantyl or lipid conjugate of the disclosure.

In some embodiments, the present disclosure provides a method for preparing a nucleic acid or analogue thereof comprising one or more lipid conjugate, further comprising preparing a nucleic acid or analogue thereof of formula D3:

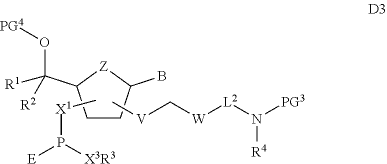

or a salt thereof, comprising the steps of:
(a) providing a nucleic acid or analogue thereof of formula C5:

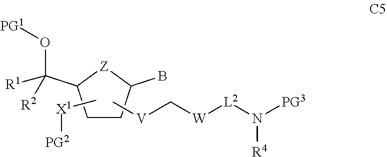

or salt thereof,
(b) deprotecting said nucleic acid or analogue thereof of formula C5 to form a compound of formula D1:

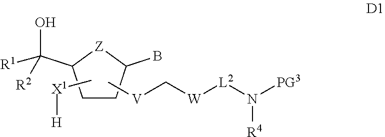

or salt thereof,
(c) protecting said nucleic acid or analogue thereof of formula D1 to form a nucleic acid or analogue thereof of formula D2:

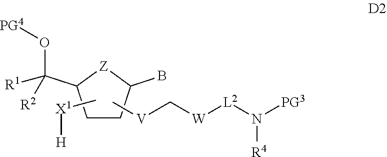

or salt thereof, and
(d) treating said nucleic acid or analogue thereof of formula D2 with a P(III) forming reagent to form a nucleic acid or analogue thereof of formula D3. In step (b) above, PG¹ and PG² of a nucleic acid or analogue thereof of formula C5 comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a nucleic acid or analogue thereof of formula D1 is protected with a suitable hydroxyl protecting group. In certain embodiments, the protecting group PG⁴ used for protection of the 5′-hydroxyl group of a compound of formula D1 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a nucleic acid or analogue thereof of formula D2 is treated with a P(III) forming reagent to afford a compound of formula D3. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula D2 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

### Formulations

Various formulations have been developed to facilitate oligonucleotide use. For example, oligonucleotides (e.g., lipid-conjugated RNAi oligonucleotides) can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., lipid-conjugated RNAi oligonucleotides) reduce the expression of a target mRNA (e.g., a target mRNA expressed in a neuron of the CNS). Such compositions can be suitably formulated such that when administered to a subject, either into the immediate environment of a target cell or systemically, a sufficient portion of the oligonucleotides enter the cell to reduce target gene expression. Any variety of suitable oligonucleotide formulations can be used to deliver oligonucleotides for the reduction of neuronal target gene expression as disclosed herein. In some embodiments, an oligonucleotide is formulated in buffer solutions such as phosphate buffered saline solutions, liposomes, micellar structures, and capsids.

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin). Likewise, the oligonucleotides herein may be provided in the form of their free acids.

In some embodiments, a pharmaceutical composition is formulated to be compatible with its intended route of administration. Examples of routes of administration include parenteral (e.g., intravenous, intramuscular, intraperitoneal, intradermal, subcutaneous, intrathecal), oral (e.g., inhalation), transdermal (e.g., topical), transmucosal and rectal administration. In some embodiments, a pharmaceutical composition is formulated for delivery to the central nervous system (e.g., intrathecal, epidural). In some embodiments, a pharmaceutical composition is formulated for delivery to the eye (e.g., ophthalmic, intraocular, subconjunctival, intravitreal, retrobulbar, intracameral).

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., an lipid-conjugated RNAi oligonucleotide herein) or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### METHODS OF USE

### Reducing Target Gene Expression

In some embodiments, the disclosure provides methods for contacting or delivering to a cell or population of cells an effective amount of any of the lipid-conjugated RNAi oligonucleotides herein to reduce expression of a target gene in a neuron in the CNS. In some embodiments, expression of a neuronal target gene is reduced in a region of the CNS. In some embodiments, regions of the CNS include, but are not limited to, cerebrum, prefrontal cortex, frontal cortex, motor cortex, temporal cortex, parietal cortex, occipital cortex, somatosensory cortex, hippocampus, caudate, striatum, globus pallidus, thalamus, midbrain, tegmentum, substantia nigra, pons, brainstem, cerebellar white matter, cerebellum, dentate nucleus, medulla, cervical spinal cord, thoracic spinal cord, lumbar spinal cord, cervical dorsal root ganglion, thoracic dorsal root ganglion, lumbar dorsal root ganglion, sacral dorsal root ganglion, nodose ganglia, femoral nerve, sciatic nerve, sural nerve, amygdala, hypothalamus, putamen, corpus callosum, and cranial nerve. In some embodiments, the region of the CNS is selected from the lumbar spinal cord, lumbar dorsal root ganglion, medulla, hippocampus, somatosensory cortex, frontal cortex, and a combination thereof. In some embodiments, the region of the CNS is selected from the spinal cord, lumbar spinal cord, lumbar dorsal root ganglion, thoracic spinal cord, cervical spinal cord, medulla, hippocampus, somatosensory cortex, frontal cortex, and a combination thereof. In some embodiments, a lipid-conjugated RNAi oligonucleotide described herein reduces expression of a target gene in a neuron in the spinal cord. In some embodiments, a lipid-conjugated RNAi oligonucleotide described herein reduces expression of a target gene in a neuron in the lumbar spinal cord. In some embodiments, a lipid-conjugated RNAi oligonucleotide described herein reduces expression of a target gene in a neuron in the thoracic spinal cord. In some embodiments, a lipid-conjugated RNAi oligonucleotide described herein reduces expression of a target gene in a neuron of the cervical spinal cord. In some embodiments, a lipid-conjugated RNAi oligonucleotide described herein reduces expression of a target gene in a neuron in lumbar dorsal root ganglion. In some embodiments, a lipid-conjugated RNAi oligonucleotide described herein reduces expression of a target gene in a neuron in the medulla. In some embodiments, a lipid-conjugated RNAi oligonucleotide described herein reduces expression of a target gene in a neuron in the hippocampus. In some embodiments, a lipid-conjugated RNAi oligonucleotide described herein reduces expression of a target gene in a neuron in the somatosensory cortex. In some embodiments, a lipid-conjugated RNAi oligonucleotide described herein reduces expression of a target gene in a neuron in the frontal cortex.

In some embodiments, a lipid-conjugated RNAi oligonucleotide comprising a stem-loop as described herein reduces expression of a target gene in a neuron in the spinal cord. In some embodiments, a lipid-conjugated RNAi oligonucleotide comprising a stem-loop as described herein reduces expression of a target gene in a neuron in the lumbar spinal cord. In some embodiments, a lipid-conjugated RNAi oligonucleotide comprising a stem-loop as described herein reduces expression of a target gene in a neuron in the thoracic spinal cord. In some embodiments, a lipid-conjugated RNAi oligonucleotide comprising a stem-loop as described herein reduces expression of a target gene in a neuron in the cervical spinal cord. In some embodiments, a lipid-conjugated RNAi oligonucleotide comprising a stem-loop as described herein reduces expression of a target gene in a neuron in the lumbar dorsal root ganglion.

In some embodiments, a lipid-conjugated RNAi oligonucleotide comprising a stem-loop as described herein reduces expression of a target gene in a neuron only in the spinal cord. In some embodiments, a lipid-conjugated RNAi oligonucleotide comprising a stem-loop as described herein reduces expression of a target gene in a neuron only in the lumbar spinal cord. In some embodiments, a lipid-conjugated RNAi oligonucleotide comprising a stem-loop as described herein reduces expression of a target gene in a neuron only in the thoracic spinal cord. In some embodiments, a lipid-conjugated RNAi oligonucleotide comprising a stem-loop as described herein reduces expression of a target gene in a neuron only in the cervical spinal cord. In some embodiments, a lipid-conjugated RNAi oligonucleotide comprising a stem-loop as described herein reduces expression of a target gene in a neuron only in the lumbar dorsal root ganglion.

In some embodiments, a lipid-conjugated RNAi oligonucleotide comprising a stem-loop as described herein, reduces expression of a target gene in a neuron at least in the spinal cord. In some embodiments, a lipid-conjugated RNAi oligonucleotide comprising a stem-loop as described herein, reduces expression of a target gene in a neuron at least in the lumbar spinal cord. In some embodiments, a lipid-conjugated RNAi oligonucleotide comprising a stem-loop as described herein, reduces expression of a target gene in a neuron at least in the thoracic spinal cord. In some embodiments, a lipid-conjugated RNAi oligonucleotide comprising a stem-loop as described herein, reduces expression of a target gene in a neuron at least in the cervical spinal cord. In some embodiments, a lipid-conjugated RNAi oligonucleotide comprising a stem-loop as described herein, reduces expression of a target gene in a neuron at least in the lumbar dorsal root ganglion.

In some embodiments, a lipid-conjugated RNAi oligonucleotide comprising a stem-loop as described herein, reduces expression of a target gene in a neuron in the spinal cord relative to other tissues of the CNS (e.g., medulla, hippocampus, and frontal cortex). In some embodiments, a lipid-conjugated RNAi oligonucleotide comprising a stem-loop as described herein, reduces expression of a target gene in a neuron in the lumbar spinal cord relative to other tissues of the CNS (e.g., medulla, hippocampus, and frontal cortex). In some embodiments, a lipid-conjugated RNAi oligonucleotide comprising a stem-loop as described herein, reduces expression of a target gene in a neuron in the thoracic spinal cord relative to other tissues of the CNS (e.g., medulla, hippocampus, and frontal cortex). In some embodiments, a lipid-conjugated RNAi oligonucleotide comprising a stem-loop as described herein, reduces expression of a target gene in a neuron in the cervical spinal cord relative to other tissues of the CNS (e.g., medulla, hippocampus, and frontal cortex). In some embodiments, a lipid-conjugated RNAi oligonucleotide comprising a stem-loop as described herein, reduces expression of a target gene in a neuron in the lumbar dorsal root ganglion relative to other tissues of the CNS (e.g., medulla, hippocampus, and frontal cortex).

In some embodiments, expression of a neuronal target gene in the spinal cord of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in other CNS tissue.

In some embodiments, a lipid-conjugated RNAi oligonucleotide comprising a stem-loop as described herein reduces target gene expression in a neuron of the spinal cord about 1%, about 5%, about 10%, about 15%, about 20%, about 25%, about 30%, about 35%, about 40%, about 45%, about 50% or, about 55%, about 60%, about 70%, about 80%, or about 90% lower relative to reduced gene expression by the lipid-conjugated RNAi oligonucleotide in one or more tissues of the CNS.

In some embodiments, expression of a neuronal target gene in the lumbar spinal cord of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in other CNS tissue.

In some embodiments, a lipid-conjugated RNAi oligonucleotide comprising a stem-loop as described herein reduces target gene expression in a neuron of the lumbar spinal cord about 1%, about 5%, about 10%, about 15%, about 20%, about 25%, about 30%, about 35%, about 40%, about 45%, about 50% or, about 55%, about 60%, about 70%, about 80%, or about 90% lower relative to reduced gene expression by the lipid-conjugated RNAi oligonucleotide in one or more tissues of the CNS.

In some embodiments, expression of a neuronal target gene in the cervical spinal cord of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in other CNS tissue.

In some embodiments, a lipid-conjugated RNAi oligonucleotide comprising a stem-loop as described herein reduces target gene expression in a neuron of the cervical spinal cord about 1%, about 5%, about 10%, about 15%, about 20%, about 25%, about 30%, about 35%, about 40%, about 45%, about 50% or, about 55%, about 60%, about 70%, about 80%, or about 90% lower relative to reduced gene expression by the lipid-conjugated RNAi oligonucleotide in one or more tissues of the CNS.

In some embodiments, expression of a neuronal target gene in the thoracic spinal cord of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in other CNS tissue.

In some embodiments, a lipid-conjugated RNAi oligonucleotide comprising a stem-loop as described herein reduces target gene expression in a neuron of the thoracic spinal cord about 1%, about 5%, about 10%, about 15%, about 20%, about 25%, about 30%, about 35%, about 40%, about 45%, about 50% or, about 55%, about 60%, about 70%, about 80%, or about 90% lower relative to reduced gene expression by the lipid-conjugated RNAi oligonucleotide in one or more tissues of the CNS.

In some embodiments, a reduction of target gene expression is determined by measuring a reduction in the amount or level of target mRNA, protein encoded by the target mRNA, or target gene (mRNA or protein) activity in a cell. The methods include those described herein and known to one of ordinary skill in the art.

Methods provided herein are useful in any appropriate cell type. In some embodiments, a cell is any cell that expresses the neuronal target mRNA. In some embodiments, the cell is a primary neuron cell obtained from a subject. In some embodiments, the primary cell has undergone a limited number of passages such that the cell substantially maintains is natural phenotypic properties. In some embodiments, a cell to which the oligonucleotide is delivered is ex vivo or in vitro (i.e., can be delivered to a cell in culture or to an organism in which the cell resides).

In some embodiments, the lipid-conjugated RNAi oligonucleotides disclosed herein are delivered to a cell or population of cells (e.g., neurons) using a nucleic acid delivery method known in the art including, but not limited to, injection of a solution or pharmaceutical composition containing the lipid-conjugated RNAi oligonucleotide, bombardment by particles covered by the lipid-conjugated RNAi oligonucleotide, exposing the cell or population of cells to a solution containing the lipid-conjugated RNAi oligonucleotide, or electroporation of cell membranes in the presence of the lipid-conjugated RNAi oligonucleotide. Other methods known in the art for delivering oligonucleotides to cells may be used, such as lipid-mediated carrier transport, chemical-mediated transport, and cationic liposome transfection such as calcium phosphate, and others.

In some embodiments, reduction of target gene expression is determined by an assay or technique that evaluates one or more molecules, properties or characteristics of a cell or population of cells associated with target gene expression, or by an assay or technique that evaluates molecules that are directly indicative of target gene expression in a cell or population of cells (e.g., target mRNA or protein). In some embodiments, the extent to which a lipid-conjugated RNAi oligonucleotide provided herein reduces target gene expression in a neuron is evaluated by comparing target gene expression in a neuron or population of neurons contacted with the lipid-conjugated RNAi oligonucleotide to a control cell or population of cells (e.g., a neuron or population of neurons not contacted with the lipid-conjugated RNAi oligonucleotide or contacted with a control lipid-conjugated RNAi oligonucleotide). In some embodiments, a control amount or level of target gene expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering a lipid-conjugated RNAi oligonucleotide described herein to a neuron or a population of neurons results in a reduction in expression of a neuronal target gene. In some embodiments, the reduction in target gene expression is relative to a control amount or level of target gene expression in cell or population of cells not contacted with the lipid-conjugated RNAi oligonucleotide or contacted with a control lipid-conjugated RNAi oligonucleotide. In some embodiments, the reduction in target gene expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of target gene expression. In some embodiments, the control amount or level of target gene expression is an amount or level of target mRNA and/or protein in a cell or population of cells that has not been contacted with a lipid-conjugated RNAi oligonucleotide herein. In some embodiments, the effect of delivery of a lipid-conjugated RNAi oligonucleotide to a cell or population of cells according to a method herein is assessed after any finite period or amount of time (e.g., minutes, hours, days, weeks, months). For example, in some embodiments, target gene expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the lipid-conjugated RNAi oligonucleotide to the cell or population of cells. In some embodiments, target gene expression is determined in a cell or population of cells at least about 1 month, about 2 months, about 3 months, about 4 months, about 5 months, or about 6 months or more after contacting or delivering the lipid-conjugated RNAi oligonucleotide to the cell or population of cells.

### Tissue-Specific Regulation of Gene Expression

In some embodiments, the disclosure provides a method for contacting or delivering to a cell or a population of cells a lipid-conjugated RNAi oligonucleotide described herein, wherein the cell or the population of cells is present in one or more target tissues in a subject. In some embodiments, the method comprises administering a lipid-conjugated RNAi oligonucleotide described herein to the subject, wherein the conjugate is distributed to one or more target tissues of the subject, and wherein the conjugate is contacted or delivered a cell or a population of cells within the one or more target tissues.

As used herein, a “target tissue” refers to a tissue of a subject wherein reduced expression of a target gene by a cell or a population of cells within the tissue provides one or more desirable physiological outcomes. In some embodiments, the target gene has abnormal expression in a cell or a population of cells within the one or more target tissues, wherein the abnormal expression contributes to the pathology of a disease or disorder in the subject. In some embodiments, reduced expression of the target gene by a cell or a population of cells within the target tissue functions to treat, mitigate, prevent, or alleviate the disease or disorder in the subject.

Although the distribution and/or function of a lipid-conjugated RNAi oligonucleotide within a target tissue is desirable for reducing target gene expression within a cell or population of cells that reside within the target tissue, the distribution and/or function of the conjugate to a non-target tissue has the potential to cause deleterious effects. For example, distribution of the conjugate to a non-target tissue may limit its availability for distribution to a target tissue, which in turn limits the potency and/or activity of the conjugate for reducing target gene expression within a cell or population of cells that resides within the target tissue. As another example, while the target tissue may have aberrant expression of the target gene and would benefit from reduced target gene expression to restore normal physiology, the non-target tissue may require expression of the target gene for normal physiological function. Under such circumstances, the distribution and/or function of the conjugate within the non-target tissue will impair the expression of the target gene in a manner that results in an undesirable or deleterious pathology. Accordingly, for numerous in vivo therapeutic contexts, it is beneficial to distribute the lipid-conjugated RNAi oligonucleotide to a target tissues in the subject, while limiting its distribution and/or function within one or more non-target tissues (e.g., the liver) in the subject.

In some embodiments, the disclosure provides a method for reducing or inhibiting expression of a target gene in a population of cells associated with one or more target tissues in a subject, comprising administering a lipid-conjugated RNAi oligonucleotide described herein, or a pharmaceutical composition thereof. In some embodiments, the method comprises distribution of the RNAi oligonucleotide conjugate to one or more target tissues of a subject, with minimal distribution to one or more non-target tissues of the subject. In some embodiments, the lipid-conjugated RNAi oligonucleotide is contacted or delivered to a cell or population of cells present in the one or more target tissues of the subject, with minimal contacting or delivery to a cell or population of cells present in one or more non-target tissues of the subject. In some embodiments, the expression of the target gene is reduced in the one or more target tissues without being reduced to the same or similar level in one or more non-target tissues.

In some embodiments, the method results in (i) a reduced expression of the target gene by a cell or population of cells in one or more target tissues relative to a control expression of the target gene; and (ii) a substantially equivalent expression of the target gene by a cell or population of cells in one or more non-target tissues relative to a control expression of the target gene. In some embodiments, the control expression of the target gene corresponds to the amount or level of expression of the target gene in a cell or population of cells from an equivalent tissue that is not contacted with the lipid-conjugated RNAi oligonucleotide or contacted with a control RNAi oligonucleotide conjugate. In some embodiments, the reduction of target gene expression is measured as a reduction in the amount or level of a target mRNA transcribed from the target gene or protein encoded by the target gene. In some embodiments, the method results in (i) a reduced expression of target mRNA in one or more target tissues relative to a control; and (ii) a substantially equivalent expression of target mRNA in one or more non-target tissues relative to a control. In some embodiments, the method results in (i) a reduced level of target protein in one or more target tissues relative to a control; and (ii) a substantially equivalent level of target protein in one or more non-target tissues relative to a control.

In some embodiments, the disclosure provides a method for reducing or inhibiting expression of a target gene in a population of cells associated with the CNS in a subject, comprising administering a lipid-conjugated RNAi oligonucleotide described herein, or a pharmaceutical composition thereof. In some embodiments, the method comprises distribution of the RNAi oligonucleotide conjugate to the CNS in the subject, with minimal distribution to one or more non-target tissues of the subject (e.g., the liver). In some embodiments, the lipid-conjugated RNAi oligonucleotide is contacted or delivered to a cell or population of cells present in the CNS of the subject, with minimal contacting or delivery to a cell or population of cells present in one or more non-target tissues of the subject (e.g., the liver). In some embodiments, the expression of the target gene is reduced in the CNS of the subject without being reduced to the same level in one or more non-target tissues (e.g., the liver).

In some embodiments, expression of the target gene is reduced in the CNS without being reduced to the same level in one or more non-target tissues. In some embodiments, the one or more non-target tissues comprises liver tissue. In some embodiments, the method results in (i) a reduced expression of a target gene in a cell or population of cells of the CNS relative to a control expression of the target gene; and (ii) substantially equivalent expression of the target gene in a cell or population of cells of one or more non-target tissues relative to a control expression of the target gene. In some embodiments, the control expression of the target gene corresponds to the amount or level of expression of the target gene in a cell or population of cells from an equivalent tissue that is not contacted with the lipid-conjugated RNAi oligonucleotide or contacted with a control RNAi oligonucleotide conjugate. In some embodiments, the method results in (i) a reduced expression of target gene in a cell or population of cells of the CNS relative to a control expression of the target gene (e.g., expression of the target gene in a cell or population of cells of the CNS not contacted with the lipid-conjugated RNAi oligonucleotide or contacted with a control RNAi oligonucleotide conjugate); and (ii) substantially equivalent expression of the target gene in a cell or population of cells of the liver relative to a control expression of the target gene (e.g., expression of the target gene in a cell or population of cells of the liver not contacted with the lipid-conjugated RNAi oligonucleotide or contacted with a control RNAi oligonucleotide conjugate). In some embodiments, the method results in expression of the target gene in a cell or population of cells of the CNS that is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control expression of the target gene. In some embodiments, expression of the target gene in the liver is comparable to a control expression of the target gene (e.g., having a difference not more than about ±30%, about ±25%, about ±20%, about ±15%, about ±10%, about ±5%, about ±4%, about ±3%, about ±2%, or about ±1%). In some embodiments, the reduction of target gene expression in the CNS is measured as a reduction in the amount or level of a target mRNA transcribed from the target gene or protein encoded by the target gene.

### Treatment Methods

The disclosure provides oligonucleotides for use as a medicament, in particular for use in a method for the treatment of diseases, disorders, and conditions associated with the CNS. The disclosure also provides lipid-conjugated RNAi oligonucleotide s for use, or adaptable for use, to treat a subject (e.g., a human) having a disease, disorder or condition associated with expression of a neuronal target gene that would benefit from reducing expression of the neuronal target gene. In some embodiments, the disclosure provides lipid-conjugated RNAi oligonucleotides for use, or adapted for use, to treat a subject having a disease, disorder or condition associated with neuronal target gene expression. The disclosure also provides lipid-conjugated RNAi oligonucleotides for use, or adaptable for use, in the manufacture of a medicament or pharmaceutical composition for treating a disease, disorder or condition associated with expression of a neuronal target gene. In some embodiments, the lipid-conjugated RNAi oligonucleotides for use, or adaptable for use, target mRNA and reduce expression of a neuronal target gene (e.g., via the RNAi pathway). In some embodiments, the lipid-conjugated RNAi oligonucleotides for use, or adaptable for use, target mRNA and reduce the amount or level of neuronal target mRNA, protein and/or activity.

In addition, in some embodiments of the methods herein, a subject having a disease, disorder or condition associated with expression of a neuronal target gene or is predisposed to the same is selected for treatment with a lipid-conjugated RNAi oligonucleotide herein. In some embodiments, the method comprises selecting an individual having a marker (e.g., a biomarker) for a disease, disorder or condition associated with expression of a neuronal target gene, or predisposed to the same, such as, but not limited to, target mRNA, protein, or a combination thereof. Likewise, and as detailed below, some embodiments of the methods provided by the disclosure include steps such as measuring or obtaining a baseline value for a marker of expression of a neuronal target gene, and then comparing such obtained value to one or more other baseline values or values obtained after the subject is administered the lipid-conjugated RNAi oligonucleotide to assess the effectiveness of treatment.

The disclosure also provides methods of treating a subject having, suspected of having, or at risk of developing a disease, disorder or condition associated with expression of a neuronal target gene with a lipid-conjugated RNAi oligonucleotide provided herein. In some embodiments, the disclosure provides methods of treating or attenuating the onset or progression of a disease, disorder or condition associated with expression of a neuronal target gene using the lipid-conjugated RNAi oligonucleotides provided herein. In some embodiments, the disclosure provides methods to achieve one or more therapeutic benefits in a subject having a disease, disorder or condition associated with expression of a neuronal target gene using the lipid-conjugated RNAi oligonucleotides provided herein.

In some embodiments of the methods herein, the subject is treated by administering a therapeutically effective amount of any one or more of the lipid-conjugated RNAi oligonucleotides provided herein. In some embodiments, treatment comprises reducing expression of a neuronal target gene (e.g., in the CNS). In some embodiments, the subject is treated therapeutically. In some embodiments, the subject is treated prophylactically.

In some embodiments of the methods herein, a lipid-conjugated RNAi oligonucleotide provided herein, or a pharmaceutical composition comprising the lipid-conjugated RNAi oligonucleotide, is administered to a subject having a disease, disorder or condition associated with expression of a neuronal target gene such that target gene expression is reduced in the subject, thereby treating the subject. In some embodiments, an amount or level of target mRNA is reduced in the subject. In some embodiments, an amount or level of protein encoded by the target mRNA is reduced in the subject.

In some embodiments of the methods herein, a lipid-conjugated RNAi oligonucleotide provided herein, or a pharmaceutical composition comprising the lipid-conjugated RNAi oligonucleotide, is administered to a subject having a disease, disorder or condition associated with expression of a neuronal target gene such that target gene expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression prior to administration of the lipid-conjugated RNAi oligonucleotide or pharmaceutical composition. In some embodiments, expression of a neuronal target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a subject (e.g., a reference or control subject) not receiving the lipid-conjugated RNAi oligonucleotide or pharmaceutical composition or receiving a control lipid-conjugated RNAi oligonucleotide, pharmaceutical composition or treatment.

In some embodiments of the methods herein, an lipid-conjugated RNAi oligonucleotide herein, or a pharmaceutical composition comprising the lipid-conjugated RNAi oligonucleotide, is administered to a subject having a disease, disorder or condition associated with expression of a neuronal target gene such that an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the lipid-conjugated RNAi oligonucleotide or pharmaceutical composition. In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the lipid-conjugated RNAi oligonucleotide or pharmaceutical composition or receiving a control lipid-conjugated RNAi oligonucleotide, pharmaceutical composition or treatment.

In some embodiments of the methods herein, a lipid-conjugated RNAi oligonucleotide herein, or a pharmaceutical composition comprising the lipid-conjugated RNAi oligonucleotide, is administered to a subject having a disease, disorder or condition associated with expression of a neuronal target gene such that an amount or level of protein encoded by the neuronal target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of protein encoded by the target gene prior to administration of the lipid-conjugated RNAi oligonucleotide or pharmaceutical composition. In some embodiments, an amount or level of protein encoded by a neuronal target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of protein encoded by the target gene in a subject (e.g., a reference or control subject) not receiving the lipid-conjugated RNAi oligonucleotide or pharmaceutical composition or receiving a control lipid-conjugated RNAi oligonucleotide, pharmaceutical composition or treatment.

In some embodiments of the methods herein, a lipid-conjugated RNAi oligonucleotide herein, or a pharmaceutical composition comprising the lipid-conjugated RNAi oligonucleotide, is administered to a subject having a disease, disorder or condition associated with expression of a neuronal target gene such that an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target gene activity prior to administration of the lipid-conjugated RNAi oligonucleotide or pharmaceutical composition. In some embodiments, an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target gene activity in a subject (e.g., a reference or control subject) not receiving the lipid-conjugated RNAi oligonucleotide or pharmaceutical composition or receiving a control lipid-conjugated RNAi oligonucleotide, pharmaceutical composition or treatment.

Suitable methods for determining target gene expression, an amount or level of target mRNA, an amount or level of protein encoded by the target gene, and/or an amount or level of target gene activity, in the subject, or in a sample from the subject, are known in the art. Further, the Examples set forth herein illustrate exemplary methods for determining target gene expression.

In some embodiments, target gene expression, an amount or level of target gene mRNA, an amount or level of protein encoded by a target gene, an amount or level of target gene activity, or any combination thereof, is reduced in a cell (e.g., a neuron), a population or a group of cells (e.g., an organoid), an organ (e.g., CNS), blood or a fraction thereof (e.g., plasma), a tissue (e.g., brain tissue), a sample (e.g., CSF sample or a brain biopsy sample), or any other biological material obtained or isolated from the subject. In some embodiments, expression of a neuronal target gene, an amount or level of target gene mRNA, an amount or level of protein encoded by the target gene, an amount or level of target gene activity, or any combination thereof, is reduced in more than one type of cell (e.g., neuron), more than one groups of cells, more than one organ (e.g., brain and one or more other organ(s)), more than one fraction of blood (e.g., plasma and one or more other blood fraction(s)), more than one type of tissue (e.g., brain tissue and one or more other type(s) of tissue), more than one type of sample (e.g., a brain biopsy sample and one or more other type(s) of biopsy sample) obtained or isolated from the subject.

In some embodiments, expression of a neuronal target gene is reduced in one or more of the lumbar spinal cord, lumbar dorsal root ganglion (DRG), medulla, hippocampus, somatosensory cortex, or frontal cortex. In some embodiments, expression of a neuronal target gene is reduced in one or more of the spinal cord, lumbar spinal cord, thoracic spinal cord, cervical spinal cord, lumbar dorsal root ganglion (DRG), medulla, hippocampus, somatosensory cortex, or frontal cortex. In some embodiments, expression of a neuronal target gene is reduced in the spinal cord. In some embodiments, expression of a neuronal target gene is reduced in the lumbar spinal cord. In some embodiments, expression of a neuronal target gene is reduced in the thoracic spinal cord. In some embodiments, expression of a neuronal target gene is reduced in the cervical spinal cord. In some embodiments, expression of a neuronal target gene is reduced in lumbar dorsal root Ganglion. In some embodiments, expression of a neuronal target gene is reduced in the medulla. In some embodiments, expression of a neuronal target gene is reduced in the hippocampus. In some embodiments, expression of a neuronal target gene is reduced in the somatosensory cortex. In some embodiments, expression of a neuronal target gene is reduced in the frontal cortex.

Examples of a disease, disorder or condition associated with expression of a neuronal target gene include, but are not limited to, Progressive Supranuclear Palsy (PSP), Corticobasal degeneration (CBD), Argyrophilic grain disease (AGD), Globular glial tauopathy (GGT), Ageing-related tau astrogliopathy (ARTAG), Familial Frontotemporal Dementia 17 (FTD-17), Tauopathy with Respiratory Failure, Dementia with Seizures, Pick's disease, Myotonic dystrophy 1 or 2 (MD1 or MD2), Down's syndrome, Spastic Paraplegia (SP), Niemann-Pick disease type C, Dementia with Lewy bodies (DLB), Lewy body dysphagia, Lewy body disease, Olivopontocerebellar atrophy, Striatonigral degeneration, Shy-Drager syndrome, Spinal muscular atrophy V (SMAV), Huntington's Disease (HD), Alzheimer's Disease, SCA1, SCA2, SCA3, SCA7, SCA10 (spinocerebellar ataxia type 1, 2, 3, 7 or 10), Multiple System Atrophy (MSA), Spinal and Bulbar Muscular Atrophy (SBMA, Kennedy disease), Friedrich Ataxia, Fragile X-associated tremor/ataxia syndrome (FXTAS), Fragile X syndrome (FRAXA), X-Linked Mental Retardation (XLMR), Parkinson's Disease, Dystonia, SBMA (spinobulbar muscular atrophy), neuropathic pain disorders, spinal cord injury, Dentatorubral-pallidoluysian atrophy (DRPLA), recessive CNS disorders, ALS (amyotrophic lateral sclerosis), M2DS (MECP2 duplication syndrome), FTD (frontotemporal dementia), Prion disease, Adult Onset Leukodystrophy, Alexander's Disease, Krabbe Disease, Chronic Traumatic Encephalopathy, Pelizaeus-Merzbacher disease (PMD), Lafora disease, stroke, Cerebral Amyloid Angiopathy (CAA), and Metachromatic Leukodystrophy (MLD).

In some embodiments, expression of a neuronal target gene is reduced in DRGs sufficient to treat a pain disorder associated with aberrant expression of the neuronal target gene.

In some embodiments, a disease, disorder, or condition associated with expression of a neuronal target gene is a neurodegenerative disease.

In some embodiments, the neuronal target gene may be a target gene from any mammal, such as a human. Any neuronal gene may be silenced according to the method described herein.

Methods described herein are typically involve administering to a subject a therapeutically effective amount of a lipid-conjugated RNAi oligonucleotide herein, that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g, the brain of a subject).

In some embodiments, a lipid-conjugated RNAi oligonucleotide herein, or a composition thereof, is administered intrathecally into cerebrospinal fluid (CSF) (e.g., injection or infusion into the fluid within the subarachnoid space). In some embodiments, intrathecal administration of a lipid-conjugated RNAi oligonucleotide herein, or a composition thereof, is performed as a bolus injection into the subarachnoid space. In some embodiments, intrathecal administration of a lipid-conjugated RNAi oligonucleotide herein, or a composition thereof, is performed as an infusion into the subarachnoid space. In some embodiments, intrathecal administration of a herein, or a composition thereof, is performed via a catheter into the subarachnoid space. In some embodiments, intrathecal administration of a lipid-conjugated RNAi oligonucleotide herein, or a composition thereof, is performed via a pump. In some embodiments, intrathecal administration of a lipid-conjugated RNAi oligonucleotide herein, or a composition thereof, is performed via an implantable pump. In some embodiments, administration is performed via an implantable device that operates or functions a reservoir.

In some embodiments, a lipid-conjugated RNAi oligonucleotide herein, or a composition thereof, is administered intrathecally into the cerebellomedullary cistern (also referred to as the cisterna magna). Intrathecal administration into the cisterna magna is referred to as “intracisternal administration” or “intracisternal magna (i.e.m.) administration). In some embodiments, a lipid-conjugated RNAi oligonucleotide herein, or composition thereof, is administered intrathecally into the subarachnoid space of the lumbar spinal cord. Intrathecal administration into the subarachnoid space of the lumbar spinal cord is referred to as “lumbar intrathecal (i.t.) administration”. In some embodiments, a lipid-conjugated RNAi oligonucleotide herein, or composition thereof, is administered intrathecally into the subarachnoid space of the cervical spinal cord. Intrathecal administration into the subarachnoid space of the cervical spinal cord is referred to as “cervical intrathecal (i.t.) administration”. In some embodiments, a lipid-conjugated RNAi oligonucleotide herein, or composition thereof, is administered intrathecally into the subarachnoid space of the thoracic spinal cord. Intrathecal administration into the subarachnoid space of the thoracic spinal cord is referred to as “thoracic intrathecal (i.t.) administration”. In some embodiments, a lipid-conjugated RNAi oligonucleotide herein, or composition thereof, is administered by intracerebroventricular injection or infusion into the cerebral ventricles. Intracerebroventricular administration into the ventricular space is referred to as “intracerebroventricular (i.e.v.) administration”. In some embodiments, an Ommaya reservoir is used to administer a lipid-conjugated RNAi oligonucleotide herein, or composition thereof, by intracerebroventricular injection or infusion.

In some embodiments, a lipid-conjugated RNAi oligonucleotide herein, or a composition thereof, is administered via ophthalmic, intraocular, subconjunctival, intravitreal, retrobulbar, or intracameral administration.

In some embodiments, a lipid-conjugated RNAi oligonucleotide herein, or a composition thereof, is administered via epidural administration.

In some embodiments, a lipid-conjugated RNAi oligonucleotide herein, or a composition thereof, is administered once every year, once every 6 months, once every 4 months, quarterly (once every three months), bi-monthly (once every two months), monthly or weekly. In some embodiments, a lipid-conjugated RNAi oligonucleotide herein, or a composition thereof, is administered every week or at intervals of two, or three weeks. In some embodiments, a lipid-conjugated RNAi oligonucleotide herein, or a composition thereof, is administered daily. In some embodiments, a subject is administered one or more loading doses of a lipid-conjugated RNAi oligonucleotide herein, or a composition thereof, followed by one or more maintenance doses of the lipid-conjugated RNAi oligonucleotide, or a composition thereof.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### Kits

In some embodiments, the disclosure provides a kit comprising a lipid-conjugated RNAi oligonucleotide herein, or a composition thereof, described herein, and instructions for use. In some embodiments, the kit comprises a lipid-conjugated RNAi oligonucleotide herein, or a composition thereof, described herein, and a package insert containing instructions for use of the kit and/or any component thereof. In some embodiments, the kit comprises, in a suitable container, a lipid-conjugated RNAi oligonucleotide herein, or a composition thereof, described herein, one or more controls, and various buffers, reagents, enzymes and other standard ingredients well known in the art. In some embodiments, the container comprises at least one vial, well, test tube, flask, bottle, syringe, or other container means, into which the lipid-conjugated RNAi oligonucleotide herein, or a composition thereof, is placed, and in some instances, suitably aliquoted. In some embodiments where an additional component is provided, the kit contains additional containers into which this component is placed. The kits can also include a means for containing a lipid-conjugated RNAi oligonucleotide herein, or a composition thereof, and any other reagent in close confinement for commercial sale. Such containers may include injection or blow-molded plastic containers into which the desired vials are retained. Containers and/or kits can include labeling with instructions for use and/or warnings.

In some embodiments, a kit comprises a lipid-conjugated RNAi oligonucleotide herein, or a composition thereof, described herein, and a pharmaceutically acceptable carrier, or a pharmaceutical composition comprising the lipid-conjugated RNAi oligonucleotide and instructions for treating or delaying progression of a disease, disorder or condition associated with expression of a neuronal target gene in a subject in need thereof.

### Definitions

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, exemplary methods, and materials are described herein.

General texts which describe molecular biological techniques useful herein, including the use of vectors, promoters and many other relevant topics, include Berger and Kimmel, GUIDE TO MOLECULAR CLONING TECHNIQUES, METHODS IN ENZYMOLOGY, volume 152, (Academic Press, Inc., San Diego, Calif) (“Berger”); Sambrook et al., MOLECULAR CLONING—A LABORATORY MANUAL, 2d ed., Vol. 1-3, Cold Spring Harbor Laboratory, Cold Spring Harbor, 1989 (“Sambrook”) and CURRENT PROTOCOLS IN MOLECULAR BIOLOGY, F. M. Ausubel et al., eds., CURRENT PROTOCOLS, A JOINT VENTURE BETWEEN GREENE PUBLISHING ASSOCIATES, INC. AND JOHN WILEY AND SONS, INC., (supplemented through 1999) (“Ausubel”). Examples of protocols sufficient to direct persons of skill through in vitro amplification methods, including the polymerase chain reaction (PCR), the ligase chain reaction (LCR), Q.beta.-replicase amplification and other RNA polymerase mediated techniques (e.g., NASBA), e.g., for the production of the homologous nucleic acids of the disclosure are found in Berger, Sambrook, and Ausubel, as well as in Mullis et al., (1987) U.S. Pat. No. 4,683,202; Innis et al., eds. (1990); PCR PROTOCOLS: A GUIDE TO METHODS AND APPLICATIONS (Academic Press Inc. San Diego, Calif) (“Innis”); Amheim and Levinson (Oct. 1, 1990) CandEN 36-47; J. NIH RES. (1991) 3:81-94; Kwoh et al., (1989) PROC. NATL. ACAD. SCI. USA 86: 1173; Guatelli et al (1990) PROC. NAT'L. ACAD. SCI. USA 87: 1874; Lomell et al., (1989) J. CLIN. CHEM 35: 1826; Landegren et al., (1988) SCIENCE 241: 1077-80; Van Brunt (1990) BIOTECHNOLOGY 8: 291-94; Wu and Wallace (1989) GENE 4:560; Barringer et al., (1990) GENE 89:117; and, Sooknanan and Malek (1995) BIOTECHNOLOGY 13: 563-564. Improved methods for cloning in vitro amplified nucleic acids are described in Wallace et al., U.S. Pat. No. 5,426,039. Improved methods for amplifying large nucleic acids by PCR are summarized in Cheng et al., (1994) NATURE 369: 684-85 and the references cited therein, in which PCR amplicons of up to 40 kb are generated.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one value, and/or to “about” another value. When such a range is expressed, another embodiment includes from the one value and/or to the other value. Similarly, when values are expressed as approximations, by use of the antecedent “about,” it will be understood that the value forms another embodiment. It will be further understood that the endpoints of each of the ranges are significant both in relation to the other endpoint, and independently of the other endpoint. It is also understood that there are several values disclosed herein, and that each value is also herein disclosed as “about” that value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in several different formats, and that this data, represents endpoints and starting points, and ranges for any combination of the data points. For example, if a particular datapoint “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded RNA”, “dsRNA”, or “dsRNAi” refers to an RNA oligonucleotide that is substantially in a duplex form. In some embodiments, the complementary base-pairing of duplex region(s) of a dsRNA oligonucleotide is formed between antiparallel sequences of nucleotides of covalently separate nucleic acid strands. In some embodiments, complementary base-pairing of duplex region(s) of a dsRNA formed between antiparallel sequences of nucleotides of nucleic acid strands that are covalently linked. In some embodiments, complementary base-pairing of duplex region(s) of a dsRNA is formed from single nucleic acid strand that is folded (e.g., via a hairpin) to provide complementary antiparallel sequences of nucleotides that base pair together. In some embodiments, a dsRNA comprises two covalently separate nucleic acid strands that are fully duplexed with one another. However, in some embodiments, a dsRNA comprises two covalently separate nucleic acid strands that are partially duplexed (e.g., having overhangs at one or both ends). In some embodiments, a dsRNA comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect. As used herein, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein “neuronal mRNA” and “neuronal gene” refers to any gene, mRNA, and/or protein encoded/expressed by a gene in neurons of the central nervous system. Neurons are the main cell of the nervous system and function to transmit signals to different cells within the body.

As used herein, “nicked tetraloop structure” refers to a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “oligonucleotide” refers to a short nucleic acid (e.g., less than about 100 nucleotides in length). An oligonucleotide may be single stranded (ss) or double-stranded (ds). An oligonucleotide may or may not have duplex regions. An oligonucleotide may comprise deoxyribonucleotides, ribonucleosides, or a combination of both. In some embodiments, a double-stranded oligonucleotide comprising ribonucleotides is referred to as “dsRNA”. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA or ss siRNA. In some embodiments, a double-stranded RNA (dsRNA) is an RNAi oligonucleotide.

The terms “lipid-conjugated RNAi oligonucleotide” and “oligonucleotide-ligand conjugate” are used interchangeably and refer to an oligonucleotide comprising one or more nucleotides conjugated with one or more targeting ligands (e.g., lipid).

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a dsRNA. In some embodiments, the overhang is a 3′ or 5′ overhang on the antisense strand or sense strand of a dsRNA.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylene phosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., US Provisional Patent Application Nos. 62/383,207 (filed on 2 Sep. 2016) and 62/393,401 (filed on 12 Sep. 2016). Other modifications have been developed for the 5′ end of oligonucleotides (see, e.g., Intl. Patent Application No. WO 2011/133871; U.S. Pat. No. 8,927,513; and Prakash et al., (2015) NUCLEIC ACIDS RES. 43:2993-3011).

As used herein, “reduced expression” of a target gene refers to a decrease in the amount or level of RNA transcript (e.g., target mRNA) or protein encoded by the target gene and/or a decrease in the amount or level of activity of the gene in a cell, a population of cells, a sample, or a subject, when compared to an appropriate reference (e.g., a reference cell, population of cells, sample, or subject). For example, the act of contacting a cell with an oligonucleotide or conjugate herein (e.g., an lipid-conjugated RNAi oligonucleotide comprising an antisense strand having a nucleotide sequence that is complementary to a nucleotide sequence comprising a target mRNA) may result in a decrease in the amount or level of target mRNA, protein encoded by a target gene, and/or target gene activity (e.g., via inactivation and/or degradation of target mRNA by the RNAi pathway) when compared to a cell that is not treated with the double-stranded oligonucleotide. Similarly, and as used herein, “reducing expression” refers to an act that results in reduced expression of a target gene.

As used herein, “region of complementarity” refers to a sequence of nucleotides of a nucleic acid (e.g., a dsRNA) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base. As used herein, “RNAi oligonucleotide” refers to either (a) a dsRNA having a sense strand (passenger) and antisense strand (guide), in which the antisense strand or part of the antisense strand is used by the Argonaute 2 (Ago2) endonuclease in the cleavage of a target mRNA or (b) a ss oligonucleotide having a single antisense strand, where that antisense strand (or part of that antisense strand) is used by the Ago2 endonuclease in the cleavage of a target mRNA. As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits, and humans. In one embodiment, the subject is a human or non-human primate (NHP). Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule or “moiety” (e.g., a carbohydrate, amino sugar, cholesterol, polypeptide, or lipid) that selectively binds to a cognate molecule (e.g., a receptor) of a tissue or cell of interest and/or that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “loop”, “triloop”, or “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a loop (e.g., a tetraloop or triloop) can confer a Tₘ of at least about 50° C., at least about 55° C., at least about 56° C., at least about 58° C., at least about 60° C., at least about 65° C. or at least about 75° C. in 10 mM NaHPO₄ to a hairpin comprising a duplex of at least 2 base pairs (bp) in length. In some embodiments, a loop (e.g., a tetraloop) may stabilize a bp in an adjacent stem duplex by stacking interactions. In addition, interactions among the nucleotides in a tetraloop include, but are not limited to, non-Watson-Crick base pairing, stacking interactions, hydrogen bonding and contact interactions (Cheong et al., (1990) NATURE 346:680-82; Heus and Pardi (1991) SCIENCE 253:191-94). In some embodiments, a loop comprises or consists of 3 to 6 nucleotides and is typically 4 to 5 nucleotides. In certain embodiments, a loop comprises or consists of 3, 4, 5 or 6 nucleotides, which may or may not be modified (e.g., which may or may not be conjugated to a targeting moiety). In some embodiments, a tetraloop comprises or consists of 3 to 6 nucleotides and is typically 4 to 5 nucleotides. In some embodiments, a tetraloop comprises or consists of 3, 4, 5 or 6 nucleotides, which may or may not be modified (e.g., which may or may not be conjugated to a targeting moiety). In one embodiment, a tetraloop consists of 4 nucleotides. Any nucleotide may be used in the tetraloop and standard IUPAC-IUB symbols for such nucleotides may be used as described in Cornish-Bowden ((1985) NUCLEIC ACIDS RES. 13:3021-3030). For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al., (1990) PROC. NATL. ACAD. SCI. USA 87:8467-71; Antao et al., (1991) NUCLEIC ACIDS RES. 19:5901-05). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). (See, e.g., Nakano et al., (2002) BIOCHEM. 41:4281-92; Shinji et al., (2000) NIPPON KAGAKKAI KOEN YOKOSHU 78:731). In some embodiments, the tetraloop is contained within a nicked tetraloop structure.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### EXAMPLES

### Example 1: General Methods of Preparation of Double-Stranded RNAi Oligonucleotides

### Oligonucleotide Synthesis and Purification

The double-stranded RNAi (dsRNAi) oligonucleotides described in the foregoing Examples are chemically synthesized using methods described herein. Generally, dsRNAi oligonucleotides are synthesized using solid phase oligonucleotide synthesis methods as described for 19-23mer RNAi oligonucleotides (see, e.g., Scaringe et al. (1990) NUCLEIC ACIDS RES. 18:5433-41 and Usman et al. (1987) J. AM. CHEM. SOC. 109:7845-7845; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis methodologies (see, e.g. Hughes and Ellington (2017) COLD SPRING HARB PERSPECT BIOL. 9(1):a023812; Beaucage S. L., Caruthers M. H. STUDIES ON NUCLEOTIDE CHEMISTRY V: Deoxynucleoside Phosphoramidites—A New Class of Key Intermediates for Deoxypolynucleotide Synthesis. TETRAHEDRON LETT. 1981; 22:1859-62. doi: 10.1016/S0040-4039(01)90461-7; U.S. Provisional Application No. 63/142,877 and PCT application No. PCT/US2021/42469 (each of which are incorporated herein by this reference)).

Individual RNA strands were synthesized and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-2684) and the phosphoramidite synthesis as shown below:

### Synthesis of 2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)ox)methoxy)ethoxy) ethan-1-ammonium formate (1-6)

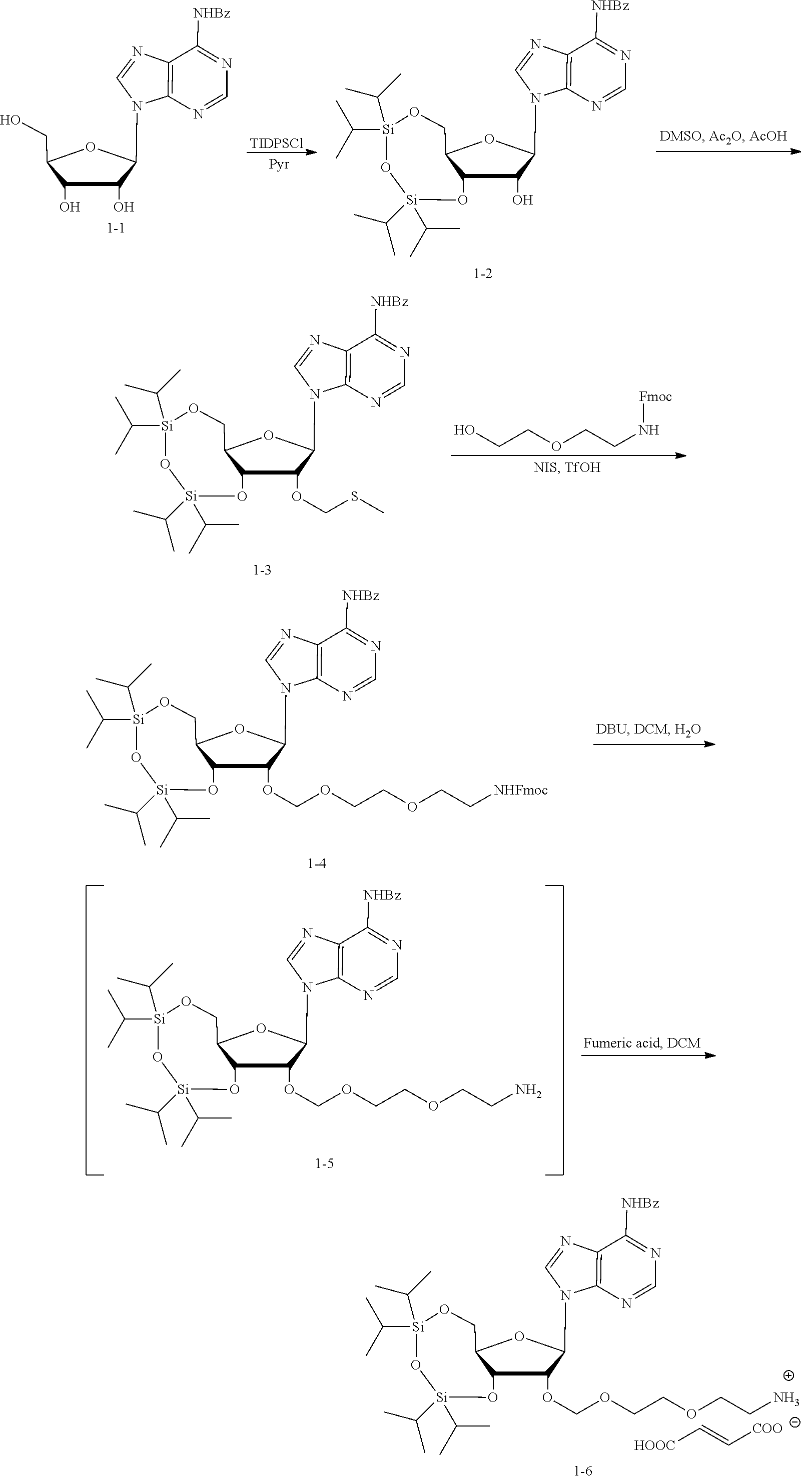

A solution of compound 1-1 (25.00 g, 67.38 mmol) in 20 mL of DMF was treated with pyridine (11 mL, 134.67 mmol) and tetraisopropyldisiloxane dichloride (22.63 mL, 70.75 mmol) at 10° C. The resulting mixture was stirred at 25° C. for 3 h and quenched with 20% citric acid (50 mL). The aqueous layer was extracted with EtOAc (3×50 mL) and the combined organic layers were concentrated in vacuo. The crude residue was recrystallized from a mixture of MTBE and n-heptane (1:15, 320 mL) to afford compound 1-2 (37.20 g, 90%) as a white oily solid.

A solution of compound 1-2 (37.00 g, 60.33 mmol) in 20 mL of DMSO was treated with AcOH (20 mL, 317.20 mmol) and Ac₂O (15 mL, 156.68 mmol). The mixture was stirred at 25° C. for 15 h. The reaction was diluted with EtOAc (100 mL) and quenched with sat. K₂CO₃ (50 mL). The aqueous layer was extracted with EtOAc (3×50 mL). The combined organic layers were concentrated and recrystallized with ACN (30 mL) to afford compound 1-3 (15.65 g, 38.4%) as a white solid.

A solution of compound 1-3 (20.00 g, 29.72 mmol) in 120 mL of DCM was treated with Fmoc-amino-ethoxy ethanol (11.67 g, 35.66 mmol) at 25° C. The mixture was stirred to afford a clear solution and then treated with 4 Å molecular sieves (20.0 g), N-iodosuccinimide (8.02 g, 35.66 mmol), and TfOH (5.25 mL, 59.44 mmol). The mixture was stirred at 30° C. until the HPLC analysis indicated >95% consumption of compound 1-3. The reaction was quenched with TEA (6 mL) and filtered. The filtrate was diluted with EtOAc, washed with sat. NaHCO₃(2×100 mL), sat. Na₂SO₃ (2×100 mL), and water (2×100 mL) and concentrated in vacuo to afford crude compound 1-4 (26.34 g, 93.9%) as a yellow solid, which was used directly for the next step without further purification.

A solution of compound 1-4 (26.34 g, 27.62 mmol) in a mixture of DCM/water (10:7, 170 mL) was treated with DBU (7.00 mL, 45.08 mmol) at 5° C. The mixture was stirred at 5-25° C. for 1 h. The organic layer was then separated, washed with water (100 mL), and diluted with DCM (130 mL). The solution was treated with fumaric acid (7.05 g, 60.76 mmol) and 4 Å molecular sieves (26.34 g) in four portions. The mixture was stirred for 1 h, concentrated, and recrystallized from a mixture of MTBE and DCM (5:1) to afford compound 1-6 (14.74 g, 62.9%) as a white solid: ¹H NMR (400 MHz, d₆-DMSO) 8.73 (s, 1H), 8.58 (s, 1H), 8.15-8.02 (m, 2H), 7.65-7.60 (m, 1H), 7.59-7.51 (m, 2H), 6.52 (s, 2H), 6.15 (s, 1H), 5.08-4.90 (m, 3H), 4.83-4.78 (m, 1H), 4.15-3.90 (m, 3H), 3.79-3.65 (m, 2H), 2.98-2.85 (m, 6H), 1.20-0.95 (m, 28H).

### Synthesis of (2R,3R,4R,5R)-5-(6-benzamido-9H-purin-9-yl)-2-((bis(4-methoxyphenyl)(phenyl)methoxy)methyl)-4-((2-(2-[lipid]-amidoethoxy)ethoxy)methoxy) tetrahydrofuran-3-yl (2-cyanoethyl) diisopropylphosphoramidite (2-4a to 2-4e)

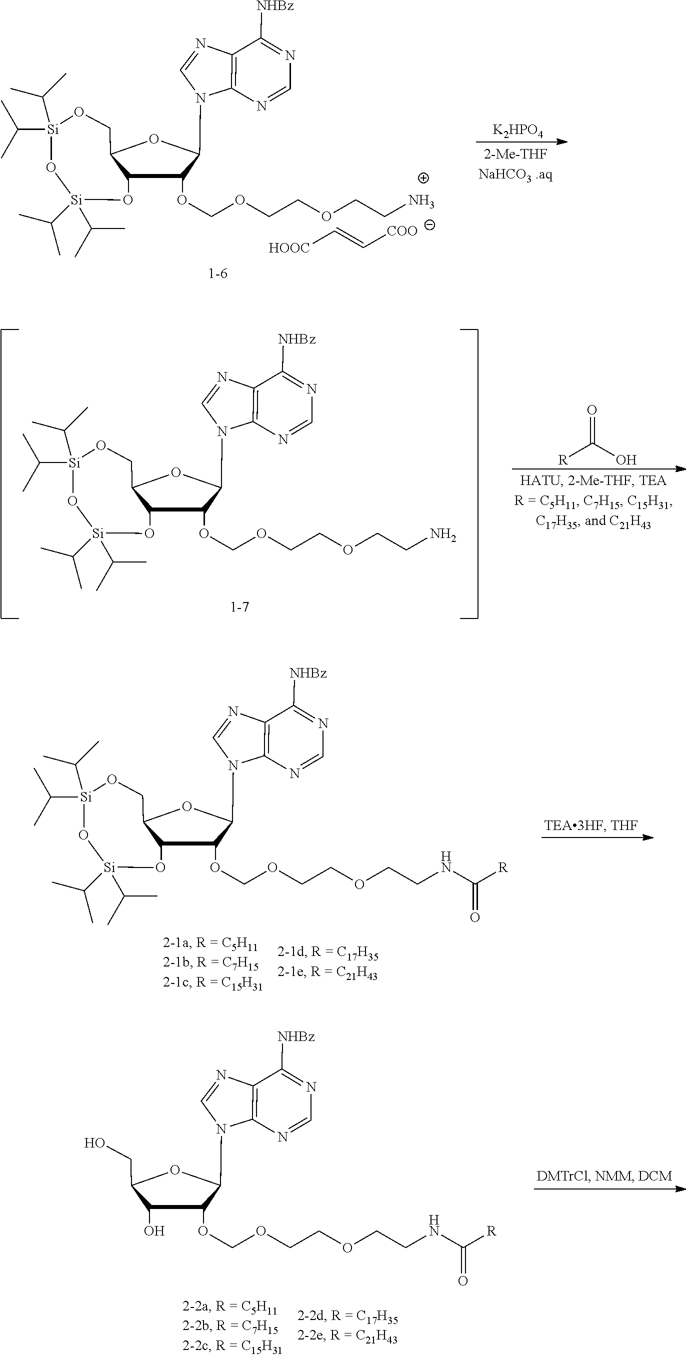

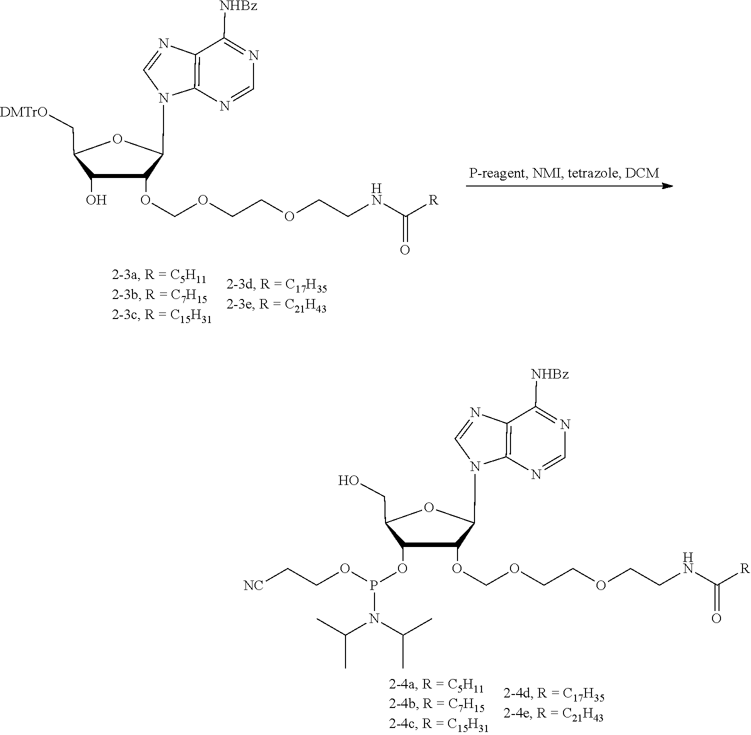

A solution of compound 1-6 (50.00 g, 59.01 mmol) in 150 mL of 2-methyltetrahydrofuran was washed with ice cold aqueous K₂HPO₄ (6%, 100 mL) and brine (20%, 2×100 mL). The organic layer was separated and treated with hexanoic acid (10.33 mL, 82.61 mmol), HATU (33.66 g, 88.52 mmol), and DMAP (10.81 g, 147.52 mmol) at 0° C. The resulting mixture was warmed to 25° C. and stirred for 1 h. The solution was washed with water (2×100 mL), brine (100 mL), and concentrated in vacuo to afford a crude residue. Flash chromatography on silica gel (1:1 hexanes/acetone) gave compound 2-1a (34.95 g, 71.5%) as a white solid.

A mixture of compound 2-1a (34.95 g, 42.19 mmol) and TEA (9.28 mL, 126.58 mmol) in 80 mL of THF was treated with triethylamine trihydrofluoride (20.61 mL, 126.58 mmol) dropwise at 10° C. The mixture was warmed to 25° C. and stirred for 2 h. The reaction was concentrated, dissolved in DCM (100 mL), and washed with sat. NaHCO₃(5×20 mL) and brine (50 mL). The organic layer was concentrated in vacuo to afford crude compound 2-2a (24.72 g, 99%), which was used directly for the next step without further purification.

A solution of compound 2-2a (24.72 g, 42.18 mmol) in 50 mL of DCM was treated with N-methylmorpholine (18.54 mL, 168.67 mmol) and DMTr-Cl (15.69 g, 46.38 mmol). The mixture was stirred at 25° C. for 2 h and quenched with sat. NaHCO₃(50 mL). The organic layer was separated, washed with water, concentrated to afford a slurry crude. Flash chromatography on silica gel (1:1 hexanes/acetone) gave compound 2-3a (30.05 g, 33.8 mmol, 79.9%) as a white solid.

A solution of compound 2-3a (25.00 g, 28.17 mmol) in 50 mL of DCM was treated with N-methylmorpholine (3.10 mL, 28.17 mmol) and tetrazole (0.67 mL, 14.09 mmol) under nitrogen atmosphere. Bis(diisopropylamino) chlorophosphine (9.02 g, 33.80 mmol) was added to the solution dropwise and the resulting mixture was stirred at 25° C. for 4 h. The reaction was quenched with water (15 mL), and the aqueous layer was extracted with DCM (3×50 mL). The combined organic layers were washed with sat. NaHCO₃(50 mL), concentrated to afford a crude solid that was recrystallized from a mixture of DCM/MTBE/n-hexane (1:4:40) to afford compound 2-4a (25.52 g, 83.4%) as a white solid: ¹H NMR (400 MHz, d₆-DMSO) 11.25 (s, 1H), 8.65-8.60 (m, 2H), 8.09-8.02 (m, 2H), 7.71 (s, 1H), 7.67-7.60 (m, 1H), 7.59-7.51 (m, 2H), 7.38-7.34 (m, 2H), 7.30-7.25 (m, 7H), 6.85-6.79 (m, 4H), 6.23-6.20 (m, 1H), 5.23-5.14 (m, 1H), 4.80-4.69 (m, 3H), 4.33-4.23 (m, 2H), 3.90-3.78 (m, 1H), 3.75 (s, 6H), 3.74-3.52 (m, 3H), 3.50-3.20 (m, 6H), 3.14-3.09 (m, 2H), 3.09 (s, 1H), 2.82-2.80 (m, 1H), 2.65-2.60 (m, 1H), 2.05-1.96 (m, 2H), 1.50-1.39 (m, 2H), 1.31-1.10 (m, 14H), 1.08-1.05 (m, 2H), 0.85-0.79 (m, 3H); ³¹P NMR (162 MHz, d₆-DMSO) 149.43, 149.18.

Compound 2-4b, 2-4c, 2-4d, and 2-4e were prepared using similar procedures described above for compound 2-4a. Compound 2-4b was obtained (25.50 g, 85.4%) as a white solid: ¹H NMR (400 MHz, d₆-DMSO) 11.23 (s, 1H), 8.65-8.60 (m, 2H), 8.05-8.02 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.97 (m, 2H), 1.50-1.38 (m, 2H), 1.31-1.10 (m, 18H), 1.08-1.05 (m, 2H), 0.85-0.78 (m, 3H); ³¹P NMR (162 MHz, d₆-DMSO) 149.43, 149.19.

Compound 2-4c was obtained (36.60 g, 66.3%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.25-5.17 (m, 1H), 4.80-4.69 (m, 3H), 4.40-4.21 (m, 2H), 3.91-3.80 (m, 1H), 3.74 (s, 6H), 3.74-3.50 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.33-1.12 (m, 38H), 1.08-1.05 (m, 2H), 0.86-0.80 (m, 3H); ³¹P NMR (162 MHz, d₆-DMSO) 149.42, 149.17.

Compound 2-4d was obtained (26.60 g, 72.9%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.33 (m, 2H), 7.30-7.25 (m, 7H), 6.89-6.80 (m, 4H), 6.21-6.15 (m, 1H), 5.22-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.08 (m, 38H), 1.08-1.05 (m, 2H), 0.85-0.79 (m, 3H); ³¹P NMR (162 MHz, d₆-DMSO) 149.47, 149.22.

Compound 2-4e was obtained (38.10 g, 54.0%) as a white solid: ¹H NMR (400 MHz, d₆-DMSO) 11.21 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.73 (s, 6H), 3.74-3.52 (m, 3H), 3.47-3.22 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.06 (m, 46H), 1.08-1.06 (m, 2H), 0.85-0.77 (m, 3H); ³¹P NMR (162 MHz, d₆-DMSO) 149.41, 149.15.

The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide was injected into a capillary, run in an electric field of 444 V/cm, and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DE™ Biospectometry Work Station (Applied Biosystems; Foster City, CA) following the manufacturer's recommended protocol. Relative molecular masses of all oligomers were obtained, often within 0.2% of expected molecular mass.

### Preparation of Duplexes

Single strand RNA oligomers were resuspended (e.g., at 100 μM concentration) in duplex buffer consisting of 100 mM potassium acetate, 30 mM HEPES, pH 7.5. Complementary sense and antisense strands were mixed in equal molar amounts to yield a final solution of, for example, 50 μM duplex. Samples were heated to 100° C. for 5′ in RNA buffer (IDT) and were allowed to cool to room temperature before use. The dsRNA oligonucleotides were stored at −20° C. Single strand RNA oligomers were stored lyophilized or in nuclease-free water at −80° C.

The synthesis methods described herein are used to generate the lipid-conjugated oligonucleotides described in Example 3.

### Example 2: Synthesis of Lipid-Conjugated Oligonucleotides

The following schematic depicts the synthesis of a blunt end oligonucleotide with a C16-lipid at the 5′-end. Lipid-conjugated blunt-ended oligonucleotides described herein can be synthesized using post-synthetic methods described in detail in U.S. Provisional Application No. 63/142,877 and PCT application No. PCT/US2021/42469. Specifically, the oligonucleotides can be synthesized using a post-synthetic conjugation approach such as that depicted below. In Eppendorf tube 1, a solution of palmitic acid in DMA was treated with HATU at rt. In Eppendorf tube 2, a solution of oligo sense strand in H₂O was treated with DIPEA. The solution in Eppendorf tube 1 was added to the Eppendorf tube 2 and mixed using ThermoMixer at rt. After the reaction was completed indicated by LC-MS analysis, the reaction mixture was diluted with 5 mL of water and purified by revers phase XBridge C18 column using a 5-95% gradient of 100 mM TEAA in ACN and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were then lyophilized to afford an amorphous white solid.

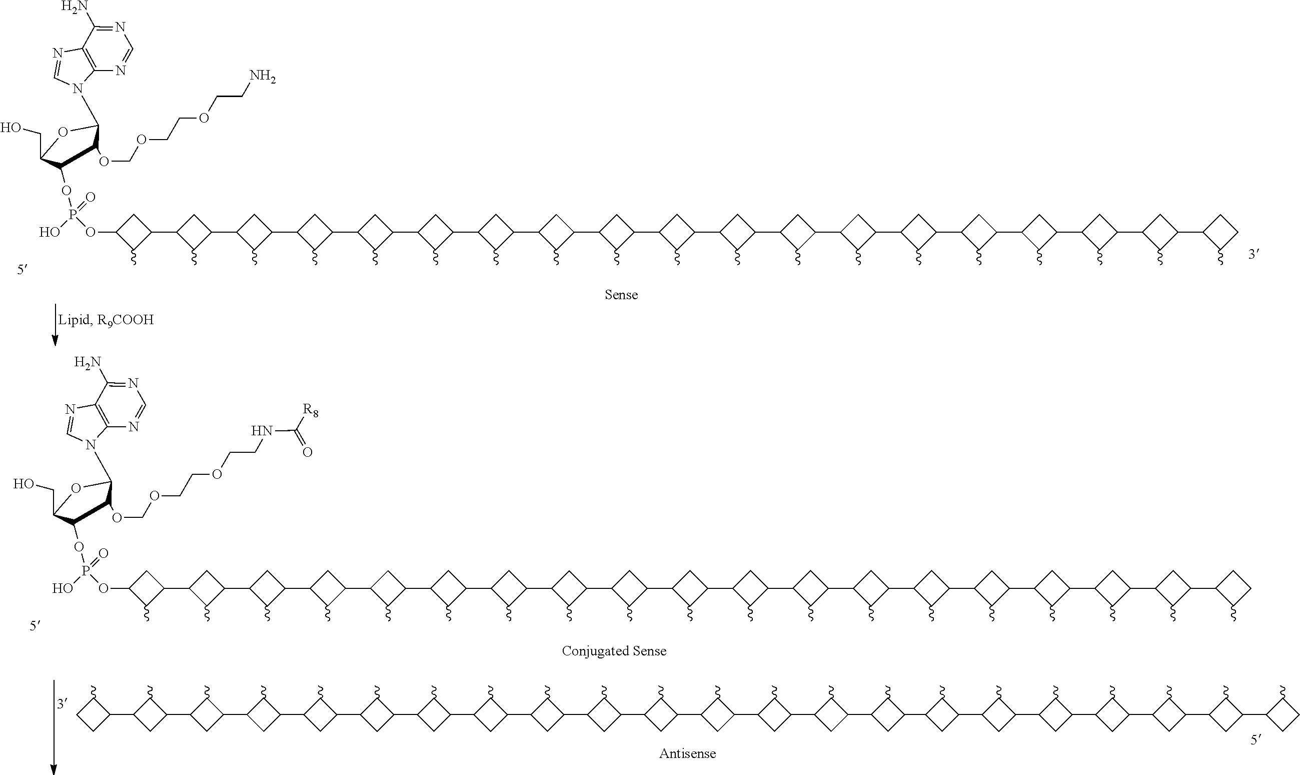

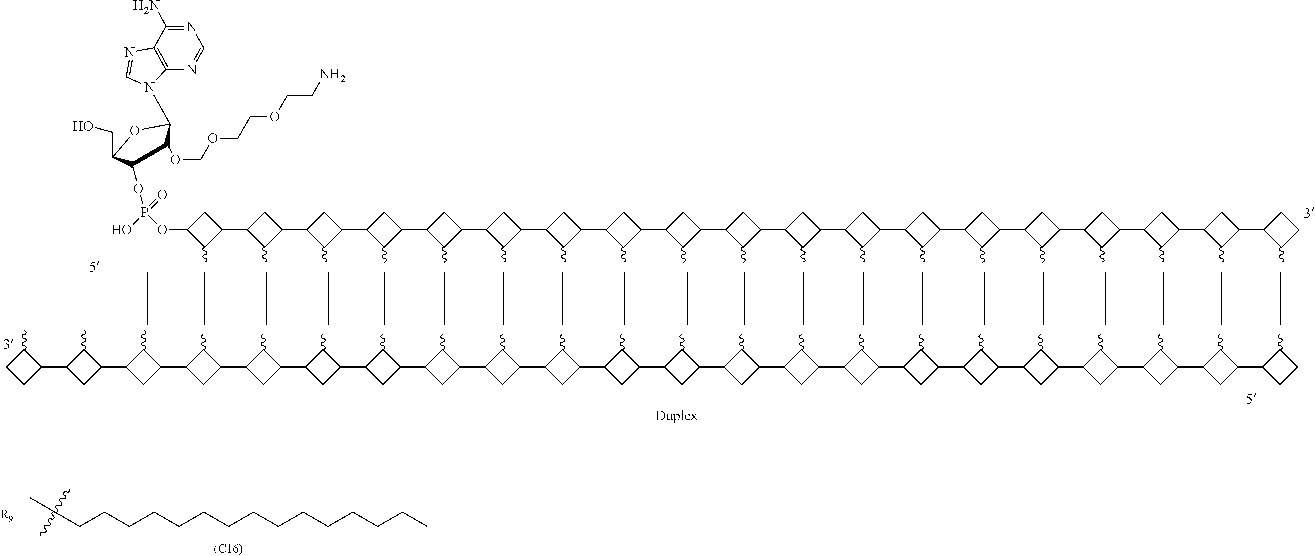

### Example 3: Lipid-Conjugated Blunt-End RNAi Oligonucleotides Reduce Neuronal Target Gene Expression in the CNS

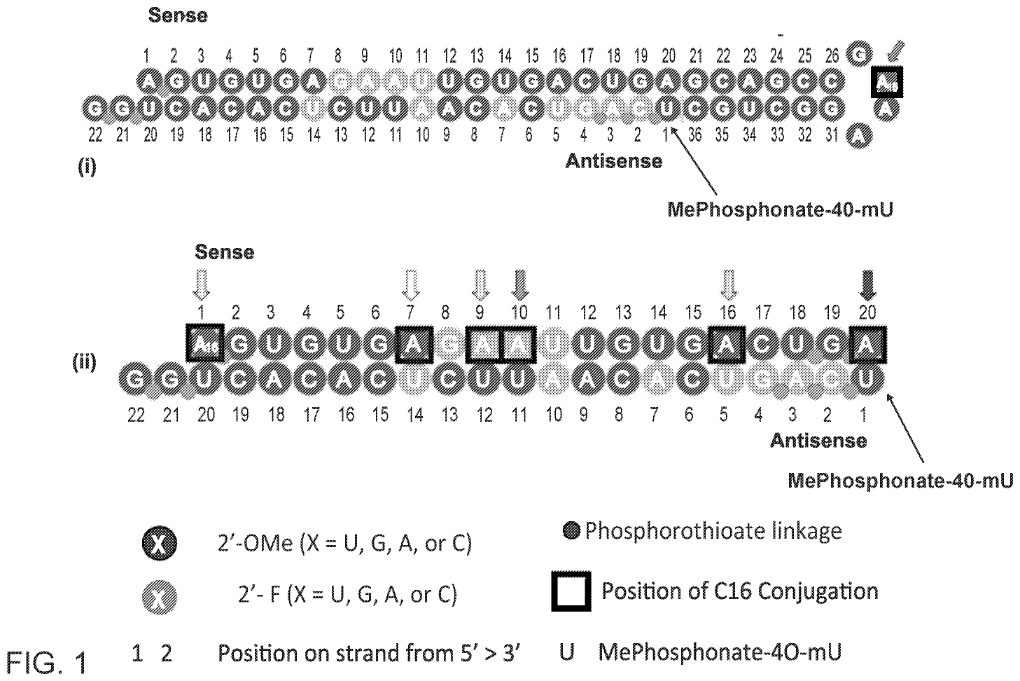

To identify a lipid-conjugated RNAi oligonucleotide capable of reducing mRNA expression with the highest selectivity in neurons of the central nervous system (CNS), a series of C16 conjugated RNAi oligonucleotides were generated by methods described in Example 1. Specifically, oligonucleotides having a blunt-end at the 3′terminus and a 2-nucleotide overhang at the 5′terminus were generated with a C16 lipid conjugated at a different position (positions 1, 7, 9, 10, 16 and 20) in the sense strand as shown in the patterns below. For comparison, an oligonucleotide having a nicked-tetraloop structure with a C16 lipid conjugated at position 28 in the stem-loop was generated as the control based on prior studies. Each oligonucleotide tested comprised an antisense strand having a region of complementarity to mRNA encoding Tubulin Beta 3 Class III (Tubb3). Tubb3 is a protein primarily expressed in neurons and is targeted herein to demonstrate delivery of lipid-conjugated RNAi oligonucleotides to neurons of the CNS. The unmodified sense and antisense strands are provided in SEQ ID NOs: 1 and 2, respectively, and the modified strands are shown in Table 1. Schematics of the oligonucleotides tested is provided in FIG. 1 Modifications are shown below:

P28 Sense Strand:
[mXs][mX][mX][mX][mX][mX][mX][fX][fX][fX][fX][mX][mX][mX][mX][mX][mX][mX]
[mX][mX][mX][mX][mX][mX][mX][mX][mX][ademX-
C16][mX][mX][mX][mX][mX][mX][mX][mX]
P1 Sense Strand:
[ademX-C16s][mX][mX][mX][mX][mX][mX][fX][fX][fX][fX][mX][mX][mX][mX][mX]
[mX][mXs][mXs][mX]
P7 Sense Strand:
[mXs][mX][mX][mX][mX][mX][ademX-C16][fX][fX][fX][fX][mX][mX][mX][mX][mX]
[mX][mXs][mXs][mX]
P9 Sense Strand:
[mXs][mX][mX][mX][mX][mX][mX][fX][ademX-C16][fX][fX][mX][mX][mX][mX][mX]
[mX][mXs][mXs][mX]
P10 Sense Strand:
[mXs][mX][mX][mX][mX][mX][mX][fX][fX][ademX-C16][fX][mX][mX][mX][mX][mX]
[mX][mXs][mXs][mX]
P16 Sense Strand:
[mXs][mX][mX][mX][mX][mX][mX][fX][fX][fX][fX][mX][mX][mX][mX][ademX-C16]
[mX][mXs][mXs][mX]
P20 Sense Strand:
[mXs][mX][mX][mX][mX][mX][mX][fX][fX][fX][fX][mX][mX][mX][mX][mX]
[mX][mXs][mXs][ademXs-C16]

Each of P28, P1, P7, P9, P10, P16, and P20 hybridized to an antisense strand having the following modification pattern:

Antisense Strand:
[MePhosphonate-4O-mXs][fXs][fXs][fX]
[fX][mX][fX][mX]mX][fX][mX][mX][mX]
[fX][[mX][mX][mX][mX][mX][mXs][mXs][mX]

### Modification Key:

| | |
|---|---|
| [mXs] | 2′-O-methyl modified nucleotide with a |
| | phosphorothioate linkage to the neighboring |
| | nucleotide |
| [fXs] | 2′- fluoro modified nucleotide with a |
| | phosphorothioate linkage to the neighboring |
| | nucleotide |
| [mX] | 2′-O-methyl modified nucleotide with |
| | phosphodiester linkages to neighboring |
| | nucleotides |
| [fX] | 2′- fluoro modified nucleotide with |
| | phosphodiester linkages to neighboring |
| | nucleotides |
| [ademX-C16] | C16 lipid attached to adenine nucleotide |
| [ademX-C16s] | C16 lipid attached to adenine nucleotide with a |
| | phosphorothioate linkage to the neighboring nucleotide |
| [MePhosphonate- | 4′-O-monomethylphosphonate-2′-O-methyl modified |
| 4O-mXs] | nucleotide |

TABLE 1
Lipid-conjugated RNAi Blunt-End oligonucleotides

| | Sense Strand | Antisense Strand |
|---|---|---|
| Oligonucleotide | SEQ ID NO | SEQ ID NO |
| P28 C16* | 3 | 10 |
| P1 C16 | 4 | 10 |
| P7 C16 | 5 | 10 |
| P9 C16 | 6 | 10 |
| P10 C16 | 7 | 10 |
| P16 C16 | 8 | 10 |
| P20 C16 | 9 | 10 |

*Indicates tetraloop oligonucleotide

To evaluate the oligonucleotides in Table 1, C57BL/6 female mice, 6-8 weeks old, were given a single intrathecal (i.t.) cerebrospinal fluid injection with 500 μg of oligonucleotide or artificial cerebrospinal fluid (aCSF). Target knockdown was assessed 7 days after injection.

RNA was extracted from tissue samples from the lumbar spinal cord, lumbar dorsal root ganglion (DRG), medulla, hippocampus, somatosensory cortex, and frontal cortex to determine murine Tubb3 mRNA levels by qPCR (normalized to endogenous housekeeping gene Rpl23, as indicated). The levels of murine Tubb3 mRNA were determined using PrimeTime™ qPCR Probe Assays (IDT). The qPCR was performed using PrimeTime™ qPCR Probe Assays, which consisted of a primer pair and fluorescently labeled 5′ nuclease probe specific to murine Tubb3 mRNA. The percentage of murine Tubb3 mRNA remaining in the samples from treated mice was determined using the 2^{-ΔΔCt} (“delta-delta Ct”) method (Livak and Schmittgen (2001) METHODS 25:402-408).

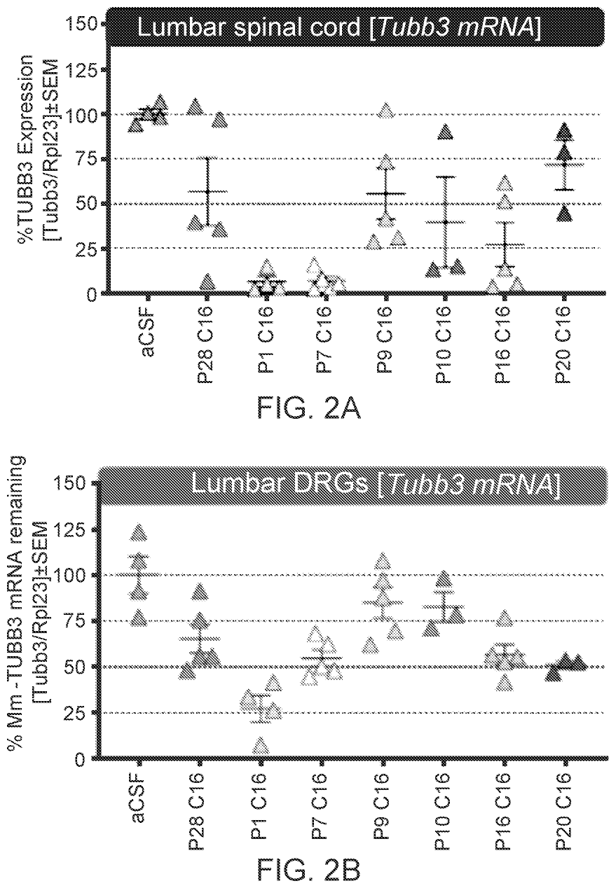

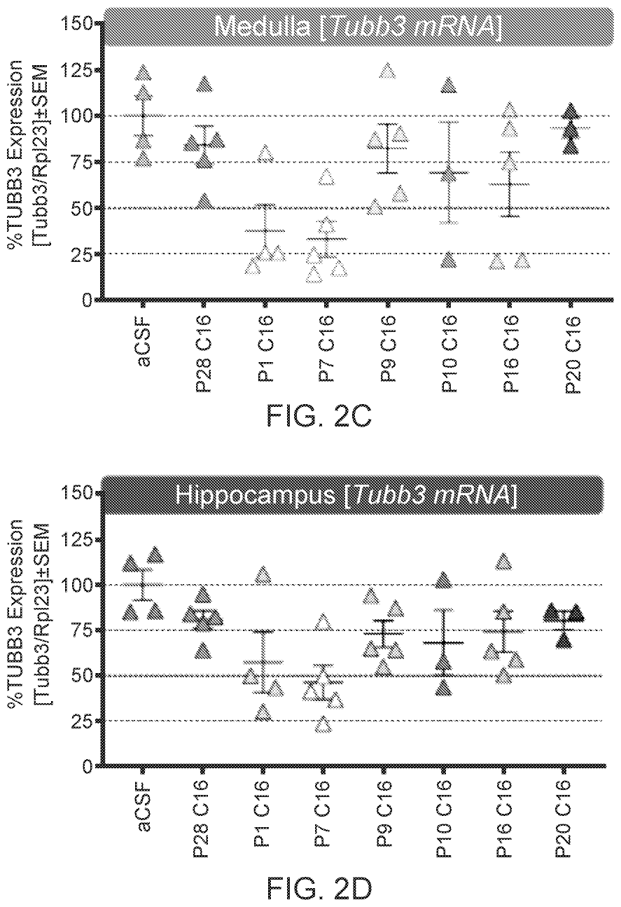

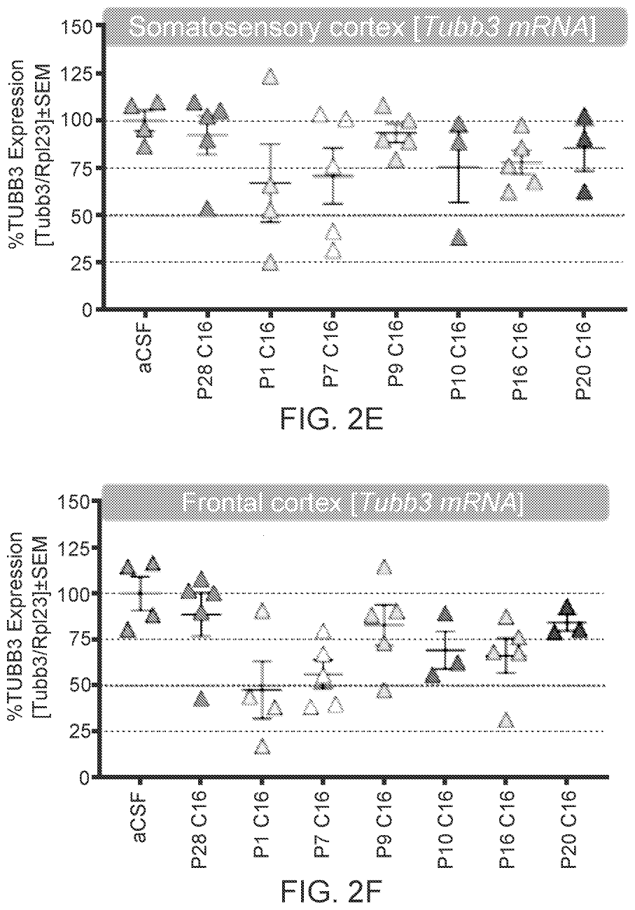

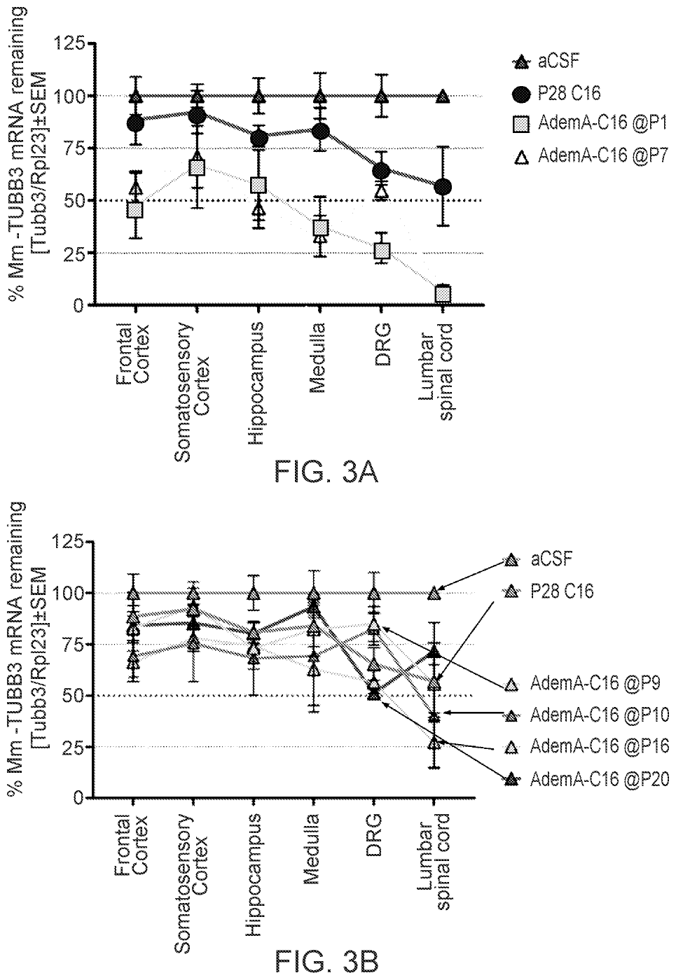

Tubb3 mRNA expression was reduced in several tissues of the CNS (FIGS. 2A-2F). Specifically, lipid conjugation at P1 or P7 showed highest levels of Tubb3 knockdown in the lumbar spine (FIG. 2A); lipid conjugation at P1 showed highest levels of Tubb3 knockdown in lumbar DRGs (FIG. 2B); and lipid conjugation at P1 or P7 showed highest levels of Tubb3 knockdown in the medulla, hippocampus, somatosensory cortex, and frontal cortex (FIGS. 2C-2F). FIGS. 3A-3B compile the data in FIGS. 2A-2F. Overall, lipid conjugation at positions 1 or 7 provide higher levels of mRNA knockdown in neurons relative to the control nicked-tetraloop oligonucleotide having a lipid conjugated at position 28, whereas lipid conjugation at positions 9, 10, 16 and 20 provide either the same level of mRNA knockdown or less than the control nicked-tetraloop oligonucleotide having a lipid conjugated at position 28.

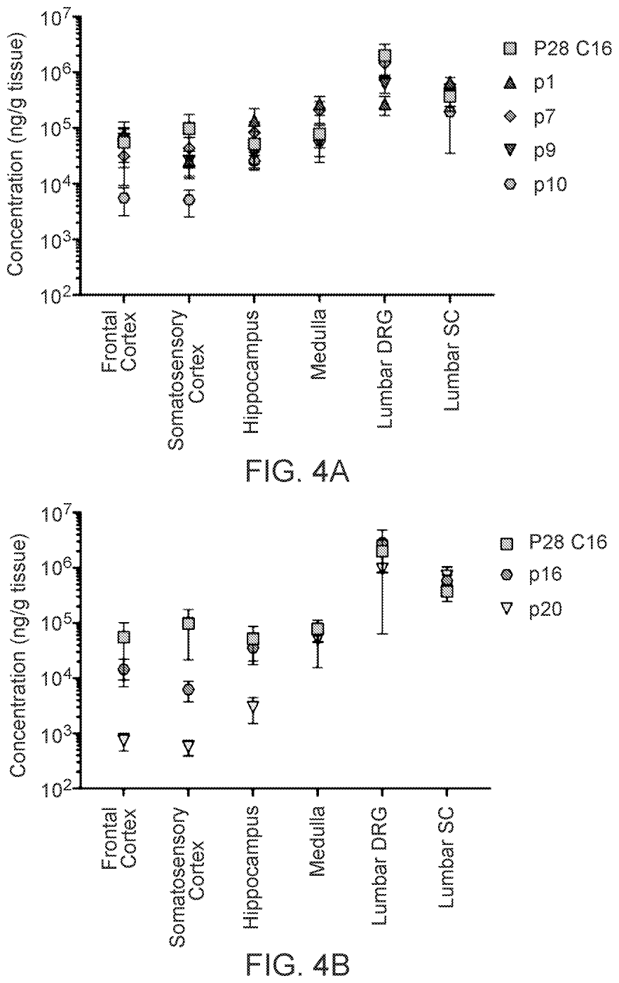

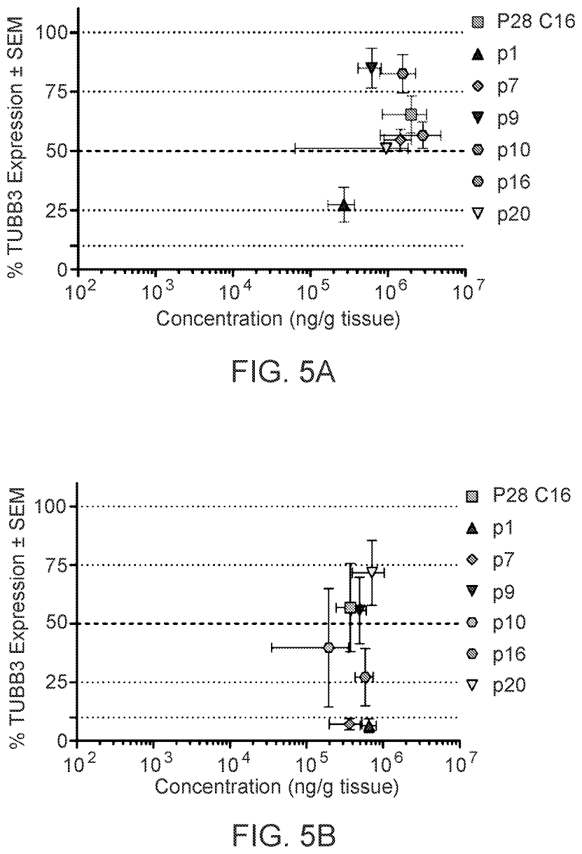

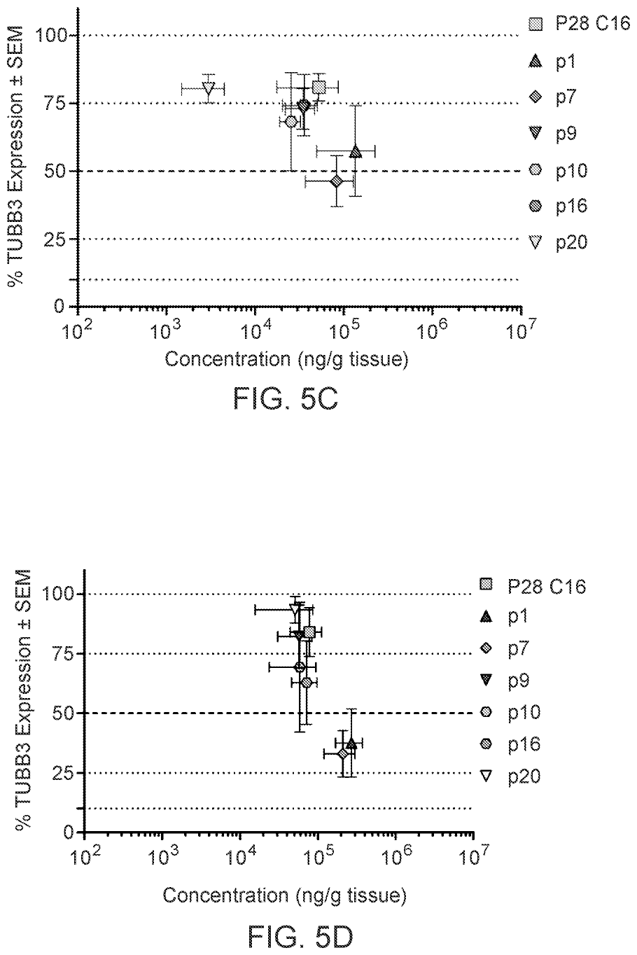

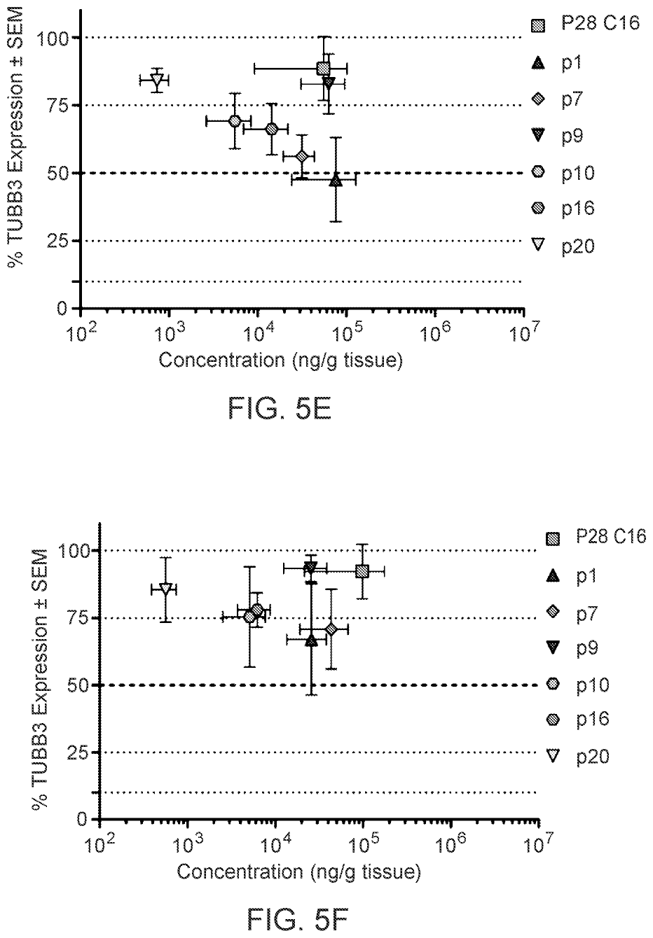

The concentration of each oligonucleotide 7-days after injection was measured in several tissues of the CNS. Blunt-ended oligonucleotides having a lipid conjugated to a nucleotide on the sense strand demonstrated similar tissue exposure to that of the control nicked-tetraloop oligonucleotide with a lipid conjugated at position 28 (FIGS. 4A-4B). However, lower levels of exposure were observed in the furthest brain regions (frontal cortex and somatosensory cortex) with the P10, P16, and P20 oligonucleotides compared to the control nicked-tetraloop oligonucleotide with a lipid conjugated at position 28. When comparing the overall knockdown efficiency (as demonstrated in FIGS. 2A-2F) to that of the tissue exposure to the lipid-conjugated RNAi oligonucleotides (as demonstrated in FIGS. 4A-4B), blunt-ended oligonucleotides with a lipid conjugated to a nucleotide of the sense strand demonstrated increased knockdown when compared to similar exposure levels of the control nicked-tetraloop oligonucleotide with a lipid conjugated at position 28 (FIGS. 5A-5F). Particularly, enhanced potency was observed in oligonucleotides having a lipid conjugated at P1 or P7 relative to the P28 tetraloop structure, as well as other positional variations of palmitic acid in the blunt-ended passenger strand. These results demonstrate that lipid-conjugated RNAi oligonucleotides exhibit the ability to reduce target gene expression in neurons found in multiple different anatomical regions of the CNS, including difficult to reach tissues such as the frontal cortex and hippocampus.

### Example 4: Lipid-Conjugated Tetraloop RNAi Oligonucleotides Reduce Neuronal Target Gene Expression in the CNS

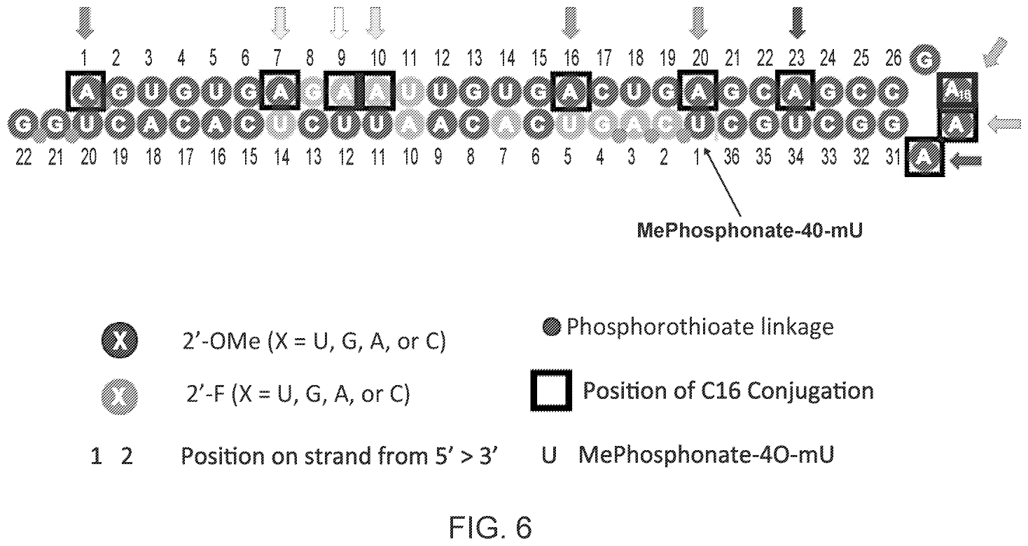

RNAi oligonucleotides comprising a tetraloop and conjugated to a lipid at various positions of the sense strand were evaluated for their ability to reduce neuronal target expression in the CNS. Tetraloop RNAi oligonucleotides conjugated to a C16 lipid were generated as described in Example 3. Specifically, a C16 lipid was conjugated at one of nucleotide positions (P) 1, 7, 9, 10, 16, 20, 23, 28, 29, and 30 of the sense strand as shown in the modification patterns below. Each oligonucleotide tested comprised an antisense strand having a region of complementarity to mRNA encoding Tubb3. The unmodified sense and antisense strands are provided in SEQ ID NOs: 20 and 2, respectively, and the modified strands are shown in Table 2. Schematics of the oligonucleotides tested is provided in FIG. 6

### Tetraloop RNAi Oligonucleotide Modifications Patterns:

P1 Sense Strand:
[ademX-C16s][mX][mX][mX][mX][mX][mX][fX][fX][fX][fX][mX][mX][mX][mX][mX]
[mX][mX][mX][mX][mX][mX][mX][mX][mX][mX][mX][mX][mX][mX][mX][mX]
[mX][mX][mX][mX]
P7 Sense Strand:
[mXs][mX][mX][mX][mX][mX][ademX-C16][fX][fX][fX][fX][mX][mX][mX][mX][mX]
[mX][mX][mX][mX][mX][mX][mX][mX][mX][mX][mX][mX][mX][mX][mX][mX]
[mX][mX][mX][mX]
P9 Sense Strand:
[mXs][mX][mX][mX][mX][mX][mX][fX][ademX-C16][fX][fX][mX][mX][mX][mX][mX]
[mX][mX][mX][mX][mX][mX][mX][mX][mX][mX][mX][mX][mX][mX][mX][mX]
[mX][mX][mX][mX]
P10 Sense Strand:
[mXs][mX][mX][mX][mX][mX][mX][fX][fX][ademX-C16][fX][mX][mX][mX][mX][mX]
[mX][mX][mX][mX][mX][mX][mX][mX][mX][mX][mX][mX][mX][mX][mX][mX]
[mX][mX][mX][mX]
P16 Sense Strand:
[mXs][mX][mX][mX][mX][mX][mX][fX][fX][fX][fX][mX][mX][mX][mX][ademX-C16]
[mX][mX][mX][mX][mX][mX][mX][mX][mX][mX][mX][mX][mX][mX][mX][mX]
[mX][mX][mX][mX]
P20 Sense Strand:
[mXs][mX][mX][mX][mX][mX][mX][fX][fX][fX][fX][mX][mX][mX][mX][mX]
[mX][mX][mX][ademX-C16][mX][mX][mX][mX][mX][mX][mX][mX][mX][mX]
[mX][mX][mX][mX][mX][mX]
P23 Sense Strand:
[mXs][mX][mX][mX][mX][mX][mX][fX][fX][fX][fX][mX][mX][mX][mX][mX]
[mX][mX][mX][mX][mX][mX][ademX-
C16][mX][mX][mX][mX][mX][mX][mX][mX][mX][mX][mX][mX][mX]
P28 Sense Strand:
[mXs][mX][mX][mX][mX][mX][mX][fX][fX][fX][fX][mX][mX][mX][mX][mX][mX][mX]
[mX][mX][mX][mX][mX][mX][mX][mX][mX][ademX-
C16][mX][mX][mX][mX][mX][mX][mX][mX]
P29 Sense Strand:
[mXs][mX][mX][mX][mX][mX][mX][fX][fX][fX][fX][mX][mX][mX][mX][mX][mX][mX]
[mX][mX][mX][mX][mX][mX][mX][mX][mX][X][ademX-
C16][mX][mX][mX][mX][mX][mX][mX]
P30 Sense Strand:
[mXs][mX][mX][mX][mX][mX][mX][fX][fX][fX][fX][mX][mX][mX][mX][mX][mX][mX]
[mX][mX][mX][mX][mX][mX][mX][mX][mX][mX][mX][ademX-
C16][mX][mX][mX][mX][mX][mX]

Each of P1, P7, P9, P10, P16, P20, P23, P28, P29, and P30 hybridized to an antisense strand having the following modification pattern:

Antisense Strand:
[MePhosphonate-4O-mXs][fXs][fXs][fX][fX][mX]
[fX][mX][mX][fX][mX][mX][mX][fX][mX][mX][mX]
[mX][mX][mXs][mXs][mX]

### Modification Key: Provided in Example 3

TABLE 2
Lipid-conjugated RNAi oligonucleotides with stem loop

| | Sense Strand | Antisense Strand |
|---|---|---|
| Oligonucleotide | SEQ ID NO | SEQ ID NO |
| P1 C16 | 11 | 10 |
| P7 C16 | 12 | 10 |
| P9 C16 | 13 | 10 |
| P10 C16 | 14 | 10 |
| P16 C16 | 15 | 10 |
| P20 C16 | 16 | 10 |
| P23 C16 | 17 | 10 |
| P28 C16 | 3 | 10 |
| P29 C16 | 18 | 10 |
| P30 C16 | 19 | 10 |

To evaluate the tetraloop RNAi oligonucleotide-lipid conjugates in Table 2, C57BL/6 female mice, 6-8 weeks old, were treated with 500 μg of lipid-conjugated tetraloop RNAi oligonucleotide formulated in artificial cerebrospinal fluid (aCSF) via intrathecal (i.t.) lumbar injection. Control animals were injected with aCSF only. Target knockdown was assessed 7 days after injection.

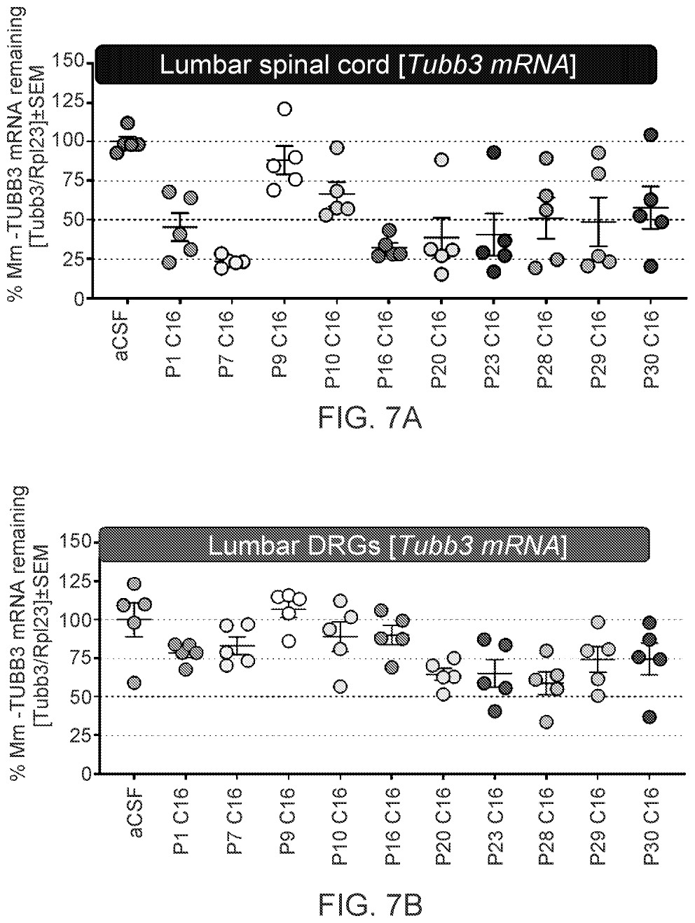

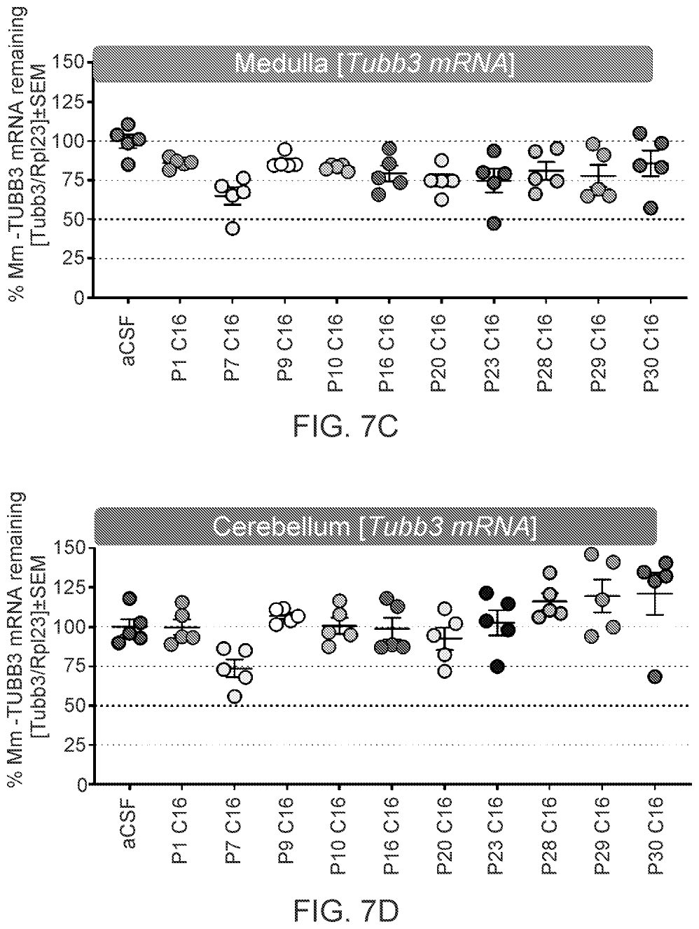

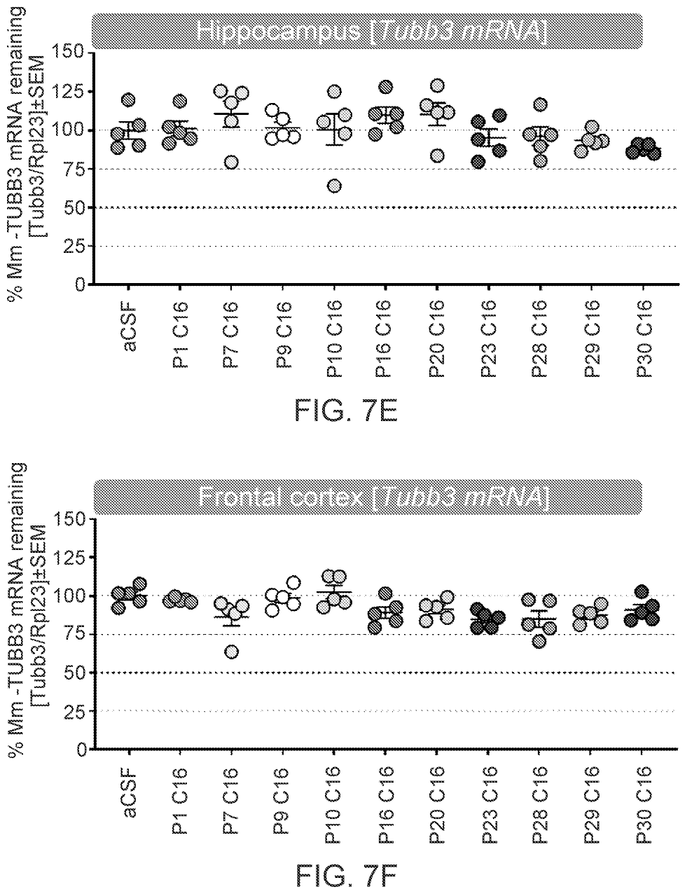

RNA was extracted from tissue samples from the lumbar spinal cord, lumbar dorsal root ganglion (DRG), medulla, cerebellum, hippocampus, and frontal cortex to determine murine Tubb3 mRNA levels by qPCR as described in Example 3. Tubb3 mRNA expression was reduced by about 50% or greater in samples from the lumbar spinal cord 7 days following injection of tetraloop RNAi oligonucleotides with a C16 lipid conjugated at position P1, P7, P16, P20, P23, P28, P29, or P30 (FIG. 7A). Less reduction in Tubb3 mRNA expression was observed in other regions of the CNS (FIGS. 7B-7F). These results demonstrate that lipid-conjugated RNAi oligonucleotides comprising a tetraloop exhibit the ability to reduce target (e.g., Tubb3) gene expression in CNS neurons. Further, these results suggest that the ability of lipid-conjugated tetraloop RNAi oligonucleotides to reduce target gene expression in neurons may be proximal, localized, and/or restricted to a region of the CNS at or near the site of administration following intrathecal (i.t.) lumbar injection.

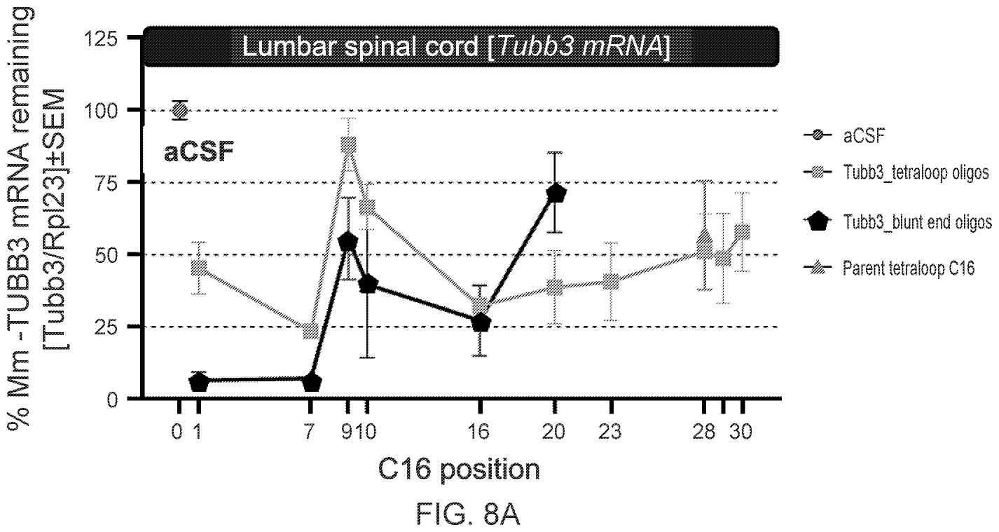

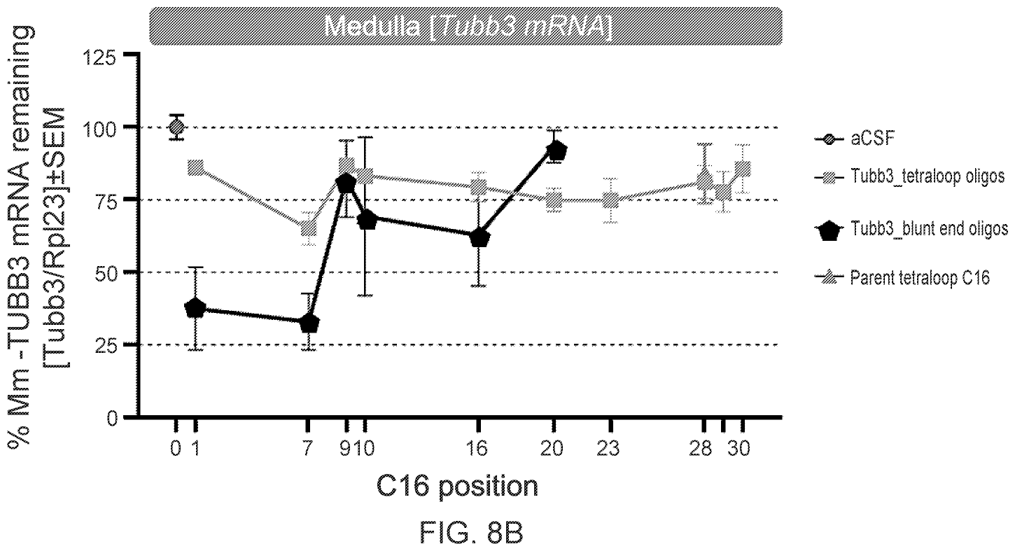

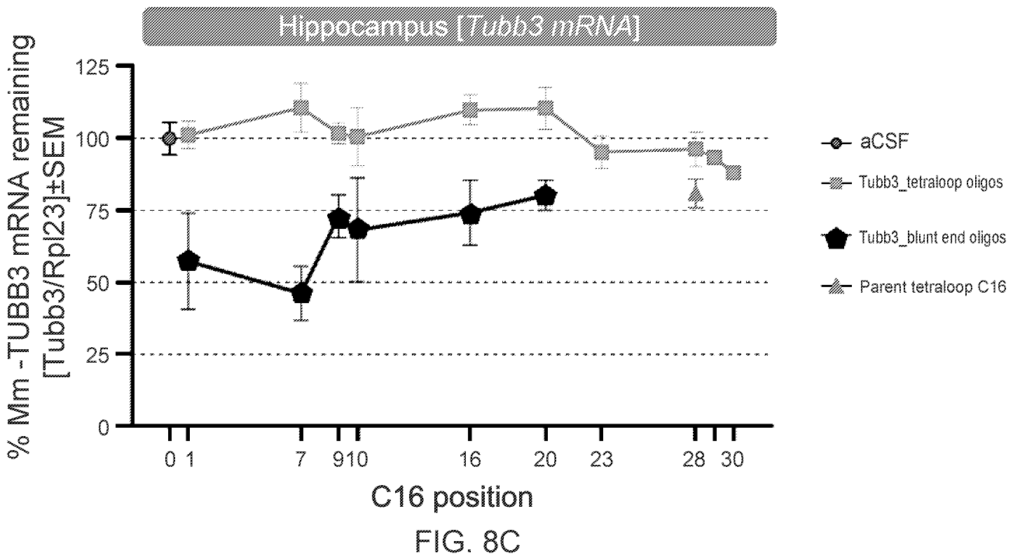

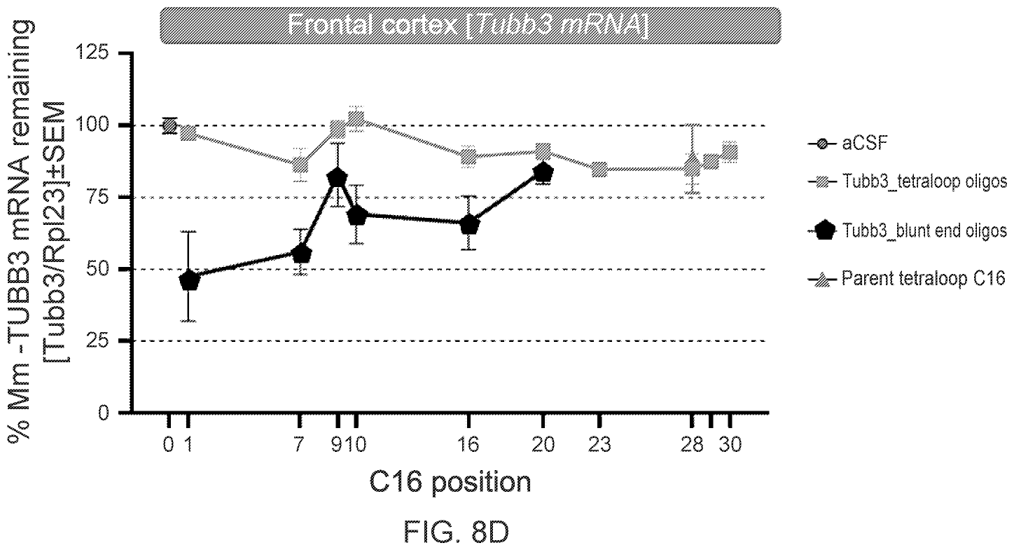

The mean in vivo reduction of Tubb3 mRNA observed in this example and Example 3 was compared (FIGS. 8A-8D). Blunt-end RNAi oligonucleotide-lipid conjugates generally had activity in CNS regions both proximal (e.g., lumbar spinal cord) and distal to the site of injection (e.g., medulla, hippocampus, frontal cortex) (FIGS. 8A-8D). Blunt-end oligonucleotides with a lipid conjugate at the 5′ terminal position of the sense strand (P1) or at the internal P7 position of the sense strand resulted in the highest levels of reduction of neuronal Tubb3 mRNA in the lumbar spinal cord (FIG. 8A), medulla (FIG. 8B), hippocampus (FIG. 8C), and frontal cortex (FIG. 8D). These results demonstrate the ability of blunt-end RNAi oligonucleotide-lipid conjugates having a lipid conjugated either at the 5′ terminal position of the sense strand (P1), at internal positions of the sense strand (e.g., P7, P9, P10, P16), or at the 3′ terminal position of the sense strand (P20), to reduce neuronal target gene (e.g., Tubb3) expression in the CNS.

Tetraloop RNAi oligonucleotide-lipid conjugates described in this example reduced neuronal Tubb3 mRNA expression in the lumbar spinal cord (FIG. 8A), a CNS region proximal to the site of injection. Tetraloop RNAi oligonucleotide-lipid conjugates generally reduced neuronal Tubb3 mRNA expression to a lesser extent than blunt-end RNAi oligonucleotide-lipid conjugates in the medulla, with the exception for position P20 conjugates (FIG. 8B). Tetraloop RNAi oligonucleotide-lipid conjugates reduced Tubb3 mRNA expression to a lesser extent than the blunt-end RNAi oligonucleotide-lipid conjugates in CNS regions distal to the site of injection (e.g., hippocampus, frontal cortex). Without wishing to be bound by theory, the observation that tetraloop RNAi oligonucleotides-lipid conjugates reduce neuronal target gene expression (e.g., Tubb3 expression) in a region of the CNS that is proximal to the site of administration (e.g., spinal cord) suggests such RNAi oligonucleotide-lipid conjugates may be useful to treat a specific disease or disorder (e.g., a spinal cord disease or disorder), wherein it is desirable, useful, or necessary to localize and/or restrict reduction of a corresponding disease or disorder-associated target gene or target mRNA to a particular region of the CNS (e.g., spinal cord or spinal cord structures such as dorsal root ganglion).

### Example 5: Positional Effects of Lipid Conjugation on the In Vitro Activity of Blunt-End and Tetraloop RNAi Oligonucleotide-Lipid Conjugates

The ability of RNAi oligonucleotide-lipid conjugates comprising a blunt-end or a tetraloop to reduce Tubb3 expression was evaluated in vitro. Specifically, Neuro2a cells were cultured with blunt-end or tetraloop RNAi oligonucleotides conjugated to a C16 lipid at positions P1, P7, P9, P10, P16, or P20, or with the reference tetraloop oligonucleotide conjugated at P28, as provided in Tables 1 and 2 at molar concentrations of 100 nM to 100 pM for 24 hours. Following treatment, Tubb3 mRNA was measured as described in Example 3.

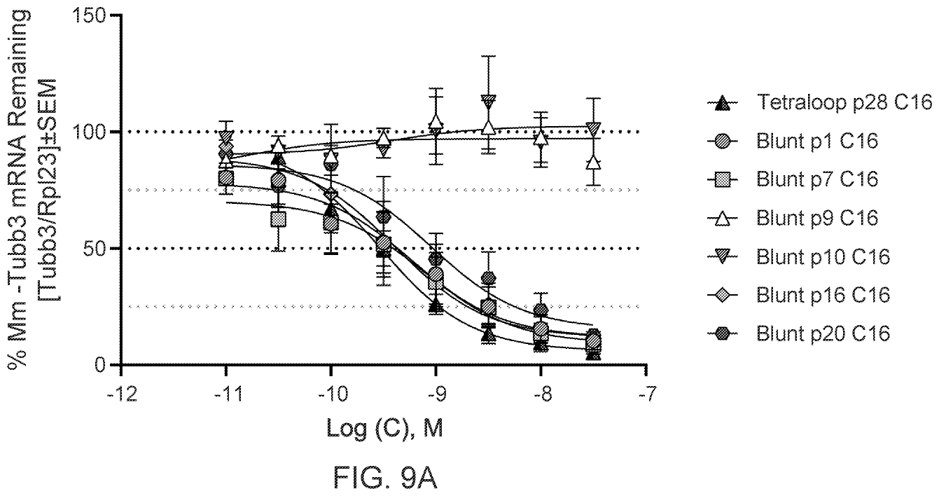

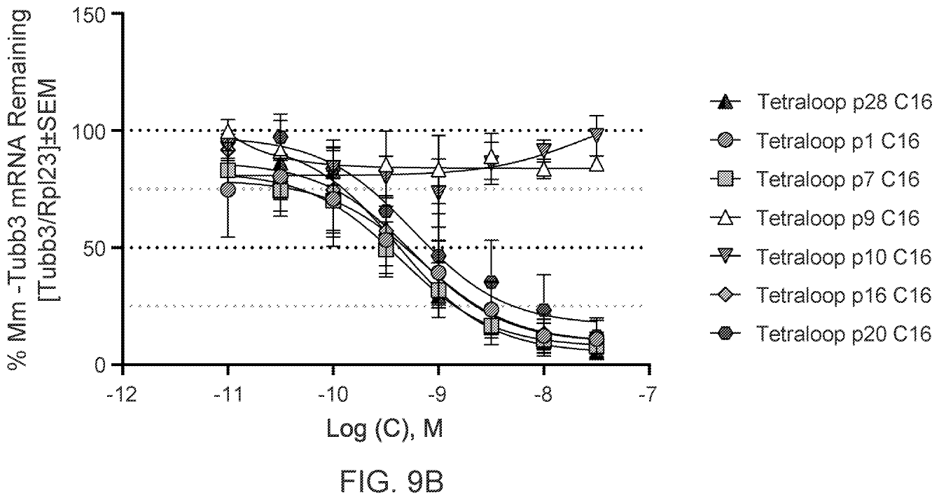

Treatment of cultured Neuro2a cells with blunt-end or tetraloop RNAi oligonucleotides conjugated to a C16 lipid at positions P1, P7, P16, or P20, or the reference tetraloop oligonucleotide conjugated at P28, reduced Tubb3 mRNA in a concentration-dependent matter (FIGS. 9A-9B). Treatment of cultured Neuro2a cells with either blunt-end or tetraloop RNAi oligonucleotides conjugated to a C16 lipid at positions P9 or P10 did not result in significant reduction of Tubb3 mRNA at any of the concentrations tested.

These results demonstrate that blunt-end and tetraloop RNAi oligonucleotides conjugated with a C16 lipid at positions P1, P7, P16, or P20, and the reference tetraloop oligonucleotide conjugated at P28, reduce Tubb3 mRNA in cultured Neuro2a cells to a similar extent when tested at equivalent molar concentrations. Further, these results suggest that lipid conjugation to either blunt-end or tetraloop RNAi oligonucleotides at positions P9 and P10 results in a RNAi oligonucleotide-lipid conjugate having a decreased ability to reduce target gene expression in a cell, as indicated by the lack of significant reduction of Tubb3 mRNA in Neuro2a cells when treated with these RNAi oligonucleotide-lipid conjugates. Taken together, the results shown in FIGS. 9A-9B demonstrate that the ability of blunt-end or tetraloop RNAi oligonucleotide-lipid conjugates having a lipid conjugated either at the 5′ terminal position of the sense strand (e.g., PT), at internal positions of the sense strand (e.g., P7, P16, P20), or at position P28 of the sense strand of a tetraloop RNAi oligonucleotide, to reduce target gene expression in a cell is about equivalent. Further, the results shown in FIGS. 9A-9B demonstrate that lipid conjugation of RNAi oligonucleotides at certain positions on the sense strand (e.g., P9, P10) disrupt their ability to reduce target gene expression (e.g., potentially due to an effect on their ability to function in the cellular RNAi pathway) in a cell.

TABLE 3
Lipid-conjugated RNAi Oligonucleotides

| | SEQ ID NO | | | |
|---|---|---|---|---|
| Position of | | Blunt-end | | Tetraloop |
| Lipid- | Blunt-end | antisense | Tetraloop | Antisense |
| Conjugation | Sense Strand | Strand | Sense Strand | Strand |
| P1 | 4 | 10 | 11 | 10 |
| P7 | 5 | 10 | 12 | 10 |
| P16 | 8 | 10 | 15 | 10 |
| P20 | 9 | 10 | 16 | 10 |

### Sequence Listing

| | | | SEQ ID |
|---|---|---|---|
| Name | Description | Sequence | NO |
| TUBB3 | Sense Strand | AGUGUGAGAAUUGUGACUGA | 1 |
| TUBB3 | Antisense Strand | UCAGUCACAAUUCUCACACUGG | 2 |
| P28 C16 | C16 Position 1 | [mAs][mG][mU][mG][mU][mG][mA][fG][fA][fA] | 3 |
| | modified Sense | [fU][mU][mG][mU][mG][mA][mC][mU][mG][mA] | |
| | strand | [mG][mC][mA][mG][mC][mC][mG][ademA-C16] | |
| | | [mA\|[mA][mG][mG\|[mC\|[mU][mG][mC] | |
| P1 C16 | C16 Position 1 | [ademAs-C16][mG][mU][mG][mU][mG] | 4 |
| | modified Sense | [mA][fG][fA][fA][fU][mU][mG][mU][mG][mA] | |
| | strand | [mC][mUs][mGs][mA] | |
| P7 C16 | C16 Position 7 | [mAs][mG][mU][mG][mU][mG][ademA-C16] | 5 |
| | modified Sense | [fG][fA][fA][fU][mU][mG][mU][mG][mA][mC] | |
| | strand | [mUs][mGs][mA] | |
| P9 C16 | C16 Position 9 | [mAs][mG][mU][mG][mU][mG][mA][fG] | 6 |
| | modified Sense | [ademA-C16][fA][fU][mU][mG] | |
| | strand | [mU][mG][mA][mC][mUs][mGs][mA] | |
| P10 C16 | C16 Position 10 | [mAs][mG][mU][mG][mU][mG] | 7 |
| | modified Sense | [mA][fG][fA][ademA-C16][fU][mU] | |
| | strand | [mG][mU][mG][mA][mC][mUs][mGs][mA] | |
| P16 C16 | C16 Position 16 | [mAs][mG][mU][mG][mU][mG][mA][fG][fA][fA] | 8 |
| | modified Sense | [fU][mU][mG][mU][mG][ademA-C16][mC] | |
| | strand | [mUs][mGs][mA] | |
| P20 C16 | C16 Position 20 | [mAs][mG][mU][mG][mU][mG][mA][fG] | 9 |
| | modified Sense | [fA][fA][fU][mU][mG][mU][mG][mA][mC][mUs] | |
| | strand | [mGs][ademAs-C16] | |
| C16 | Antisense Strand | [MePhosphonate-4O-mUs][fCs][fAs] | 10 |
| antisense | | [fG][fU][mC][fA][mC][mA][fA][mU][mU][mC] | |
| strand | | [fU][mC][mA][mC][mA][mC][mUs][mGs][mG] | |
| P1 C16 | C16 Position 1 | [ademAs-C16][mG][mU][mG][mU][mG][mA] | 11 |
| Stem | Modified Sense | [fG][fA][fA][fU][mU][mG][mU][mG][mA][mC] | |
| Loop | Strand | [mU][mG][mA][mG][mC][mA][mG][mC][mC][mG] | |
| | | [mA\|[mA][mA][mG][mG\|[mC\|[mU][mG][mC] | |
| P7 C16 | C16 Position 1 | [mAs][mG][mU][mG][mU][mG][ademA-C16] | 12 |
| Stem | Modified Sense | [fG][fA][fA][fU][mU][mG][mU][mG][mA][mC] | |
| Loop | Strand | [mU][mG][mA][mG][mC][mA][mG][mC][mC][mG] | |
| | | [mA][mA][mA][mG][mG][mC][mU][mG][mC] | |
| P9 C16 | C16 Position 9 | [mAs][mG][mU][mG][mU][mG][mA][fG][ademA- | 13 |
| Stem | Modified Sense | C16][fA][fU][mU][mG][mU][mG][mA][mC][mU] | |
| Loop | Strand | [mG][mA][mG][mC][mA][mG][mC][mC][mG][mA] | |
| | | [mA][mA][mG][mG][mC][mU][mG][mC] | |
| P10 C16 | C16 Position 10 | [mAs][mG][mU][mG][mU][mG][mA][fG][fA] | 14 |
| Stem | Modified Sense | [ademA-C16][fU][mU][mG][mU][mG][mA][mC] | |
| Loop | Strand | [mU][mG][mA][mG][mC][mA][mG][mC][mC] | |
| | | [mG][mA][mA][mA][mG][mG][mC][mU][mG][mC] | |
| P16 C16 | C16 Position 16 | [mAs][mG][mU][mG][mU][mG][mA][fG][fA][fA] | 15 |
| Stem | Modified Sense | [fU][mU][mG][mU][mG][ademA-C16][mC][mU] | |
| Loop | Strand | [mG][mA][mG][mC][mA][mG][mC][mC][mG][mA] | |
| | | [mA][mA][mG][mG][mC][mU][mG][mC] | |
| P20 C16 | C16 Position 20 | [mAs][mG][mU][mG][mU][mG][mA][fG][fA][fA] | 16 |
| Stem | Modified Sense | [fU][mU][mG][mU][mG][mA][mC][mU][mG] | |
| Loop | Strand | [ademA-C16][mG][mC][mA][mG][mC][mC][mG] | |
| | | [mA][mA][mA][mG][mG][mC][mU][mG][mC] | |
| P23 C16 | C16 Position 23 | [mAs][mG][mU][mG][mU][mG][mA][fG][fA][A] | 17 |
| Stem | Modified Sense | [fU][mU][mG][mU][mG][mA][mC][mU][mG][mA] | |
| Loop | Strand | [mG][mC][ademA-C16][mG][mC][mC][mG][mA] | |
| | | [mA][mA][mG][mG][mC][mU][mG][mC] | |
| P29 C16 | C16 Position 29 | [mAs][mG][mU][mG][mU][mG][mA][fG][fA][fA] | 18 |
| Stem | Modified Sense | [fU][mU][mG][mU][mG][mA][mC][mU][mG][mA] | |
| Loop | Strand | [mG][mC][mA][mG][mC][mC][mG][mA][ademA- | |
| | | C16][mA][mG][mG][mC][mU][mG][mC] | |
| P30 C16 | C16 Position 30 | [mAs][mG][mU][mG][mU][mG][mA][fG][fA][fA] | 19 |
| Stem | Modified Sense | [fU][mU][mG][mU][mG][mA][mC][mU][mG][mA] | |
| Loop | Strand | [mG][mC][mA][mG][mC][mC][mG][mA][mA] | |
| | | [ademA-C16][mG][mG][mC][mU][mG][mC] | |
| 36 mer | Unmodified | AGUGUGAGAAUUGUGACUGAGCAGCCGAA | 20 |
| | Sense Strand | AGGCUGC | |
| Stem loop | | GCAGCCGAAAGGCUGC | 21 |

1.-95. (canceled)

96. A double-stranded oligonucleotide comprising an antisense strand of 15-30 nucleotides in length and a sense strand of 15-50 nucleotides in length, wherein the antisense and sense strands form a duplex region of 15-30 base pairs, wherein the antisense strand comprises a region of complementarity to a neuronal mRNA target sequence, and wherein the sense strand comprises at least one lipid moiety, wherein the lipid moiety is a C16 hydrocarbon chain conjugated to the 2′ carbon of the ribose ring of the 5′ terminal nucleotide of the sense strand.

97. The double-stranded oligonucleotide of claim 96, wherein the C16 hydrocarbon chain is represented by

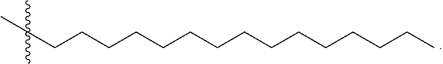 [SMILES: *CCCCCCCCCCCCCCC]

98. The double-stranded oligonucleotide of claim 96, wherein the double-stranded oligonucleotide comprises a blunt end, wherein the blunt end comprises the 3′ end of the sense strand and 5′ end of the antisense strand.

99. The double-stranded oligonucleotide of claim 96, wherein the sense strand is 20-22 nucleotides and the antisense strand is 22-24 nucleotides, and wherein the duplex region is 20-22 base pairs.

100. The double-stranded oligonucleotide of claim 96, wherein the double-stranded oligonucleotide further comprises a stem-loop, wherein the stem-loop comprises a nucleotide sequence represented by the formula: 5′-S1-L-S2-3′, wherein S1 is complementary to S2, and wherein L forms a loop between S1 and S2.

101. The double-stranded oligonucleotide of claim 100, wherein the sense strand is 36-38 nucleotides and the antisense strand is 22-24 nucleotides, and wherein the duplex region is 20 base pairs.

102. The double-stranded oligonucleotide of claim 100, wherein the sense strand is 36 nucleotides and comprises positions 1-36 from 5′ to 3′, and wherein the lipid moiety is conjugated at position 1 and wherein the duplex region is 20 base pairs.

103. The double-stranded oligonucleotide of claim 96, wherein each of the nucleotides of the sense strand and the antisense strand comprise a 2′-modification except the 5′-terminal nucleotide of the sense strand, and wherein the 2′-modification is selected from 2′-aminoethyl, 2′-fluoro, 2′-O-methyl, 2′-O-methoxyethyl, and 2′-deoxy-2′-fluoro-β-d-arabinonucleic acid.

104. The double-stranded oligonucleotide of claim 96, wherein the sense strand comprises 20 nucleotides with positions 1-20 from 5′ to 3′, wherein each of positions 8-11 comprise a 2′-fluoro modification.

105. The double-stranded oligonucleotide of claim 96, wherein the sense strand comprises 36 nucleotides with positions 1-36 from 5′ to 3′, wherein each of positions 8-11 comprise a 2′-fluoro modification.

106. The double-stranded oligonucleotide of claim 96, wherein the antisense strand comprises 22 nucleotides with positions 1-22 from 5′ to 3′, and wherein each of positions 2, 3, 4, 5, 7, 10 and 14 comprise a 2′-fluoro modification.

107. The double-stranded oligonucleotide of claim 96, wherein the double-stranded oligonucleotide comprises at least one phosphorothioate linkage.

108. The double-stranded oligonucleotide of claim 107, wherein the antisense strand comprises a phosphorothioate linkage:
(i) between positions 1 and 2, and between positions 2 and 3;
(ii) between positions 1 and 2, between positions 2 and 3, and between positions 3 and 4;
(iii) between positions 1 and 2; and/or
(iv) between positions 20 and 21 and between positions 21 and 22;
wherein positions are numbered 1-22 from 5′ to 3′.

109. The double-stranded oligonucleotide of claim 96 wherein the sense strand is 20 nucleotides in length, and wherein the sense strand comprises a phosphorothioate linkage between positions 18 and 19, and between positions 19 and 20, wherein positions are numbered 1-20 from 5′ to 3′.

110. The double-stranded oligonucleotide of claim 96, wherein the antisense strand comprises a phosphorylated nucleotide at the 5′ terminus, wherein the phosphorylated nucleotide comprises uridine or adenosine.

111. The double-stranded oligonucleotide of claim 96, wherein the 4′-carbon of the sugar of the 5′-nucleotide of the antisense strand comprises a phosphate analog.

112. The double-stranded oligonucleotide of claim 96, wherein the double-stranded oligonucleotide reduces expression of a target mRNA in a neuron or population of neurons in the spinal cord relative to expression of the target mRNA in other regions of the central nervous system (CNS).

113. A pharmaceutical composition comprising the double-stranded oligonucleotide of claim 96, and a pharmaceutically acceptable carrier, delivery agent or excipient.

114. A method for treating a subject having a disease, disorder, or condition associate with expression of a neuronal mRNA, the method comprising administering intrathecally to the subject a therapeutically effective amount of the double-stranded oligonucleotide of claim 96, thereby treating the subject.

115. A method for reducing expression of a neuronal mRNA in a cell, a population of cells or a subject, the method comprising the step of:
i. contacting the cell or the population of cells with the double-stranded oligonucleotide of claim 96; or
ii. administering to the subject the double-stranded oligonucleotide of claim 96, wherein administering is intrathecal;
and wherein the method results in a reduction in expression of the neuronal mRNA comprising reducing an amount or level of mRNA, an amount or level of protein, or both.

116. A double-stranded oligonucleotide comprising an antisense strand of 22-24 nucleotides in length and a sense strand of 20-22 nucleotides in length, wherein the antisense and sense strands form an asymmetric duplex region of 20-22 base pairs, wherein the double-stranded oligonucleotide comprises a blunt-end comprising the 3′ end of the sense strand and 5′ end of the antisense strand, wherein the antisense strand comprises a region of complementarity to at least 15 consecutive nucleotides of a neuronal mRNA target sequence, wherein the sense strand comprises at least one lipid moiety conjugated to the 2′ carbon of the ribose ring of the 5′ terminal nucleotide the sense strand, wherein the lipid moiety is a C16 hydrocarbon chain represented by, 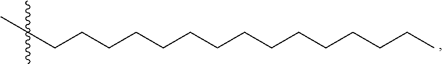 [SMILES: *CCCCCCCCCCCCCCC]
and wherein the antisense strand comprises a 2-nucleotide overhang on the 3′ end.

117. The double-stranded oligonucleotide of claim 116, wherein each of the nucleotides of the sense strand and the antisense strand comprise a 2′-modification except the 5′-terminal nucleotide of the sense strand, and wherein the 2′-modification is selected from 2′-aminoethyl, 2′-fluoro, 2′-O-methyl, 2′-O-methoxyethyl, and 2′-deoxy-2′-fluoro-β-d-arabinonucleic acid.

118. The double-stranded oligonucleotide of claim 116, wherein the sense strand comprises 20 nucleotides with positions 1-20 from 5′ to 3′, wherein each of positions 8-11 comprise a 2′-fluoro modification.

119. The double-stranded oligonucleotide of claim 116, wherein the antisense strand comprises 22 nucleotides with positions 1-22 from 5′ to 3′, and wherein each of positions 2, 3, 4, 5, 7, 10 and 14 comprise a 2′-fluoro modification.

120. The double-stranded oligonucleotide of claim 116, wherein the double-stranded oligonucleotide comprises at least one phosphorothioate linkage.

121. The double-stranded oligonucleotide of claim 120, wherein the antisense strand comprises a phosphorothioate linkage:
(i) between positions 1 and 2, and between positions 2 and 3;
(ii) between positions 1 and 2, between positions 2 and 3, and between positions 3 and 4;
(iii) between positions 1 and 2; and/or
(iv) between positions 20 and 21 and between positions 21 and 22;
wherein positions are numbered 1-22 from 5′ to 3′.

122. The double-stranded oligonucleotide of claim 116 wherein the sense strand is 20 nucleotides in length, and wherein the sense strand comprises a phosphorothioate linkage between positions 18 and 19, and between positions 19 and 20, wherein positions are numbered 1-20 from 5′ to 3′.

123. The double-stranded oligonucleotide of claim 116, wherein the antisense strand comprises a phosphorylated nucleotide at the 5′ terminus, wherein the phosphorylated nucleotide comprises uridine or adenosine.

124. The double-stranded oligonucleotide of claim 116, wherein the 4′-carbon of the sugar of the 5′-nucleotide of the antisense strand comprises a phosphate analog.

125. The double-stranded oligonucleotide of claim 116, wherein the double-stranded oligonucleotide reduces expression of a target mRNA in a neuron or population of neurons in the spinal cord relative to expression of the target mRNA in other regions of the central nervous system (CNS).

126. A double-stranded oligonucleotide comprising an antisense strand of 15-30 nucleotides in length and a sense strand of 15-50 nucleotides in length, wherein the antisense and sense strands form a duplex region of 15-30 base pairs, wherein the antisense strand comprises a region of complementarity to a neuronal mRNA target sequence, wherein the sense strand comprises at least one lipid moiety, and wherein the lipid moiety is a hydrocarbon chain.


SEQUENCE LISTING (21 sequences)

SEQ ID NO: 1 - RNA, 20 nt, Artificial Sequence
  Tubulin Beta 3 Class III (Tubb3) sense strand
agugugagaauugugacuga

SEQ ID NO: 2 - RNA, 22 nt, Artificial Sequence
  Tubulin Beta 3 Class III (Tubb3) antisense strand
ucagucacaauucucacacugg

SEQ ID NO: 3 - RNA, 36 nt, Artificial Sequence
  C16 Position 1 modified Sense strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): C16 lipid conjugate attached
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified
agugugagaauugugacugagcagccgaaaggcugc

SEQ ID NO: 4 - RNA, 20 nt, Artificial Sequence
  C16 Position 1 modified Sense strand
  misc_feature (1)..(2): linked via phosphorothioate
  modified_base (1)..(1): C16 lipid conjugate attached
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
agugugagaauugugacuga

SEQ ID NO: 5 - RNA, 20 nt, Artificial Sequence
  C16 Position 7 modified Sense strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): C16 lipid conjugate attached
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  misc_feature (18)..(20): linked via phosphorothioate
  modified_base (18)..(18): 2′-O-methyl modified
  modified_base (19)..(19): 2′-O-methyl modified
  modified_base (20)..(20): 2′-O-methyl modified
agugugagaauugugacuga

SEQ ID NO: 6 - RNA, 20 nt, Artificial Sequence
  C16 Position 9 modified Sense strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): C16 lipid conjugate attached
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  misc_feature (18)..(20): linked via phosphorothioate
  modified_base (18)..(18): 2′-O-methyl modified
  modified_base (19)..(19): 2′-O-methyl modified
  modified_base (20)..(20): 2′-O-methyl modified
agugugagaauugugacuga

SEQ ID NO: 7 - RNA, 20 nt, Artificial Sequence
  C16 Position 10 modified Sense strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): C16 lipid conjugate attached
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  misc_feature (18)..(20): linked via phosphorothioate
  modified_base (18)..(18): 2′-O-methyl modified
  modified_base (19)..(19): 2′-O-methyl modified
  modified_base (20)..(20): 2′-O-methyl modified
agugugagaauugugacuga

SEQ ID NO: 8 - RNA, 20 nt, Artificial Sequence
  C16 Position 16 modified Sense strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): C16 lipid conjugate attached
  modified_base (17)..(17): 2′-O-methyl modified
  misc_feature (18)..(20): linked via phosphorothioate
  modified_base (18)..(18): 2′-O-methyl modified
  modified_base (19)..(19): 2′-O-methyl modified
  modified_base (20)..(20): 2′-O-methyl modified
agugugagaauugugacuga

SEQ ID NO: 9 - RNA, 20 nt, Artificial Sequence
  C16 Position 20 modified Sense strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  misc_feature (18)..(20): linked via phosphorothioate
  modified_base (18)..(18): 2′-O-methyl modified
  modified_base (19)..(19): 2′-O-methyl modified
  modified_base (20)..(20): C16 lipid conjugate attached
agugugagaauugugacuga

SEQ ID NO: 10 - RNA, 22 nt, Artificial Sequence
  Antisense Strand
  misc_feature (1)..(4): linked via phosphorothioate
  modified_base (1)..(1): 4′-O-monomethylphosphonate-2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
ucagucacaauucucacacugg

SEQ ID NO: 11 - RNA, 36 nt, Artificial Sequence; identical to SEQ ID NO: 3
  C16 Position 1 Modified Sense Strand
  misc_feature (1)..(2): linked via phosphorothioate
  modified_base (1)..(1): C16 lipid conjugate attached
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 12 - RNA, 36 nt, Artificial Sequence; identical to SEQ ID NO: 3
  C16 Position 1 Modified Sense Strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): C16 lipid conjugate attached
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 13 - RNA, 36 nt, Artificial Sequence; identical to SEQ ID NO: 3
  C16 Position 9 Modified Sense Strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): C16 lipid conjugate attached
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 14 - RNA, 36 nt, Artificial Sequence; identical to SEQ ID NO: 3
  C16 Position 10 Modified Sense Strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): C16 lipid conjugate attached
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 15 - RNA, 36 nt, Artificial Sequence; identical to SEQ ID NO: 3
  C16 Position 16 Modified Sense Strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): C16 lipid conjugate attached
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 16 - RNA, 36 nt, Artificial Sequence; identical to SEQ ID NO: 3
  C16 Position 20 Modified Sense Strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): C16 lipid conjugate attached
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 17 - RNA, 36 nt, Artificial Sequence; identical to SEQ ID NO: 3
  C16 Position 23 Modified Sense Strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): C16 lipid conjugate attached
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 18 - RNA, 36 nt, Artificial Sequence; identical to SEQ ID NO: 3
  C16 Position 29 Modified Sense Strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): C16 lipid conjugate attached
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 19 - RNA, 36 nt, Artificial Sequence; identical to SEQ ID NO: 3
  C16 Position 30 Modified Sense Strand
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): C16 lipid conjugate attached
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 20 - RNA, 36 nt, Artificial Sequence; identical to SEQ ID NO: 3
  Unmodified Sense Strand

SEQ ID NO: 21 - RNA, 16 nt, Artificial Sequence
  stemloop
gcagccgaaaggcugc